(19) United States
(12) Patent Application Publication
(10) Pub. No.: US 2025/0011879 A1
(43) Pub. Date: Jan. 9, 2025

(54) METHODS AND COMPOSITIONS RELATED TO INFLAMMATORY GENE PANEL

(71) Applicant: Ohio State Innovation Foundation, Columbus, OH (US)

(72) Inventors: Jose OTERO, Dublin, OH (US); Wesley WANG, Columbus, OH (US); Diana THOMAS, Dublin, OH (US); Raghu MACHIRAJU, Dublin, OH (US)

(21) Appl. No.: 18/712,422

(22) PCT Filed: Dec. 2, 2022
(86) PCT No.: PCT/US22/51671

§ 371 Date: May 22, 2024

Related U.S. Application Data
(60) Provisional application No. 63/285,339, filed on Dec. 2, 2021.

(51) Int. Cl. C12Q 1/6886

(52) U.S. Cl. CPC C12Q 1/6886; C12Q 2600/112; C12Q 2600/158; G01N 2800/52

(57) ABSTRACT

Glioblastomas (GBMs) are primary brain lesions with poor overall survival rates. Nearly 95% of patients die within 5 years of primary diagnosis. One of the leading factors to this high mortality is the inevitable nature of cancer recurrence. Disclosed herein are methods and compositions related to the identification and treatment of glioblastoma. Specifically, disclosed herein are methods for identifying progressing glioblastoma, and distinguishing it from pseudo-progression.

### CROSS-REFERENCE TO RELATED APPLICATIONS

This application claims the benefit of priority to U.S. Provisional Application No. 63/285,339, filed Dec. 2, 2021, which is hereby incorporated by reference herein in its entirety.

### BACKGROUND

Glioblastomas (GBMs) are primary brain lesions with poor overall survival rates. Nearly 95% of patients die within 5 years of primary diagnosis. One of the leading factors to this high mortality is the inevitable nature of cancer recurrence. In consequence, early detection of cancer return is critical to patient survival and monitored using serial magnetic resonance imaging (MRI) to detect areas of contrast enhancement indicatory of possible cancer return. However, similarly presenting pseudo-progression (psPD) complicates monitoring as such contrast enhancing lesions present identically under MRI. This paradigm complicates patient management clinically as psPD may be conservatively managed with monitoring and/or steroids while true cancer progression (PD) must be aggressively managed with surgery and treatment revision. In turn, histologic confirmation by neuropathology is critical in resolving novel MRI enhancements, but equally challenged in accurately classifying response as practical clinical guidelines do not exist. What is needed in the art are methods and compositions to better distinguish true cancer progression from pseudo-progression.

### FIELD

The present disclosure relates inflammatory gene panels and uses thereof.

### SUMMARY

The present disclosure provides an inflammatory gene panel and methods for uses and manufacture thereof.

In one aspect, disclosed herein is a method for identifying a subject with progressing glioblastoma versus pseudo-progressing glioblastoma, the method comprising a) determining an expression status of one or more genes selected from 1000 genes or more isolated from a biological sample from the subject, b) using the expression status to determine an expression score relative to a normal subject, c) identifying a subject with an expression score greater than 25% as being at risk of having progressing glioblastoma relative to a normal subject, d) identifying a subject with an expression score less than 25% as being at risk of having pseudo-progressing glioblastoma relative to a normal subject, and e) treating the subject based on the expression score.

In one embodiment, the expression score is determined from the expression status of a subset of genes. In other embodiments, the expression score is determined from the expression status of a subset of 10 genes. In other embodiments, the expression score is determined from the expression status of a subset of 100 genes. In other embodiments, the expression score is determined from the expression status of a subset of 500 genes. In other embodiments, the expression score is determined from the expression status of a subset of 1000 genes.

In another embodiment, the subject identified with progressing glioblastoma is treated with surgery, chemotherapy, radiation, immunotherapy, gene therapy, or a combination thereof. In another embodiment, the subject identified with pseudo-progressing glioblastoma is treated with monitoring and/or steroids.

In another embodiment, the biological sample is selected from the group consisting of blood, plasma, serum, cerebrospinal fluid, urine, saliva, stool, synovial fluid, a solid tissue sample, and a cell culture sample.

In one aspect, disclosed herein is a method for identifying a subject with progressing glioblastoma and further diagnosing them to confirm that the subject has progressing glioblastoma, the method comprising a) determining an expression status of one or more genes selected from 1000 genes or more isolated from a biological sample from the subject, b) using the expression status to determine an expression score relative to a normal subject, c) identifying a subject with an expression score greater than 25% as being at risk of having progressing glioblastoma relative to a normal subject, and subjecting the subject to a further diagnosis.

In one embodiment, the expression score is determined from the expression status of a subset of genes. In another embodiment, the expression score is determined from the expression status of a subset of 10 genes. In another embodiment, the expression score is determined from the expression status of a subset of 100 genes. In another embodiment, the expression score is determined from the expression status of a subset of 500 genes. In another embodiment, the expression score is determined from the expression status of a subset of 1000 genes.

In another embodiment, said further diagnosis comprises an MRI. In other embodiments, after the further diagnosis, the subject is treated with surgery, chemotherapy, radiation, immunotherapy, gene therapy, or a combination thereof. In some embodiments, the biological sample is selected from the group consisting of blood, plasma, serum, cerebrospinal fluid, urine, saliva, stool, synovial fluid, a solid tissue sample, and a cell culture sample.

In one aspect, disclosed herein is a method for monitoring progression in a subject undergoing a treatment for a glioblastoma and adjusting said treatment, the method comprising a) determining an expression status of one or more genes selected from 1000 genes or more isolated from a biological sample from the subject, b) identifying a subject with an expression score greater than 25% as being at risk of having progressing glioblastoma relative to a normal subject, c) giving the subject a first treatment based on the expression score, d) repeating steps a)-d) to monitor a change in the expression score, and e) giving the subject a second treatment if the expression score remains or increases above 25%.

In one embodiment, the expression score is determined from the expression status of a subset of genes. In another embodiment, the expression score is determined from the expression status of a subset of 10 genes. In another embodiment, the expression score is determined from the expression status of a subset of 100 genes. In another embodiment, the expression score is determined from the expression status of a subset of 500 genes. In another embodiment, the expression score is determined from the expression status of a subset of 1000 genes.

In some embodiments, the second treatment is given in combination with the first treatment. In another embodiment, the second treatment is given alone. In another embodiment, the first treatment and the second treatment is surgery, chemotherapy, radiation, immunotherapy, gene therapy, or a combination thereof. In another embodiment, the biological sample is selected from the group consisting of blood, plasma, serum, cerebrospinal fluid, urine, saliva, stool, synovial fluid, a solid tissue sample, and a cell culture sample. In another embodiment, the subject is a human.

In another aspect, disclosed herein is a diagnostic kit or assay for identifying a subject with progressing glioblastoma, wherein the kit or assay comprises components comprising one or more detection means for detecting one or more biomarkers selected from the group comprising CENPF, BIRC5, GAS1, ANLN, RRM2, MKI67, SOX11, CCNB1, UBE2C, CQL11A, MELK, AQP9, NGFR, TREM1, AREG, MARCO, IL1R2, CCL20, NOTCH1, CDK6, ENO1, TREM, PDGFA, HELLS, FANCA, DNMT1, IGF2R, BNIP3L, CLEC5A, CDC25C, PIK3R2, SPP1, BCL2L1, CDH11, FBP1, BIRC5, POLD1, IL1R2, BRIP1, P4HA1, ZEB1, TCF3, EZH2, S100A9, or combinations thereof.

In one embodiment, said detection means comprise enzyme linked immunosorbent assay (ELISA), immunofluorescence, western blotting, immunodiffusion, polymerase chain reaction (PCR), or flow cytometry. In other embodiments, said detection means further comprises one or more reagents for determining the presence of one or more biomarkers selected from 1000 or more genes, said detection means further comprises one or more reagents for determining the presence of one or more biomarkers selected from 1000 or more genes.

In another embodiment, the one or more biomarkers is selected from a subset of genes. In another embodiment, the one or more biomarkers is selected from a subset of 10 genes. In another embodiment, the one or more biomarkers is selected from a subset of 100 genes. In another embodiment, the one or more biomarkers is selected from a subset of 500 genes. In another embodiment, the one or more biomarkers is selected from a subset of 1000 genes.

In some embodiments, said reagent is a monoclonal antibody or any antigen binding fragment thereof coupled to a detectable reagent. In some embodiments, the detectable reagent comprises an enzyme, a fluorescent label, a radiolabel, or a chemiluminescent label. In other embodiments, said biomarkers are detected in a biological sample from the subject.

In another embodiment, the biological sample is selected from the group consisting of blood, plasma, serum, cerebrospinal fluid, urine, saliva, stool, synovial fluid, a solid tissue sample, and a cell culture sample. In another embodiment, the subject is a human.

### BRIEF DESCRIPTION OF FIGURES

The accompanying figures, which are incorporated in and constitute a part of this specification, illustrate several aspects described below.

FIGS. 1A-1C show the clustering analysis of recurrent GBMs indicates differential survival based upon stratification of immune evasion, stem cell-like and proliferative activity of cells found in tumor. FIG. 1A shows the kMeans unsupervised clustering of GBM tumors based of RNAseq signatures of aforementioned genes families. Cluster count was based upon gap-statistic calculation, where individual points represent single tumor cases. FIG. 1B shows the survival curves of patient clusters. FIG. 1C shows the top 15 gene signatures used to drive classification of clusters using random forest based modeling.

FIGS. 2A-2C show the differential expression and pathway analysis of progressive and pseudoprogressive GBM lesions indicate differential immune and stem-cell activity. FIG. 2A shows the volcano plot analysis shows differentially expressed genes with genes on the right being upregulated and genes on the left being downregulated. Horizontal dotted lines represent p=0.05 with genes above representing p>0.05. Vertical lines represent log 2 fold changes of −1.5 and 1.5. FIG. 2B shows the pathway analysis represents top changed pathways with dots on left representing upregulated pathways and dots on right representing downregulated pathways Color gradient reflects p-value of pathway expression difference between PD and psPD and dot size represents the number of genes represented by a pathway. FIG. 2C shows the network analysis displays pathways upregulated in red and downregulated in blue. Colored nodes represent genes with respective EntrezIDs with common nodes representing linked functions. Gene nodes with multiple lines represent overlapping functions.

FIG. 3 shows immune and stem cell related gene signatures accurately stratify disease response. Boruta analysis was utilized to identify redundant and non-important gene signatures. Remaining gene signatures were subsequently utilized to predict disease using a random forest learning algorithm and assessed with confusion matrix scores and AUC.

FIGS. 4A-4C show the post-surgical cancer lesions sort into novel clusters based on expression profiles independent of ground truth labels. FIG. 4A shows the tSNE reduced data was clustered utilizing kMeans. FIG. 4B shows the optimal cluster number was identified using a silhouette method with the dotted line representing optimal clusters. FIG. 4C shows the tSNE plot was relabeled with sample data labels with PD representing progression and psPD representing pseudoprogression.

FIGS. 5A-5C show the expression and pathway analysis of Cluster 1 and Cluster 2 indicate prominent differential immune response activity. FIG. 5A shows the volcano plot analysis shows differentially expressed genes with genes on the right being upregulated and genes on the left being downregulated. Horizontal dotted lines represent p=0.05 with genes above representing p>0.05. Vertical lines represent log 2 fold changes of −1.5 and 1.5. FIG. 5B shows the pathway analysis represents top changed pathways with dots on left representing upregulated pathways and dots on right representing downregulated pathways. Color gradient reflects p-value of pathway expression difference between PD and psPD and dot size represents the number of genes represented by a pathway. FIG. 5C shows the network analysis displays pathways upregulated in red and downregulated in blue Colored nodes represent genes with respective EntrezIDs with common nodes representing linked functions. Gene nodes with multiple lines represent overlapping functions. In consequence, differential expression, pathway, and network analyses were performed comparing cluster 1 to cluster 2. Notably, immune cell response pathways were increased in expression in cluster 1 compared to cluster 2, highlighting a use of immune cell response to differentiate the 2 pathways. In assaying the predictive capability of a random forest algorithm in predicting a cluster, confusion matrix accuracy scores were 100% with an AUC of 1. The decision modeling was heavily driven by expression of genes related to immune response.

FIG. 6 shows the recurrent CGGA GBM dendrogram and heat map.

FIG. 7 shows the module-trait relationships.

FIGS. 8A-8D show the module membership versus gene significance for different modules. (FIG. 8A is light steel blue, FIG. 8B is medium purple, FIG. 8C is brown, and FIG. 8D is orange).

FIG. 9 shows the correlation from FIG. 8A-8D and shows the modules most correlated with the cluster variation are related to immune function.

FIG. 10 shows the immune response gene signatures accurately stratify clusters. Boruta analysis was utilized to identify redundant and non-important gene signatures. Remaining gene signatures were subsequently utilized to predict disease using a random forest learning algorithm and assessed with confusion matrix scores and AUC.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising,” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a,” “an,” “the,” include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipien.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “treat,” “treating,” “treatment,” or any variations thereof refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Comprising” is intended to mean that the compositions, methods, etc. include the recited elements, but do not exclude others “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purpose. A control can be “positive” or “negative.” For example, where the purpose of the experiment is to determine a correlation of an altered expression level of a proto-oncogene with a particular type of cancer, it is generally preferable to use a positive control (a subject or a sample from a subject, carrying such alteration and exhibiting syndromes characteristic of that disease), and a negative control (a subject or a sample from a subject lacking the altered expression and clinical syndrome of that disease).

“Expression” as used herein refers to the process by which information from a gene is used in the synthesis of a functional gene product that enables it to produce a peptide/protein end product, and ultimately affect a phenotype, as the final effect.

The term “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The term “detect,” “detection,” or “detecting” refers to an output signal released for the purpose of sensing of physical phenomenon. An event or change in environment is sensed and signal output released in the form of light.

The term “antibody” is used in the broadest sense, and specifically covers monoclonal antibodies (including full length monoclonal antibodies), polyclonal antibodies, and multispecific antibodies (e.g., bispecific antibodies). Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity Native antibodies and immunoglobulins are usually heterotetrameric glycoproteins of about 150,000 daltons, composed of two identical light (L) chains and two identical heavy (H) chains. Each heavy chain has at one end a variable domain (V_{H}) followed by a number of constant domains. Each light chain has a variable domain at one end (V_{L}) and a constant domain at its other end.

The term “antibody fragment” or “antigen binding fragment” refers to a portion of a full-length antibody or antigen, generally the target binding or variable region. Examples of antibody fragments include Fab, Fab′, F(ab′)₂ and Fv fragments. The phrase “functional fragment or analog” of an antibody is a compound having qualitative biological activity in common with a full-length antibody. For example, a functional fragment or analog of an anti-IgE antibody is one which can bind to an IgE immunoglobulin in such a manner so as to prevent or substantially reduce the ability of such molecule from having the ability to bind to the high affinity receptor, FcεRI. As used herein, “functional fragment” with respect to antibodies, refers to Fv, F(ab) and F(ab′)₂ fragments. An “Fv” fragment is the minimum antibody fragment which contains a complete target recognition and binding site. This region consists of a dimer of one heavy and one light chain variable domain in a tight, non-covalent association (V_{H}-V_{L} dimer). It is in this configuration that the three CDRs of each variable domain interact to define a target binding site on the surface of the V_{H}-V_{L} dimer. Collectively, the six CDRs confer target binding specificity to the antibody. However, even a single variable domain (or half of an Fv comprising only three CDRs specific for a target) has the ability to recognize and bind target, although at a lower affinity than the entire binding site. “Single-chain Fv” or “sFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains which enables the sFv to form the desired structure for target binding.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules.

The term “kit” describes a wide variety of bags, containers, carrying cases, and other portable enclosures which may be used to carry and store solid substances, liquid substances, and other accessories necessary to measure inflammatory genes and/or biomarkers of glioblastoma.

As used herein, “monitoring” refers to the actions of observing and checking the progress or quality of a treatment or procedure over a period of time. “Monitoring” also refers to observing the course of a disease or condition, such as a cancer, over a period of time.

A “gene” refers to a polynucleotide containing at least one open reading frame that is capable of encoding a particular polypeptide or protein after being transcribed and translated. Any of the polynucleotides sequences described herein may be used to identify larger fragments or full-length coding sequences of the gene with which they are associated.

As used herein, “normal” refers to a standard of health where no disease, disorder, abnormal condition, mutation, or dysfunction exists within a particular subject or patient. “Normal” can also refer to an average or typical state or condition.

The term “cancer” is used to address any neoplastic disease and is not limited to epithelial neoplasms (surface and glandular cancers; such a squamous cancers or adenomas)). It is used here to describe both solid tumors and hematologic malignancies, including epithelial (surface and glandular) cancers, glial tumors (including gliomas and glioblastomas), soft tissue, and bone sarcomas, angiomas, mesothelioma, melanoma, lymphomas, leukemias and myeloma.

A “glioma” refers to a primary brain or spinal cord tumors affecting the glial cells that surround nerve endings in the brain and spinal cord. Glial cells function as a support to neurons, however dysregulated cellular growth can lead to glioma formation. Gliomas are categorized by grade wherein slower growing gliomas are referred to as “low grade” or Grade 1 or 2. Fast growing or aggressive gliomas are referred to as “high grade” or Grade 3 or 4. An example of a high-grade glioma is a glioblastoma, typically categorized as a Grade 4.

### Methods of Identifying Progressing Glioblastoma

Gliomas are the most common form of malignant tumor in the central nervous system. In addition, glioblastomas, aggressive high-grade gliomas, comprise the highest malignancy rate and account for almost half of all brain tumors. Given the aggressiveness of these tumors, surgical removal does not always promise complete remission. In addition, treatments, such as chemotherapy, is commonly followed by showing new areas of contrast enhancement in brain MR imaging that could indicate tumor recurrence (termed progressive disease (PD)) requiring further treatment, or a treatment reaction (termed pseudo-progressive disease (psPD)). There is currently a need to distinguish between the two outcomes to ensure proper treatment is administered, and unnecessary surgeries are avoided.

As used herein, a “progressive disease” or a “progressing disease” refers to a disease or physical ailment whose course in most cases is the worsening, growth, or spread of disease. This worsening will continue until serious debility, organ failure, or death occurs. Some progressive diseases can be halted and/or reversed by treatment (surgical, dietary, or lifestyle interventions). Some examples of progressive diseases include, but are not limited to various cancers, Huntington's disease, Alzheimer's disease, multiple sclerosis, and Parkinson's disease. The terms “pseudoprogressive disease,” a “pseudoprogressing disease,” or a “pseudo disease” commonly refers to a phenomenon in which an initial increase in tumor size is observed or new lesions appear, followed by a decrease in tumor burden. This phenomenon can benefit patients receiving immunotherapy but often leads to premature discontinuation of treatment owing to the false judgement of progression.

The present disclosure provides an inflammatory gene panel and methods for uses and manufacture thereof.

In one aspect, disclosed herein is a method for identifying a subject with progressing glioblastoma versus pseudo-progressing glioblastoma, the method comprising a) determining an expression status of one or more genes selected from 1000 genes or more isolated from a biological sample from the subject, b) using the expression status to determine an expression score relative to a normal subject, c) identifying a subject with an expression score greater than 25% as being at risk of having progressing glioblastoma relative to a normal subject, d) identifying a subject with an expression score less than 25% as being at risk of having pseudo-progressing glioblastoma relative to a normal subject, and e) treating the subject based on the expression score.

In some embodiments, an expression status of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, or more genes is selected from 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 1751, 1752, 1753, 1754, 1755, 1756, 1757, 1758, 1759, 1760, 1761, 1762, 1763, 1764, 1765, 1766, 1767, 1768, 1769, 1770, 1771, 1772, 1773, 1774, 1775, 1776, 1777, 1778, 1779, 1780, 1781, 1782, 1783, 1784, 1785, 1786, 1787, 1788, 1789, 1790, 1791, 1792, 1793, 1794, 1795, 1796, 1797, 1798, 1799, 1800, 1801, 1802, 1803, 1804, 1805, 1806, 1807, 1808, 1809, 1810, 1811, 1812, 1813, 1814, 1815, 1816, 1817, 1818, 1819, 1820, 1821, 1822, 1823, 1824, 1825, 1826, 1827, 1828, 1829, 1830, 1831, 1832, 1833, 1834, 1835, 1836, 1837, 1838, 1839, 1840, 1841, 1842, 1843, 1844, 1845, 1846, 1847, 1848, 1849, 1850, 1851, 1852, 1853, 1854, 1855, 1856, 1857, 1858, 1859, 1860, 1861, 1862, 1863, 1864, 1865, 1866, 1867, 1868, 1869, 1870, 1871, 1872, 1873, 1874, 1875, 1876, 1877, 1878, 1879, 1880, 1881, 1882, 1883, 1884, 1885, 1886, 1887, 1888, 1889, 1890, 1891, 1892, 1893, 1894, 1895, 1896, 1897, 1898, 1899, 1900, 1901, 1902, 1903, 1904, 1905, 1906, 1907, 1908, 1909, 1910, 1911, 1912, 1913, 1914, 1915, 1916, 1917, 1918, 1919, 1920, 1921, 1922, 1923, 1924, 1925, 1926, 1927, 1928, 1929, 1930, 1931, 1932, 1933, 1934, 1935, 1936, 1937, 1938, 1939, 1940, 1941, 1942, 1943, 1944, 1945, 1946, 1947, 1948, 1949, 1950, 1951, 1952, 1953, 1954, 1955, 1956, 1957, 1958, 1959, 1960, 1961, 1962, 1963, 1964, 1965, 1966, 1967, 1968, 1969, 1970, 1971, 1972, 1973, 1974, 1975, 1976, 1977, 1978, 1979, 1980, 1981, 1982, 1983, 1984, 1985, 1986, 1987, 1988, 1989, 1990, 1991, 1992, 1993, 1994, 1995, 1996, 1997, 1998, 1999, 2000, 2001, 2002, 2003, 2004, 2005, 2006, 2007, 2008, 2009, 2010, 2011, 2012, 2013, 2014, 2015, 2016, 2017, 2018, 2019, 2020, 2021, 2022, 2023, 2024, 2025, 2026, 2027, 2028, 2029, 2030, 2031, 2032, 2033, 2034, 2035, 2036, 2037, 2038, 2039, 2040, 2041, 2042, 2043, 2044, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2097, 2098, 2099, 2100, 2101, 2102, 2103, 2104, 2105, 2106, 2107, 2108, 2109, 2110, 2111, 2112, 2113, 2114, 2115, 2116, 2117, 2118, 2119, 2120, 2121, 2122, 2123, 2124, 2125, 2126, 2127, 2128, 2129, 2130, 2131, 2132, 2133, 2134, 2135, 2136, 2137, 2138, 2139, 2140, 2141, 2142, 2143, 2144, 2145, 2146, 2147, 2148, 2149, 2150, 2151, 2152, 2153, 2154, 2155, 2156, 2157, 2158, 2159, 2160, 2161, 2162, 2163, 2164, 2165, 2166, 2167, 2168, 2169, 2170, 2171, 2172, 2173, 2174, 2175, 2176, 2177, 2178, 2179, 2180, 2181, 2182, 2183, 2184, 2185, 2186, 2187, 2188, 2189, 2190, 2191, 2192, 2193, 2194, 2195, 2196, 2197, 2198, 2199, 2200, 2201, 2202, 2203, 2204, 2205, 2206, 2207, 2208, 2209, 2210, 2211, 2212, 2213, 2214, 2215, 2216, 2217, 2218, 2219, 2220, 2221, 2222, 2223, 2224, 2225, 2226, 2227, 2228, 2229, 2230, 2231, 2232, 2233, 2234, 2235, 2236, 2237, 2238, 2239, 2240, 2241, 2242, 2243, 2244, 2245, 2246, 2247, 2248, 2249, 2250, 2251, 2252, 2253, 2254, 2255, 2256, 2257, 2258, 2259, 2260, 2261, 2262, 2263, 2264, 2265, 2266, 2267, 2268, 2269, 2270, 2271, 2272, 2273, 2274, 2275, 2276, 2277, 2278, 2279, 2280, 2281, 2282, 2283, 2284, 2285, 2286, 2287, 2288, 2289, 2290, 2291, 2292, 2293, 2294, 2295, 2296, 2297, 2298, 2299, 2300, 2301, 2302, 2303, 2304, 2305, 2306, 2307, 2308, 2309, 2310, 2311, 2312, 2313, 2314, 2315, 2316, 2317, 2318, 2319, 2320, 2321, 2322, 2323, 2324, 2325, 2326, 2327, 2328, 2329, 2330, 2331, 2332, 2333, 2334, 2335, 2336, 2337, 2338, 2339, 2340, 2341, 2342, 2343, 2344, 2345, 2346, 2347, 2348, 2349, 2350, 2351, 2352, 2353, 2354, 2355, 2356, 2357, 2358, 2359, 2360, 2361, 2362, 2363, 2364, 2365, 2366, 2367, 2368, 2369, 2370, 2371, 2372, 2373, 2374, 2375, 2376, 2377, 2378, 2379, 2380, 2381, 2382, 2383, 2384, 2385, 2386, 2387, 2388, 2389, 2390, 2391, 2392, 2393, 2394, 2395, 2396, 2397, 2398, 2399, 2400, 2401, 2402, 2403, 2404, 2405, 2406, 2407, 2408, 2409, 2410, 2411, 2412, 2413, 2414, 2415, 2416, 2417, 2418, 2419, 2420, 2421, 2422, 2423, 2424, 2425, 2426, 2427, 2428, 2429, 2430, 2431, 2432, 2433, 2434, 2435, 2436, 2437, 2438, 2439, 2440, 2441, 2442, 2443, 2444, 2445, 2446, 2447, 2448, 2449, 2450, 2451, 2452, 2453, 2454, 2455, 2456, 2457, 2458, 2459, 2460, 2461, 2462, 2463, 2464, 2465, 2466, 2467, 2468, 2469, 2470, 2471, 2472, 2473, 2474, 2475, 2476, 2477, 2478, 2479, 2480, 2481, 2482, 2483, 2484, 2485, 2486, 2487, 2488, 2489, 2490, 2491, 2492, 2493, 2494, 2495, 2496, 2497, 2498, 2499, 2500, 2501, 2502, 2503, 2504, 2505, 2506, 2507, 2508, 2509, 2510, 2511, 2512, 2513, 2514, 2515, 2516, 2517, 2518, 2519, 2520, 2521, 2522, 2523, 2524, 2525, 2526, 2527, 2528, 2529, 2530, 2531, 2532, 2533, 2534, 2535, 2536, 2537, 2538, 2539, 2540, 2541, 2542, 2543, 2544, 2545, 2546, 2547, 2548, 2549, 2550, 2551, 2552, 2553, 2554, 2555, 2556, 2557, 2558, 2559, 2560, 2561, 2562, 2563, 2564, 2565, 2566, 2567, 2568, 2569, 2570, 2571, 2572, 2573, 2574, 2575, 2576, 2577, 2578, 2579, 2580, 2581, 2582, 2583, 2584, 2585, 2586, 2587, 2588, 2589, 2590, 2591, 2592, 2593, 2594, 2595, 2596, 2597, 2598, 2599, 2600, 2601, 2602, 2603, 2604, 2605, 2606, 2607, 2608, 2609, 2610, 2611, 2612, 2613, 2614, 2615, 2616, 2617, 2618, 2619, 2620, 2621, 2622, 2623, 2624, 2625, 2626, 2627, 2628, 2629, 2630, 2631, 2632, 2633, 2634, 2635, 2636, 2637, 2638, 2639, 2640, 2641, 2642, 2643, 2644, 2645, 2646, 2647, 2648, 2649, 2650, 2651, 2652, 2653, 2654, 2655, 2656, 2657, 2658, 2659, 2660, 2661, 2662, 2663, 2664, 2665, 2666, 2667, 2668, 2669, 2670, 2671, 2672, 2673, 2674, 2675, 2676, 2677, 2678, 2679, 2680, 2681, 2682, 2683, 2684, 2685, 2686, 2687, 2688, 2689, 2690, 2691, 2692, 2693, 2694, 2695, 2696, 2697, 2698, 2699, 2700, 2701, 2702, 2703, 2704, 2705, 2706, 2707, 2708, 2709, 2710, 2711, 2712, 2713, 2714, 2715, 2716, 2717, 2718, 2719, 2720, 2721, 2722, 2723, 2724, 2725, 2726, 2727, 2728, 2729, 2730, 2731, 2732, 2733, 2734, 2735, 2736, 2737, 2738, 2739, 2740, 2741, 2742, 2743, 2744, 2745, 2746, 2747, 2748, 2749, 2750, 2751, 2752, 2753, 2754, 2755, 2756, 2757, 2758, 2759, 2760, 2761, 2762, 2763, 2764, 2765, 2766, 2767, 2768, 2769, 2770, 2771, 2772, 2773, 2774, 2775, 2776, 2777, 2778, 2779, 2780, 2781, 2782, 2783, 2784, 2785, 2786, 2787, 2788, 2789, 2790, 2791, 2792, 2793, 2794, 2795, 2796, 2797, 2798, 2799, 2800, 2801, 2802, 2803, 2804, 2805, 2806, 2807, 2808, 2809, 2810, 2811, 2812, 2813, 2814, 2815, 2816, 2817, 2818, 2819, 2820, 2821, 2822, 2823, 2824, 2825, 2826, 2827, 2828, 2829, 2830, 2831, 2832, 2833, 2834, 2835, 2836, 2837, 2838, 2839, 2840, 2841, 2842, 2843, 2844, 2845, 2846, 2847, 2848, 2849, 2850, 2851, 2852, 2853, 2854, 2855, 2856, 2857, 2858, 2859, 2860, 2861, 2862, 2863, 2864, 2865, 2866, 2867, 2868, 2869, 2870, 2871, 2872, 2873, 2874, 2875, 2876, 2877, 2878, 2879, 2880, 2881, 2882, 2883, 2884, 2885, 2886, 2887, 2888, 2889, 2890, 2891, 2892, 2893, 2894, 2895, 2896, 2897, 2898, 2899, 2900, 2901, 2902, 2903, 2904, 2905, 2906, 2907, 2908, 2909, 2910, 2911, 2912, 2913, 2914, 2915, 2916, 2917, 2918, 2919, 2920, 2921, 2922, 2923, 2924, 2925, 2926, 2927, 2928, 2929, 2930, 2931, 2932, 2933, 2934, 2935, 2936, 2937, 2938, 2939, 2940, 2941, 2942, 2943, 2944, 2945, 2946, 2947, 2948, 2949, 2950, 2951, 2952, 2953, 2954, 2955, 2956, 2957, 2958, 2959, 2960, 2961, 2962, 2963, 2964, 2965, 2966, 2967, 2968, 2969, 2970, 2971, 2972, 2973, 2974, 2975, 2976, 2977, 2978, 2979, 2980, 2981, 2982, 2983, 2984, 2985, 2986, 2987, 2988, 2989, 2990, 2991, 2992, 2993, 2994, 2995, 2996, 2997, 2998, 2999, 3000, 3001, 3002, 3003, 3004, 3005, 3006, 3007, 3008, 3009, 3010, 3011, 3012, 3013, 3014, 3015, 3016, 3017, 3018, 3019, 3020, 3021, 3022, 3023, 3024, 3025, 3026, 3027, 3028, 3029, 3030, 3031, 3032, 3033, 3034, 3035, 3036, 3037, 3038, 3039, 3040, 3041, 3042, 3043, 3044, 3045, 3046, 3047, 3048, 3049, 3050, 3051, 3052, 3053, 3054, 3055, 3056, 3057, 3058, 3059, 3060, 3061, 3062, 3063, 3064, 3065, 3066, 3067, 3068, 3069, 3070, 3071, 3072, 3073, 3074, 3075, 3076, 3077, 3078, 3079, 3080, 3081, 3082, 3083, 3084, 3085, 3086, 3087, 3088, 3089, 3090, 3091, 3092, 3093, 3094, 3095, 3096, 3097, 3098, 3099, 3100, 3101, 3102, 3103, 3104, 3105, 3106, 3107, 3108, 3109, 3110, 3111, 3112, 3113, 3114, 3115, 3116, 3117, 3118, 3119, 3120, 3121, 3122, 3123, 3124, 3125, 3126, 3127, 3128, 3129, 3130, 3131, 3132, 3133, 3134, 3135, 3136, 3137, 3138, 3139, 3140, 3141, 3142, 3143, 3144, 3145, 3146, 3147, 3148, 3149, 3150, 3151, 3152, 3153, 3154, 3155, 3156, 3157, 3158, 3159, 3160, 3161, 3162, 3163, 3164, 3165, 3166, 3167, 3168, 3169, 3170, 3171, 3172, 3173, 3174, 3175, 3176, 3177, 3178, 3179, 3180, 3181, 3182, 3183, 3184, 3185, 3186, 3187, 3188, 3189, 3190, 3191, 3192, 3193, 3194, 3195, 3196, 3197, 3198, 3199, 3200, 3201, 3202, 3203, 3204, 3205, 3206, 3207, 3208, 3209, 3210, 3211, 3212, 3213, 3214, 3215, 3216, 3217, 3218, 3219, 3220, 3221, 3222, 3223, 3224, 3225, 3226, 3227, 3228, 3229, 3230, 3231, 3232, 3233, 3234, 3235, 3236, 3237, 3238, 3239, 3240, 3241, 3242, 3243, 3244, 3245, 3246, 3247, 3248, 3249, 3250, 3251, 3252, 3253, 3254, 3255, 3256, 3257, 3258, 3259, 3260, 3261, 3262, 3263, 3264, 3265, 3266, 3267, 3268, 3269, 3270, 3271, 3272, 3273, 3274, 3275, 3276, 3277, 3278, 3279, 3280, 3281, 3282, 3283, 3284, 3285, 3286, 3287, 3288, 3289, 3290, 3291, 3292, 3293, 3294, 3295, 3296, 3297, 3298, 3299, 3300, 3301, 3302, 3303, 3304, 3305, 3306, 3307, 3308, 3309, 3310, 3311, 3312, 3313, 3314, 3315, 3316, 3317, 3318, 3319, 3320, 3321, 3322, 3323, 3324, 3325, 3326, 3327, 3328, 3329, 3330, 3331, 3332, 3333, 3334, 3335, 3336, 3337, 3338, 3339, 3340, 3341, 3342, 3343, 3344, 3345, 3346, 3347, 3348, 3349, 3350, 3351, 3352, 3353, 3354, 3355, 3356, 3357, 3358, 3359, 3360, 3361, 3362, 3363, 3364, 3365, 3366, 3367, 3368, 3369, 3370, 3371, 3372, 3373, 3374, 3375, 3376, 3377, 3378, 3379, 3380, 3381, 3382, 3383, 3384, 3385, 3386, 3387, 3388, 3389, 3390, 3391, 3392, 3393, 3394, 3395, 3396, 3397, 3398, 3399, 3400, 3401, 3402, 3403, 3404, 3405, 3406, 3407, 3408, 3409, 3410, 3411, 3412, 3413, 3414, 3415, 3416, 3417, 3418, 3419, 3420, 3421, 3422, 3423, 3424, 3425, 3426, 3427, 3428, 3429, 3430, 3431, 3432, 3433, 3434, 3435, 3436, 3437, 3438, 3439, 3440, 3441, 3442, 3443, 3444, 3445, 3446, 3447, 3448, 3449, 3450, 3451, 3452, 3453, 3454, 3455, 3456, 3457, 3458, 3459, 3460, 3461, 3462, 3463, 3464, 3465, 3466, 3467, 3468, 3469, 3470, 3471, 3472, 3473, 3474, 3475, 3476, 3477, 3478, 3479, 3480, 3481, 3482, 3483, 3484, 3485, 3486, 3487, 3488, 3489, 3490, 3491, 3492, 3493, 3494, 3495, 3496, 3497, 3498, 3499, 3500, 3501, 3502, 3503, 3504, 3505, 3506, 3507, 3508, 3509, 3510, 3511, 3512, 3513, 3514, 3515, 3516, 3517, 3518, 3519, 3520, 3521, 3522, 3523, 3524, 3525, 3526, 3527, 3528, 3529, 3530, 3531, 3532, 3533, 3534, 3535, 3536, 3537, 3538, 3539, 3540, 3541, 3542, 3543, 3544, 3545, 3546, 3547, 3548, 3549, 3550, 3551, 3552, 3553, 3554, 3555, 3556, 3557, 3558, 3559, 3560, 3561, 3562, 3563, 3564, 3565, 3566, 3567, 3568, 3569, 3570, 3571, 3572, 3573, 3574, 3575, 3576, 3577, 3578, 3579, 3580, 3581, 3582, 3583, 3584, 3585, 3586, 3587, 3588, 3589, 3590, 3591, 3592, 3593, 3594, 3595, 3596, 3597, 3598, 3599, 3600, 3601, 3602, 3603, 3604, 3605, 3606, 3607, 3608, 3609, 3610, 3611, 3612, 3613, 3614, 3615, 3616, 3617, 3618, 3619, 3620, 3621, 3622, 3623, 3624, 3625, 3626, 3627, 3628, 3629, 3630, 3631, 3632, 3633, 3634, 3635, 3636, 3637, 3638, 3639, 3640, 3641, 3642, 3643, 3644, 3645, 3646, 3647, 3648, 3649, 3650, 3651, 3652, 3653, 3654, 3655, 3656, 3657, 3658, 3659, 3660, 3661, 3662, 3663, 3664, 3665, 3666, 3667, 3668, 3669, 3670, 3671, 3672, 3673, 3674, 3675, 3676, 3677, 3678, 3679, 3680, 3681, 3682, 3683, 3684, 3685, 3686, 3687, 3688, 3689, 3690, 3691, 3692, 3693, 3694, 3695, 3696, 3697, 3698, 3699, 3700, 3701, 3702, 3703, 3704, 3705, 3706, 3707, 3708, 3709, 3710, 3711, 3712, 3713, 3714, 3715, 3716, 3717, 3718, 3719, 3720, 3721, 3722, 3723, 3724, 3725, 3726, 3727, 3728, 3729, 3730, 3731, 3732, 3733, 3734, 3735, 3736, 3737, 3738, 3739, 3740, 3741, 3742, 3743, 3744, 3745, 3746, 3747, 3748, 3749, 3750, 3751, 3752, 3753, 3754, 3755, 3756, 3757, 3758, 3759, 3760, 3761, 3762, 3763, 3764, 3765, 3766, 3767, 3768, 3769, 3770, 3771, 3772, 3773, 3774, 3775, 3776, 3777, 3778, 3779, 3780, 3781, 3782, 3783, 3784, 3785, 3786, 3787, 3788, 3789, 3790, 3791, 3792, 3793, 3794, 3795, 3796, 3797, 3798, 3799, 3800, 3801, 3802, 3803, 3804, 3805, 3806, 3807, 3808, 3809, 3810, 3811, 3812, 3813, 3814, 3815, 3816, 3817, 3818, 3819, 3820, 3821, 3822, 3823, 3824, 3825, 3826, 3827, 3828, 3829, 3830, 3831, 3832, 3833, 3834, 3835, 3836, 3837, 3838, 3839, 3840, 3841, 3842, 3843, 3844, 3845, 3846, 3847, 3848, 3849, 3850, 3851, 3852, 3853, 3854, 3855, 3856, 3857, 3858, 3859, 3860, 3861, 3862, 3863, 3864, 3865, 3866, 3867, 3868, 3869, 3870, 3871, 3872, 3873, 3874, 3875, 3876, 3877, 3878, 3879, 3880, 3881, 3882, 3883, 3884, 3885, 3886, 3887, 3888, 3889, 3890, 3891, 3892, 3893, 3894, 3895, 3896, 3897, 3898, 3899, 3900, 3901, 3902, 3903, 3904, 3905, 3906, 3907, 3908, 3909, 3910, 3911, 3912, 3913, 3914, 3915, 3916, 3917, 3918, 3919, 3920, 3921, 3922, 3923, 3924, 3925, 3926, 3927, 3928, 3929, 3930, 3931, 3932, 3933, 3934, 3935, 3936, 3937, 3938, 3939, 3940, 3941, 3942, 3943, 3944, 3945, 3946, 3947, 3948, 3949, 3950, 3951, 3952, 3953, 3954, 3955, 3956, 3957, 3958, 3959, 3960, 3961, 3962, 3963, 3964, 3965, 3966, 3967, 3968, 3969, 3970, 3971, 3972, 3973, 3974, 3975, 3976, 3977, 3978, 3979, 3980, 3981, 3982, 3983, 3984, 3985, 3986, 3987, 3988, 3989, 3990, 3991, 3992, 3993, 3994, 3995, 3996, 3997, 3998, 3999, 4000, 4001, 4002, 4003, 4004, 4005, 4006, 4007, 4008, 4009, 4010, 4011, 4012, 4013, 4014, 4015, 4016, 4017, 4018, 4019, 4020, 4021, 4022, 4023, 4024, 4025, 4026, 4027, 4028, 4029, 4030, 4031, 4032, 4033, 4034, 4035, 4036, 4037, 4038, 4039, 4040, 4041, 4042, 4043, 4044, 4045, 4046, 4047, 4048, 4049, 4050, 4051, 4052, 4053, 4054, 4055, 4056, 4057, 4058, 4059, 4060, 4061, 4062, 4063, 4064, 4065, 4066, 4067, 4068, 4069, 4070, 4071, 4072, 4073, 4074, 4075, 4076, 4077, 4078, 4079, 4080, 4081, 4082, 4083, 4084, 4085, 4086, 4087, 4088, 4089, 4090, 4091, 4092, 4093, 4094, 4095, 4096, 4097, 4098, 4099, 4100, 4101, 4102, 4103, 4104, 4105, 4106, 4107, 4108, 4109, 4110, 4111, 4112, 4113, 4114, 4115, 4116, 4117, 4118, 4119, 4120, 4121, 4122, 4123, 4124, 4125, 4126, 4127, 4128, 4129, 4130, 4131, 4132, 4133, 4134, 4135, 4136, 4137, 4138, 4139, 4140, 4141, 4142, 4143, 4144, 4145, 4146, 4147, 4148, 4149, 4150, 4151, 4152, 4153, 4154, 4155, 4156, 4157, 4158, 4159, 4160, 4161, 4162, 4163, 4164, 4165, 4166, 4167, 4168, 4169, 4170, 4171, 4172, 4173, 4174, 4175, 4176, 4177, 4178, 4179, 4180, 4181, 4182, 4183, 4184, 4185, 4186, 4187, 4188, 4189, 4190, 4191, 4192, 4193, 4194, 4195, 4196, 4197, 4198, 4199, 4200, 4201, 4202, 4203, 4204, 4205, 4206, 4207, 4208, 4209, 4210, 4211, 4212, 4213, 4214, 4215, 4216, 4217, 4218, 4219, 4220, 4221, 4222, 4223, 4224, 4225, 4226, 4227, 4228, 4229, 4230, 4231, 4232, 4233, 4234, 4235, 4236, 4237, 4238, 4239, 4240, 4241, 4242, 4243, 4244, 4245, 4246, 4247, 4248, 4249, 4250, 4251, 4252, 4253, 4254, 4255, 4256, 4257, 4258, 4259, 4260, 4261, 4262, 4263, 4264, 4265, 4266, 4267, 4268, 4269, 4270, 4271, 4272, 4273, 4274, 4275, 4276, 4277, 4278, 4279, 4280, 4281, 4282, 4283, 4284, 4285, 4286, 4287, 4288, 4289, 4290, 4291, 4292, 4293, 4294, 4295, 4296, 4297, 4298, 4299, 4300, 4301, 4302, 4303, 4304, 4305, 4306, 4307, 4308, 4309, 4310, 4311, 4312, 4313, 4314, 4315, 4316, 4317, 4318, 4319, 4320, 4321, 4322, 4323, 4324, 4325, 4326, 4327, 4328, 4329, 4330, 4331, 4332, 4333, 4334, 4335, 4336, 4337, 4338, 4339, 4340, 4341, 4342, 4343, 4344, 4345, 4346, 4347, 4348, 4349, 4350, 4351, 4352, 4353, 4354, 4355, 4356, 4357, 4358, 4359, 4360, 4361, 4362, 4363, 4364, 4365, 4366, 4367, 4368, 4369, 4370, 4371, 4372, 4373, 4374, 4375, 4376, 4377, 4378, 4379, 4380, 4381, 4382, 4383, 4384, 4385, 4386, 4387, 4388, 4389, 4390, 4391, 4392, 4393, 4394, 4395, 4396, 4397, 4398, 4399, 4400, 4401, 4402, 4403, 4404, 4405, 4406, 4407, 4408, 4409, 4410, 4411, 4412, 4413, 4414, 4415, 4416, 4417, 4418, 4419, 4420, 4421, 4422, 4423, 4424, 4425, 4426, 4427, 4428, 4429, 4430, 4431, 4432, 4433, 4434, 4435, 4436, 4437, 4438, 4439, 4440, 4441, 4442, 4443, 4444, 4445, 4446, 4447, 4448, 4449, 4450, 4451, 4452, 4453, 4454, 4455, 4456, 4457, 4458, 4459, 4460, 4461, 4462, 4463, 4464, 4465, 4466, 4467, 4468, 4469, 4470, 4471, 4472, 4473, 4474, 4475, 4476, 4477, 4478, 4479, 4480, 4481, 4482, 4483, 4484, 4485, 4486, 4487, 4488, 4489, 4490, 4491, 4492, 4493, 4494, 4495, 4496, 4497, 4498, 4499, 4500, 4501, 4502, 4503, 4504, 4505, 4506, 4507, 4508, 4509, 4510, 4511, 4512, 4513, 4514, 4515, 4516, 4517, 4518, 4519, 4520, 4521, 4522, 4523, 4524, 4525, 4526, 4527, 4528, 4529, 4530, 4531, 4532, 4533, 4534, 4535, 4536, 4537, 4538, 4539, 4540, 4541, 4542, 4543, 4544, 4545, 4546, 4547, 4548, 4549, 4550, 4551, 4552, 4553, 4554, 4555, 4556, 4557, 4558, 4559, 4560, 4561, 4562, 4563, 4564, 4565, 4566, 4567, 4568, 4569, 4570, 4571, 4572, 4573, 4574, 4575, 4576, 4577, 4578, 4579, 4580, 4581, 4582, 4583, 4584, 4585, 4586, 4587, 4588, 4589, 4590, 4591, 4592, 4593, 4594, 4595, 4596, 4597, 4598, 4599, 4600, 4601, 4602, 4603, 4604, 4605, 4606, 4607, 4608, 4609, 4610, 4611, 4612, 4613, 4614, 4615, 4616, 4617, 4618, 4619, 4620, 4621, 4622, 4623, 4624, 4625, 4626, 4627, 4628, 4629, 4630, 4631, 4632, 4633, 4634, 4635, 4636, 4637, 4638, 4639, 4640, 4641, 4642, 4643, 4644, 4645, 4646, 4647, 4648, 4649, 4650, 4651, 4652, 4653, 4654, 4655, 4656, 4657, 4658, 4659, 4660, 4661, 4662, 4663, 4664, 4665, 4666, 4667, 4668, 4669, 4670, 4671, 4672, 4673, 4674, 4675, 4676, 4677, 4678, 4679, 4680, 4681, 4682, 4683, 4684, 4685, 4686, 4687, 4688, 4689, 4690, 4691, 4692, 4693, 4694, 4695, 4696, 4697, 4698, 4699, 4700, 4701, 4702, 4703, 4704, 4705, 4706, 4707, 4708, 4709, 4710, 4711, 4712, 4713, 4714, 4715, 4716, 4717, 4718, 4719, 4720, 4721, 4722, 4723, 4724, 4725, 4726, 4727, 4728, 4729, 4730, 4731, 4732, 4733, 4734, 4735, 4736, 4737, 4738, 4739, 4740, 4741, 4742, 4743, 4744, 4745, 4746, 4747, 4748, 4749, 4750, 4751, 4752, 4753, 4754, 4755, 4756, 4757, 4758, 4759, 4760, 4761, 4762, 4763, 4764, 4765, 4766, 4767, 4768, 4769, 4770, 4771, 4772, 4773, 4774, 4775, 4776, 4777, 4778, 4779, 4780, 4781, 4782, 4783, 4784, 4785, 4786, 4787, 4788, 4789, 4790, 4791, 4792, 4793, 4794, 4795, 4796, 4797, 4798, 4799, 4800, 4801, 4802, 4803, 4804, 4805, 4806, 4807, 4808, 4809, 4810, 4811, 4812, 4813, 4814, 4815, 4816, 4817, 4818, 4819, 4820, 4821, 4822, 4823, 4824, 4825, 4826, 4827, 4828, 4829, 4830, 4831, 4832, 4833, 4834, 4835, 4836, 4837, 4838, 4839, 4840, 4841, 4842, 4843, 4844, 4845, 4846, 4847, 4848, 4849, 4850, 4851, 4852, 4853, 4854, 4855, 4856, 4857, 4858, 4859, 4860, 4861, 4862, 4863, 4864, 4865, 4866, 4867, 4868, 4869, 4870, 4871, 4872, 4873, 4874, 4875, 4876, 4877, 4878, 4879, 4880, 4881, 4882, 4883, 4884, 4885, 4886, 4887, 4888, 4889, 4890, 4891, 4892, 4893, 4894, 4895, 4896, 4897, 4898, 4899, 4900, 4901, 4902, 4903, 4904, 4905, 4906, 4907, 4908, 4909, 4910, 4911, 4912, 4913, 4914, 4915, 4916, 4917, 4918, 4919, 4920, 4921, 4922, 4923, 4924, 4925, 4926, 4927, 4928, 4929, 4930, 4931, 4932, 4933, 4934, 4935, 4936, 4937, 4938, 4939, 4940, 4941, 4942, 4943, 4944, 4945, 4946, 4947, 4948, 4949, 4950, 4951, 4952, 4953, 4954, 4955, 4956, 4957, 4958, 4959, 4960, 4961, 4962, 4963, 4964, 4965, 4966, 4967, 4968, 4969, 4970, 4971, 4972, 4973, 4974, 4975, 4976, 4977, 4978, 4979, 4980, 4981, 4982, 4983, 4984, 4985, 4986, 4987, 4988, 4989, 4990, 4991, 4992, 4993, 4994, 4995, 4996, 4997, 4998, 4999, 5000, or more genes isolated from a biological sample. In another embodiment, the subject is identified with an expression score of 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as being at risk of having progressing glioblastoma relative to a normal subject. In another embodiment, the subject is identified with an expression score of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, or 24% as being at risk of having pseudo-progressing glioblastoma relative to a normal subject.

In another embodiment, the expression status is determined from any one gene including but not limited to AC005592.2, AC007879.7, ACTA2, ACTG2, ADAM8, ADM, ANGPTL4, ANKRD1, ANPEP, AOC3, APCDD1L, ATF5, ATP2A3, ATP6VOD2, B3GNT7, BCL3, C15orf48, CA9, CCIN, CCR3, CCR7, CD1C, CD300E, CLCF1, CLDN23, CNN2, COL1A1, COL1A2, COL3A1, COL5A1, COL6A3, CSTA, CXCR4, CYR61, DMBX1, DPP4, EGLN3, EMILIN2, FAM46B, FN1, FOSL1, FOSL2, FPR2, GALNT5, GPR18, HBEGF, HOXB6, HOXB7, ICAM1, IL1R1, IL7R, IL8, IRF1, ITK, KLF4, LCK, LIF, LOX, LUCAT1, MAFF, MAPK13, MEDAG, MEFV, MMP11, MMP9, NABP1, NAMPT, NDRG1, NFKBIA, NLRP12, P4HA3, PAX8, PDLIM1, PIM1, PLAUR, PLEKHS1, PPP1R15A, PTGER2, RAB43, RAC2, RGS1, RGS2, RHOH, RND3, ROR2, RP11-1149023.2, RP11-1149023.3, RP11-290F20.3, RP11-59D5_B.2, RP5-1021120.2, S100A8, S100A9, SDC1, SERPINE1, SLC16A3, SNAIL, SPOCD1, SUSD2, THBD, THBS1, TMEM217, TNFAIP6, TNFAIP8, TNFRSF10D, TREM1, TREML2, TREML3P, VDR, C3H12A, ABCA6, AC002059.10, AC005932.1, AC008060.7, AC011558.5, AC017076.5, AC098823.3, ACSM5, ACTA2-AS1, ADRA2B, APLNR, ARHGEF5, ARRDC2, BEST1, BHLHE41, CD55, CD83, CDK18, CLCA2, CLMN, COL4A5, CPXM2, CTC-1337H24.1, CTD-2034121.2, CXorf65, CYB5R2, DNM1P47, DYSF, EHF, ELOVL1, EVI2A, FAM124A, FAM53B, FGF20, FKBP11, FMN1, GALNT6, GPR37, GTSF1, HMSD, HOXD-AS1, HPN-AS1, ICOSLG, IL9R, IP6K3, KANK4, KCNJ2-AS1, KCNN4, KEL, LAG3, LHB, LINC01010, LRMP, LRP2, LRRC63, LURAP1, MFSD6L, MGST1, MIR24-2, MS4A4E, NFKBID, NME8, OSTF1, P2RX1, PATL2, PCP2, PCSK6, PDLIM7, PLA2G7, PLD1, PM20D1, PPM1N, PRG4, RP11-1094M14.8, RP11-136K7.2, RP11-161H23.5, RP11-16K12.1, RP11-350G8.5, RP11-399019.9, RP11-434H6.6, RP11-597D13.7. RP11-700H6.1, RP11-703116.1, RP11-883G14.1, RP3-47704.14, RP5-943J3.1, RP6-99M1.2, RPS10P7, SAMD3, SEPP1, SH2D3A, SLC45A3, SLCS2A1, ST18, SVIP, TM6SF1, TMC7, TMEM139, TMPRSS13, TPM2, TRIM59, TRPC2, TRPM6, TRPV5, U91328.20, UCN2, ABCG1, ABI3BP, AC003003.5, AC004988.1, AC005082.12, AC006129.1, AC006129.2, AC011899.9, AC026202.3, ACAT2, ACP5, ACSL1, ADAM28, ADAMTS1, ADAMTS12, ADAP2, ADRB2, AF127936.5, AKR1B10, AKR1B15, ALOX12B, ALOX5, ALOX5AP, AMICA1, AMPD3, ANKRD22, AOAH, AP000476.1, AP000783.1, APBB1IP, APH1B, APOBEC3C, APOBR, ARHGAP30, ARHGDIB, ARHGEF34P, ARHGEF35, ARL11, ARL3, ARRDC4, ASB12, ATP8B4, AXL, B4GALT1, BCL2A1, BCR, BIN2, BIRC3, BMP2K, BTK, C10orf10, C11orf21, C11orf45, C16orf54, C16orf92, C19orf35, C1QC, C2, C3AR1, C4BPB, C5AR1, C5AR2, C9orf47, C9orf66, CABP7, CACNA2D4, CASP1, CASP10, CASP5, CASS4, CBFA2T3, CCL18, CCL2, CCL20, CCL22, CCL8, CCR1, CCR2, CCR4, CCR5, CCRL2, CD14, CD163, CD180, CD1D, CD200R1, CD274, CD28, CD300A, CD300C, CD300LB, CD300LF, CD3G, CD4, CD40, CD40LG, CD53, CD59, CD68, CD69, CD80, CD84, CD86, CD96, CDCP1, CDKN1A, CEACAM21, CEACAM4, CEBPA, CECR1, CELSR3, CFH, CFLAR, CHCHD7, CHRM3-AS2, CIITA, CLEC10A, CLEC12A, CLEC12B, CLEC2B, CLEC7A, CLSTN3, CMAHP, CMKLR1, CMPK2, CNN1, COMP, COQ3, CP, CPA6, CPM, CR1, CREG1, CRTAM, CSF1R, CSF2RB, CSF3R, CTC-205M6.5, CTC-250114.6, CTD-2258A20.5, CTD-2306M10.1, CTD-2540B15.7, CTD-2587H19.1, CTSB, CTSC, CTSH, CTSL, CTSS, CTSZ, CXCL12, CXCL16, CXCL6, CXCR2P1, CXCR6, CXorf21, CYBB, CYP19A1, CYTH4, CYTIP, DAB2, DAPP1, DCN, DCSTAMP, DDO, DDX58, DEC1, DENND3, DLGAP1-AS3, DLX2, DLX6, DLX6-AS1, DMRTC1B, DNAJC5B, DNASE2B, DOCK2, DOCK8, DOK3, DPY19L2P4, DRAM1, DSC2, DUSP1, EFCAB4B, ELL2, EMB, EMR1, EMR2, EMR4P, EPSTI1, EVI2B, F13A1, FAM105A FAM177B, FAM20A, FAM213A, FAM57B, FASLG, FAXC, FBP1, FBXO32, FCER1A, FCER1G, FCGBP, FCGR2A, FCGR2B, FCGR3A, FCRL6, FFAR4, FGD2, FGL2, FGR, FH, FLVCR2, FMO2, FMO4, FMOD, FOXD4, FPR1, FPR3, FSD1, FUCA1, FUT9, GAB3, GADD45B, GAL3ST4, GALNT12, GAPT, GATA3, GBP1P1, GBP2, GBP3, GCH1, GCNT1, GDAP1L1, GDI1, GIMAP2, GIMAP4, GIMAP6, GIMAP7, GIMAP8, GLIPR1, GLRX, GM2A, GNA15, GPNMB, GPR132, GPR141, GPR160, GPR183, GPR34, GPR65, GPR82, GPR84, GPR85, GPRIN3, GRM8, GSK3A, GVINP1, GVQW1, HAVCR2, HCK, HCLS1, HCN4, HLA-DMA, HLA-DMB, HLA-DOA, HLA-DPA1, HLA-DQA2, HLA-DRA, HNMT, HPGDS, HPSE, HTR2B, HTRA4, IFI30, IFI44L, IFIH1, IFIT3, IGF1, IGSF6, IKBKE, IKZF1, IL10, IL10RA, IL12RB1, IL15RA, IL16, IL17RA, IL18BP, IL1A, IL1B, IL1RN, IL21R, IL2RA, IL2RB, IL2RG, IL4I1, IL4R, IL6, ILDR1, IRAK3, IRF4, IRF5, IRF8, IRF9, ITGAL, ITGAM, ITGAX, ITGB2, JPH2, KCNF1, KCNJ5, KCNK13, KCNMB1, KCNQ1, KLC2, KLHDC7B, KLHL6, KLRC1, KLRD1, KMO, KYNU, LA16c-380H5.5, LACTB, LAIR1, LAMP1, LANCL2, LAPTMS, LAT2, LAYN, LBP, LCP1, LCP2, LGALS17A, LILRA1, LILRA6, LILRB1, LILRB2, LILRB4, LILRB5, LINC00539, LINC00578, LINC00702, LINC00968, LPAR5, LPCAT2, LPXN, LRRC25, LRRC39, LRRTM1, LTBP2, LTBR, LUM, LURAP1L, LY9, LYN, LYSMD2, LYVE1, LYZ, M6PR, MAF, MAFB, MALL, MAN1A1, MAN1C1, MAOB, MAP3K8, MCOLN2, MERTK, MGAT4A, MGAT5B, MITF, MKNK1, MLKL, MLPH, MMD2, MNDA, MPEG1, MRC1, MRC1L1, MS4A4A, MS4A6A, MS4A7, MSR1, MX1, MX2, MYO1F, NAAA, NCF2, NCKAP1L, NDUFA6-AS1, NFAM1, NFATC2, NGFR, NGFRAP1, NLRC4, NPL, NRROS, NUDT16P, OAS1, OAS2, OLFML2B, OLFML3, OLR1, OSM, OTUD1, P2RY13, P2RY6, P2RY8, PALD1, PALMD, PAPPA, PARP14, PARP15, PCED1B, PDCD1LG2, PDZD4, PGA5, PIK3AP1, PIK3CD, PIK3R5, PIRT, PJA1, PLA2G15, PLB1, PLBD1, PLCG2, PLEK, PLEKHA3P1, PLEKHO2, PLXDC2, POU2F2, PPARG, PPP1R14C, PPP1R17, PRDM12, PRKCD, PRLR, PRRT4, PTAFR, PTCHD2, PTGDR, PTGER4, PTGFR, PTPN22, PTPN7, PTPRC, PTX3, PYHIN1, RAB20, RAB38, RAB44, RAD51B, RARRES1, RASGRP4, RASL11B, RBM47, RERGL, RGS18, RHBDF2, RHOBTB2, RIN3, RIPK3, RNASE2, RNASE3, RNF112, RNF144B, RP11-1024P17.1, RP11-10K16.1, RP11-1149M10.2, RP11-117D22.2, RP11-119F19.2, RP11-121A8.1, RP11-153M7.1, RP11-153M7.3, RP11-196G18.3, RP11-23P13.6, RP11-245J24.1, RP11-271K11.5, RP11-307C19.1, RP11-325F22.2, RP11-346C20.4, RP11-353N4.5, RP11-357K6.1, RP11-363E6.3, RP11-364B14.3, RP11-379B8.1, RP11-384L8.1, RP11-3B12.1, RP11-403P17.5, RP11-439A17.9. RP11-477J21.6, RP11-47122.3, RP11-497H16.7, RP11-523018.5, RP11-565J7.1, RP11-588H23.3, RP11-626G11.4, RP11-676J15.1, RP11-70C1.1, RP11-733018.1, RP11-750H9.5, RP11-760N9.1, RP11-792D21.2, RP11-838N2.4, RP11-84C10.2, RP11-861A13.4, RP11-90P13.1, RP11-93B14.5, RP11-960L18.1, RP11-986E7.7, RP4-555D20.4, RP4-60717.1, RP5-1043L13.1, RP5-1070A16.1, RP5-998N21.4, RPS6KA1, RSAD2, RUSC2, S100Z, SAMSN1, SASH3, SCG3, SCIMP, SCIN, SELE, SERPINA1, SERPINA3, SERPINA5, SERPINB1, SEZ6, SGMS2, SHANK3, SHH, SHISA6, SIGLEC1, SIGLEC10, SIGLEC11, SIGLEC17P, SIGLEC7, SIGLEC9, SIRPB2, SLA, SLAMF1, SLAMF6, SLAMF7, SLAMF8, SLC11A1, SLC16A5, SLC25A22, SLC2A5, SLC2A9, SLC46A2, SLC46A3, SLC7A7, SLC9A9, SLCO2B1, SLFN12L, SMPD3, SMPDL3A, SNAP25-AS1, SNX20, SP110, SP140, SPATA12, SPATC1, SPINT1, SPN, SPP1, SPTBN2, SQRDL, SRRM3, SSTR5, ST14, ST6GAL1, ST8SIA4, STAP1, STARD5, STAT5A, STEAP1B, STEAP4, STX11, STX1B, SULF1, SUSD1, SUSD4, SYK, SYT3, SYTL3, TACSTD2, TAGAP, TAGLN, TAGLN3, TBC1D2B, TBX19, TEC, TERC, TFEC, TGFBR2, TGM2, THBS2, THEMIS, THEMIS2, TIFAB, TLR1, TLR10, TLR2, TLR4, TLR5, TLR6, TLR7, TLR8, TMBIM1, TMC8, TMEM106A, TMEM110, TMEM140, TMEM150B, TMEM173, TMEM176B, TMEM86A, TNFRSF1B, TNFSF13, TNFSF13B, TNFSF15, TNFSF8, TPTE2P1, TRAPPC3L, TRIM22, TRIM38, TRNP1, TRPV4, TSLP, TTC7A, UCP2, USP43, VAMP3, VEGFC, VNN1, VNN2, VSIG4, VWA5A, WDFY4, XCR1, ZBED3-AS1, ZC3H12D, ZCCHC18, ZDHHC22, ZFP36, ZNF185, AATK, ABI3, AC008964.1, AC012593.1, AC109826.1, AIF1, ANKS1B, ANO4, AP001007.1, APOC2, APOL3, ARHGAP25, ARRB2, ASCL2, ASGR2, BATF, C16orf98, C19orf38, C1QA, C1orf162, CAPN9, CARD11, CARTPT, CCDC69, CCL5, CD2, CD27, CD33, CD37, CD3D, CD3E, CHI3L2, CLDND1, CLECL1, CMTM7, CNP, CPVL, CRYBB1, CSF2RA, CTA-384D8.34, CTC-378H22.1, CXCR3, DMBT1, DMRT2, EBI3, EDA, FA2H, FAM214B, FAM78A, FCGR1A, FCGR1B, FCGR1C, FERMT3, FOLH1, FOLR2, FTL, FTLP3, GBGT1, GIMAP1, GIMAP5, GNGT2, GPAT2, GPSM3, GYPC, GZMA, GZMH, GZMK, HAMP, HCN2, HCST, HLA-DPB1, HLA-DPB2, HOXD1, IER3, IL18, KIF19, KLRB1, LARP6, LGALS9, LILRA2, LINC00892, LINC00940, LY86, NANOS1, NCF1, NCF1B, NCF1C, NCF4, NINJ1, NIPA1, NKG7, OR52K3P, OTUD7A, PCED1B-AS1, PILRA, PLA2G2D, PRR15, PRR18, PTCRA, PTPLAD2, RAB40B, RASAL3, RETN, RGS10, RHOG, RNASET2, RNF130, RP11-134L10.1, RP11-214K3.24, RP11-244019.1, RP11-344B5.2, RP11-368J21.3, RP11-436F9.1, RP11-446E9.2, RP11-452C13.1, RP11-483P21.2, RP11-556E13.1, RP11-809C18.4, RP11-8L8.2, RP11-94L15.2, RP11-9G1.3, RP4-719C8.1, RP5-1091N2.9, RP5-899E9.1, RPGRIP1, SELPLG, SH2D1A, SIGLEC8, SIRPG, SIT1, SLC15A3, SOWAHD, SPI1, SULT1C2, SYNGR2, TBC1D2, TIMD4, TMEM119, TMEM52B, TMEM92, TNFAIP8L2, TPK1, TRAF3IP3, TREM2, TYROBP, UBASH3A, VAMP8, WAS, XCL2, and ZNF683.

In one embodiment, the expression score is determined from the expression status of a subset of genes. In other embodiments, the expression score is determined from the expression status of a subset of 10 genes. In other embodiments, the expression score is determined from the expression status of a subset of 100 genes. In other embodiments, the expression score is determined from the expression status of a subset of 500 genes. In other embodiments, the expression score is determined from the expression status of a subset of 1000 genes.

In another embodiment, the expression score is determined from the expression status of a subset of 5 or more genes. In some embodiments, the expression score is determined from the expression status of a subset of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 genes

As mentioned previously, the term expression refers to the process by which information from a gene is used in the synthesis of a functional gene product that enables it to produce a peptide/protein end product, and ultimately affect a phenotype, as the final effect. Similarly, “expression status” refers to identifying whether or not the functional gene or gene set is expressed and whether said expression is increased or decreased relative to a normal subject. An “expression score” further refers to a process of quantifying a functional gene or gene set expression level relative to a normal subject.

A progressive glioma is a tumor that occurs in the brain wherein the tumor growth worsens over time, thus resulting in decline of general overall health. Gliomas are classified into four grades (I, II, III, or IV), wherein the treatment and prognosis depend upon the glioma grade. In particular, grade IV gliomas are also referred to as high-grade gliomas. In some embodiments, the high-grade glioma is a glioblastoma. As used herein, a glioblastoma refers to an aggressive, fast growing type of glioma that occurs in the brain and spinal cord.

A glioblastoma also requires treatment to improve quality of health of the subject. In another embodiment, the subject identified with progressing glioblastoma is treated with surgery, chemotherapy, radiation, immunotherapy, gene therapy, or a combination thereof. In some embodiments, chemotherapy is given as a treatment for progressive glioblastoma. Such chemotherapy can be selected from the group consisting of alkylating agents, antimetabolites, anthracyclines, topoisomerase inhibitors, or corticosteroids. In another embodiment, the immunotherapy is given as a treatment for progressive glioblastoma. In other embodiments, the immunotherapy is an antibody therapy. In other embodiments, the immunotherapy is either a PD1 antibody therapy or a PD-L1 antibody therapy. In another embodiment, the gene therapy is given as a treatment for progressive glioblastoma. In another embodiment, the gene therapy includes but is not limited to gene editing (i.e., CRISPR-Cas gene editing systems), chimeric antigen receptor (CAR) gene therapy, and gene transfers (i.e., viral transfection and plasmid transformation).

For treating progressive glioma, a range of chemotherapy partners, including lomustine, carmustine, and temozolomide, can also be used in tandem with chemotherapy. Immunotherapy is also an option. Immune checkpoint inhibitors, predominantly targeting PD-1/PD-L1 and/or CTLA-4, can be used. Chimeric antigen receptor (CAR) T-cell therapy with genetically modified T cells can also be used Examples of treatment types can be found in Tan et al. (Tan, A C, Ashley, D M, López, G Y, Malinzak, M, Friedman, H S, Khasraw, M. Management of glioblastoma: State of the art and future directions. CA Cancer J Clin. 2020: 70:299-312), herein incorporated by reference in its entirety for its teaching concerning treatment of glioma.

In another embodiment, the subject identified with pseudo-progressing glioblastoma is treated with monitoring and/or steroids. In some embodiments, the subject identified with pseudo-progressing glioblastoma is treated with monitoring. In some embodiments, monitoring of the subject refers to the subject returning or continuing with regular or routine clinical treatments with a clinician or healthcare provider.

The clinical treatments can occur on a daily, weekly, monthly, or yearly basis. In some embodiments, the clinical treatments occur every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, or 365 days.

In other embodiments, the subject identified with pseudo-progressing glioblastoma is treated with steroids. In other embodiments, the steroid is selected from the group consisting of prednisolone, methylprednisolone, dexamethasone, hydrocortisone, or any combination thereof. For example, standard first-line chemotherapy can consist of temozolomide (75 mg/m² daily) during radiotherapy followed by a further six cycles of temozolomide (150-200 mg/m² on days 1-5 every 28 days). Lomustine, carmustine, and rechallenge with temozolomide are all potential options for treatment of progressive glioma as well. Further options include further surgical resection, reirradiation, systemic therapies such as lomustine or bevacizumab, combined approaches, or supportive care alone. Other options include radiotherapy, surgery, or palliative care.

In a specific example, single-agent bevacizumab can be used for the treatment of progressive glioma. Bevacizumab in combination with lomustine improves PFS compared with lomustine alone. Typically, the combination of bevacizumab with chemotherapy is recommended after failure on bevacizumab alone, which is different than the treatment for pseudoprogressive glioma. Importantly, those with progressive glioma should not be treated the same as those with pseudoprogressive glioma, particularly concerning bevacizumab. This is due to its effect on inflammatory and radio chemotherapy-induced changes in GBM vasculature. As discussed above, the term “pseudoprogression” is used to describe the phenomenon of subacute radio chemotherapy treatment-related sequelae in CNS tumors presenting as increasing lesion volume or new contrast enhancement on MRI suggestive of tumor progression. However, these patients often recover or stabilize spontaneously, usually without any change in treatment paradigm. (Brandsma D, Stalpers L, Taal W, Sminia P, van den Bent M J. Clinical features, mechanisms, and management of pseudoprogression in malignant gliomas. Lancet Oncol 2008; 9:453-461). Brandsma discloses that in patients managed with temozolomide chemoradiotherapy who have clinically asymptomatic progressive lesions at the end of treatment, adjuvant temozolomide should be continued; in clinically symptomatic patients, surgery should be considered. If mainly necrosis is noted during surgery, continuation of adjuvant temozolomide is logical. Trials on the treatment of recurrent malignant glioma should exclude patients with progression within the first 3 months after temozolomide chemoradiotherapy unless histological confirmation of tumor recurrence is available.

The etiology of pseudoprogression is thought to be due to vascular and oligodendroglia cell injury, leading to inflammation and increased BBB permeability. Because the enhancement seen in pseudoprogression can be mistaken for actual tumor progression, patients are often routed to bevacizumab as second-line therapy for recurrence. Bevacizumab has been observed to decrease the permeability of not only tumor-related leaky vasculature but also of radiation-induced leaky vasculature, thereby “curing” biopsy-proven pseudoprogression (Weinstein J S, Varallyay C G, Dosa E, et al. Superparamagnetic iron oxide nanoparticles: diagnostic magnetic resonance imaging and potential therapeutic applications in neurooncology and central nervous system inflammatory pathologies, a review. J Cereb Blood Flow Metab 2010; 30:15-35). This is particularly worrisome as pseudoprogression has been shown to be significantly associated with methylated MGMT promoter status and increased survival. The presence of a methylated MGMT promoter is associated with a significant survival benefit. The presence of a methylated MGMT promoter is associated with a significant survival benefit. The MGMT protein removes alkyl groups from the O⁶ position of guanine. Silencing of the MGMT promoter by methylation is clinically important because cancer cells produce less MGMT protein and cannot repair DNA alkylation by agents such as temozolomide.46 Bevacizumab may decrease the correlation between MGMT status and pseudoprogression (Thompson E M, Frenkel E P, Neuwelt E A. The paradoxical effect of bevacizumab in the therapy of malignant gliomas. Neurology. 2011 Jan. 4; 76 (1): 87-93).

In another embodiment, the biological sample is selected from the group consisting of blood, plasma, serum, cerebrospinal fluid, urine, saliva, stool, synovial fluid, a solid tissue sample, and a cell culture sample.

In one aspect, disclosed herein is a method for identifying a subject with progressing glioblastoma and further diagnosing them to confirm that the subject has progressing glioblastoma, the method comprising a) determining an expression status of one or more genes selected from 1000 genes or more isolated from a biological sample from the subject, b) using the expression status to determine an expression score relative to a normal subject, c) identifying a subject with an expression score greater than 25% as being at risk of having progressing glioblastoma relative to a normal subject, and subjecting the subject to a further diagnosis.

In some embodiments, an expression status of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, or more genes is selected from 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 1751, 1752, 1753, 1754, 1755, 1756, 1757, 1758, 1759, 1760, 1761, 1762, 1763, 1764, 1765, 1766, 1767, 1768, 1769, 1770, 1771, 1772, 1773, 1774, 1775, 1776, 1777, 1778, 1779, 1780, 1781, 1782, 1783, 1784, 1785, 1786, 1787, 1788, 1789, 1790, 1791, 1792, 1793, 1794, 1795, 1796, 1797, 1798, 1799, 1800, 1801, 1802, 1803, 1804, 1805, 1806, 1807, 1808, 1809, 1810, 1811, 1812, 1813, 1814, 1815, 1816, 1817, 1818, 1819, 1820, 1821, 1822, 1823, 1824, 1825, 1826, 1827, 1828, 1829, 1830, 1831, 1832, 1833, 1834, 1835, 1836, 1837, 1838, 1839, 1840, 1841, 1842, 1843, 1844, 1845, 1846, 1847, 1848, 1849, 1850, 1851, 1852, 1853, 1854, 1855, 1856, 1857, 1858, 1859, 1860, 1861, 1862, 1863, 1864, 1865, 1866, 1867, 1868, 1869, 1870, 1871, 1872, 1873, 1874, 1875, 1876, 1877, 1878, 1879, 1880, 1881, 1882, 1883, 1884, 1885, 1886, 1887, 1888, 1889, 1890, 1891, 1892, 1893, 1894, 1895, 1896, 1897, 1898, 1899, 1900, 1901, 1902, 1903, 1904, 1905, 1906, 1907, 1908, 1909, 1910, 1911, 1912, 1913, 1914, 1915, 1916, 1917, 1918, 1919, 1920, 1921, 1922, 1923, 1924, 1925, 1926, 1927, 1928, 1929, 1930, 1931, 1932, 1933, 1934, 1935, 1936, 1937, 1938, 1939, 1940, 1941, 1942, 1943, 1944, 1945, 1946, 1947, 1948, 1949, 1950, 1951, 1952, 1953, 1954, 1955, 1956, 1957, 1958, 1959, 1960, 1961, 1962, 1963, 1964, 1965, 1966, 1967, 1968, 1969, 1970, 1971, 1972, 1973, 1974, 1975, 1976, 1977, 1978, 1979, 1980, 1981, 1982, 1983, 1984, 1985, 1986, 1987, 1988, 1989, 1990, 1991, 1992, 1993, 1994, 1995, 1996, 1997, 1998, 1999, 2000, 2001, 2002, 2003, 2004, 2005, 2006, 2007, 2008, 2009, 2010, 2011, 2012, 2013, 2014, 2015, 2016, 2017, 2018, 2019, 2020, 2021, 2022, 2023, 2024, 2025, 2026, 2027, 2028, 2029, 2030, 2031, 2032, 2033, 2034, 2035, 2036, 2037, 2038, 2039, 2040, 2041, 2042, 2043, 2044, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2097, 2098, 2099, 2100, 2101, 2102, 2103, 2104, 2105, 2106, 2107, 2108, 2109, 2110, 2111, 2112, 2113, 2114, 2115, 2116, 2117, 2118, 2119, 2120, 2121, 2122, 2123, 2124, 2125, 2126, 2127, 2128, 2129, 2130, 2131, 2132, 2133, 2134, 2135, 2136, 2137, 2138, 2139, 2140, 2141, 2142, 2143, 2144, 2145, 2146, 2147, 2148, 2149, 2150, 2151, 2152, 2153, 2154, 2155, 2156, 2157, 2158, 2159, 2160, 2161, 2162, 2163, 2164, 2165, 2166, 2167, 2168, 2169, 2170, 2171, 2172, 2173, 2174, 2175, 2176, 2177, 2178, 2179, 2180, 2181, 2182, 2183, 2184, 2185, 2186, 2187, 2188, 2189, 2190, 2191, 2192, 2193, 2194, 2195, 2196, 2197, 2198, 2199, 2200, 2201, 2202, 2203, 2204, 2205, 2206, 2207, 2208, 2209, 2210, 2211, 2212, 2213, 2214, 2215, 2216, 2217, 2218, 2219, 2220, 2221, 2222, 2223, 2224, 2225, 2226, 2227, 2228, 2229, 2230, 2231, 2232, 2233, 2234, 2235, 2236, 2237, 2238, 2239, 2240, 2241, 2242, 2243, 2244, 2245, 2246, 2247, 2248, 2249, 2250, 2251, 2252, 2253, 2254, 2255, 2256, 2257, 2258, 2259, 2260, 2261, 2262, 2263, 2264, 2265, 2266, 2267, 2268, 2269, 2270, 2271, 2272, 2273, 2274, 2275, 2276, 2277, 2278, 2279, 2280, 2281, 2282, 2283, 2284, 2285, 2286, 2287, 2288, 2289, 2290, 2291, 2292, 2293, 2294, 2295, 2296, 2297, 2298, 2299, 2300, 2301, 2302, 2303, 2304, 2305, 2306, 2307, 2308, 2309, 2310, 2311, 2312, 2313, 2314, 2315, 2316, 2317, 2318, 2319, 2320, 2321, 2322, 2323, 2324, 2325, 2326, 2327, 2328, 2329, 2330, 2331, 2332, 2333, 2334, 2335, 2336, 2337, 2338, 2339, 2340, 2341, 2342, 2343, 2344, 2345, 2346, 2347, 2348, 2349, 2350, 2351, 2352, 2353, 2354, 2355, 2356, 2357, 2358, 2359, 2360, 2361, 2362, 2363, 2364, 2365, 2366, 2367, 2368, 2369, 2370, 2371, 2372, 2373, 2374, 2375, 2376, 2377, 2378, 2379, 2380, 2381, 2382, 2383, 2384, 2385, 2386, 2387, 2388, 2389, 2390, 2391, 2392, 2393, 2394, 2395, 2396, 2397, 2398, 2399, 2400, 2401, 2402, 2403, 2404, 2405, 2406, 2407, 2408, 2409, 2410, 2411, 2412, 2413, 2414, 2415, 2416, 2417, 2418, 2419, 2420, 2421, 2422, 2423, 2424, 2425, 2426, 2427, 2428, 2429, 2430, 2431, 2432, 2433, 2434, 2435, 2436, 2437, 2438, 2439, 2440, 2441, 2442, 2443, 2444, 2445, 2446, 2447, 2448, 2449, 2450, 2451, 2452, 2453, 2454, 2455, 2456, 2457, 2458, 2459, 2460, 2461, 2462, 2463, 2464, 2465, 2466, 2467, 2468, 2469, 2470, 2471, 2472, 2473, 2474, 2475, 2476, 2477, 2478, 2479, 2480, 2481, 2482, 2483, 2484, 2485, 2486, 2487, 2488, 2489, 2490, 2491, 2492, 2493, 2494, 2495, 2496, 2497, 2498, 2499, 2500, 2501, 2502, 2503, 2504, 2505, 2506, 2507, 2508, 2509, 2510, 2511, 2512, 2513, 2514, 2515, 2516, 2517, 2518, 2519, 2520, 2521, 2522, 2523, 2524, 2525, 2526, 2527, 2528, 2529, 2530, 2531, 2532, 2533, 2534, 2535, 2536, 2537, 2538, 2539, 2540, 2541, 2542, 2543, 2544, 2545, 2546, 2547, 2548, 2549, 2550, 2551, 2552, 2553, 2554, 2555, 2556, 2557, 2558, 2559, 2560, 2561, 2562, 2563, 2564, 2565, 2566, 2567, 2568, 2569, 2570, 2571, 2572, 2573, 2574, 2575, 2576, 2577, 2578, 2579, 2580, 2581, 2582, 2583, 2584, 2585, 2586, 2587, 2588, 2589, 2590, 2591, 2592, 2593, 2594, 2595, 2596, 2597, 2598, 2599, 2600, 2601, 2602, 2603, 2604, 2605, 2606, 2607, 2608, 2609, 2610, 2611, 2612, 2613, 2614, 2615, 2616, 2617, 2618, 2619, 2620, 2621, 2622, 2623, 2624, 2625, 2626, 2627, 2628, 2629, 2630, 2631, 2632, 2633, 2634, 2635, 2636, 2637, 2638, 2639, 2640, 2641, 2642, 2643, 2644, 2645, 2646, 2647, 2648, 2649, 2650, 2651, 2652, 2653, 2654, 2655, 2656, 2657, 2658, 2659, 2660, 2661, 2662, 2663, 2664, 2665, 2666, 2667, 2668, 2669, 2670, 2671, 2672, 2673, 2674, 2675, 2676, 2677, 2678, 2679, 2680, 2681, 2682, 2683, 2684, 2685, 2686, 2687, 2688, 2689, 2690, 2691, 2692, 2693, 2694, 2695, 2696, 2697, 2698, 2699, 2700, 2701, 2702, 2703, 2704, 2705, 2706, 2707, 2708, 2709, 2710, 2711, 2712, 2713, 2714, 2715, 2716, 2717, 2718, 2719, 2720, 2721, 2722, 2723, 2724, 2725, 2726, 2727, 2728, 2729, 2730, 2731, 2732, 2733, 2734, 2735, 2736, 2737, 2738, 2739, 2740, 2741, 2742, 2743, 2744, 2745, 2746, 2747, 2748, 2749, 2750, 2751, 2752, 2753, 2754, 2755, 2756, 2757, 2758, 2759, 2760, 2761, 2762, 2763, 2764, 2765, 2766, 2767, 2768, 2769, 2770, 2771, 2772, 2773, 2774, 2775, 2776, 2777, 2778, 2779, 2780, 2781, 2782, 2783, 2784, 2785, 2786, 2787, 2788, 2789, 2790, 2791, 2792, 2793, 2794, 2795, 2796, 2797, 2798, 2799, 2800, 2801, 2802, 2803, 2804, 2805, 2806, 2807, 2808, 2809, 2810, 2811, 2812, 2813, 2814, 2815, 2816, 2817, 2818, 2819, 2820, 2821, 2822, 2823, 2824, 2825, 2826, 2827, 2828, 2829, 2830, 2831, 2832, 2833, 2834, 2835, 2836, 2837, 2838, 2839, 2840, 2841, 2842, 2843, 2844, 2845, 2846, 2847, 2848, 2849, 2850, 2851, 2852, 2853, 2854, 2855, 2856, 2857, 2858, 2859, 2860, 2861, 2862, 2863, 2864, 2865, 2866, 2867, 2868, 2869, 2870, 2871, 2872, 2873, 2874, 2875, 2876, 2877, 2878, 2879, 2880, 2881, 2882, 2883, 2884, 2885, 2886, 2887, 2888, 2889, 2890, 2891, 2892, 2893, 2894, 2895, 2896, 2897, 2898, 2899, 2900, 2901, 2902, 2903, 2904, 2905, 2906, 2907, 2908, 2909, 2910, 2911, 2912, 2913, 2914, 2915, 2916, 2917, 2918, 2919, 2920, 2921, 2922, 2923, 2924, 2925, 2926, 2927, 2928, 2929, 2930, 2931, 2932, 2933, 2934, 2935, 2936, 2937, 2938, 2939, 2940, 2941, 2942, 2943, 2944, 2945, 2946, 2947, 2948, 2949, 2950, 2951, 2952, 2953, 2954, 2955, 2956, 2957, 2958, 2959, 2960, 2961, 2962, 2963, 2964, 2965, 2966, 2967, 2968, 2969, 2970, 2971, 2972, 2973, 2974, 2975, 2976, 2977, 2978, 2979, 2980, 2981, 2982, 2983, 2984, 2985, 2986, 2987, 2988, 2989, 2990, 2991, 2992, 2993, 2994, 2995, 2996, 2997, 2998, 2999, 3000, 3001, 3002, 3003, 3004, 3005, 3006, 3007, 3008, 3009, 3010, 3011, 3012, 3013, 3014, 3015, 3016, 3017, 3018, 3019, 3020, 3021, 3022, 3023, 3024, 3025, 3026, 3027, 3028, 3029, 3030, 3031, 3032, 3033, 3034, 3035, 3036, 3037, 3038, 3039, 3040, 3041, 3042, 3043, 3044, 3045, 3046, 3047, 3048, 3049, 3050, 3051, 3052, 3053, 3054, 3055, 3056, 3057, 3058, 3059, 3060, 3061, 3062, 3063, 3064, 3065, 3066, 3067, 3068, 3069, 3070, 3071, 3072, 3073, 3074, 3075, 3076, 3077, 3078, 3079, 3080, 3081, 3082, 3083, 3084, 3085, 3086, 3087, 3088, 3089, 3090, 3091, 3092, 3093, 3094, 3095, 3096, 3097, 3098, 3099, 3100, 3101, 3102, 3103, 3104, 3105, 3106, 3107, 3108, 3109, 3110, 3111, 3112, 3113, 3114, 3115, 3116, 3117, 3118, 3119, 3120, 3121, 3122, 3123, 3124, 3125, 3126, 3127, 3128, 3129, 3130, 3131, 3132, 3133, 3134, 3135, 3136, 3137, 3138, 3139, 3140, 3141, 3142, 3143, 3144, 3145, 3146, 3147, 3148, 3149, 3150, 3151, 3152, 3153, 3154, 3155, 3156, 3157, 3158, 3159, 3160, 3161, 3162, 3163, 3164, 3165, 3166, 3167, 3168, 3169, 3170, 3171, 3172, 3173, 3174, 3175, 3176, 3177, 3178, 3179, 3180, 3181, 3182, 3183, 3184, 3185, 3186, 3187, 3188, 3189, 3190, 3191, 3192, 3193, 3194, 3195, 3196, 3197, 3198, 3199, 3200, 3201, 3202, 3203, 3204, 3205, 3206, 3207, 3208, 3209, 3210, 3211, 3212, 3213, 3214, 3215, 3216, 3217, 3218, 3219, 3220, 3221, 3222, 3223, 3224, 3225, 3226, 3227, 3228, 3229, 3230, 3231, 3232, 3233, 3234, 3235, 3236, 3237, 3238, 3239, 3240, 3241, 3242, 3243, 3244, 3245, 3246, 3247, 3248, 3249, 3250, 3251, 3252, 3253, 3254, 3255, 3256, 3257, 3258, 3259, 3260, 3261, 3262, 3263, 3264, 3265, 3266, 3267, 3268, 3269, 3270, 3271, 3272, 3273, 3274, 3275, 3276, 3277, 3278, 3279, 3280, 3281, 3282, 3283, 3284, 3285, 3286, 3287, 3288, 3289, 3290, 3291, 3292, 3293, 3294, 3295, 3296, 3297, 3298, 3299, 3300, 3301, 3302, 3303, 3304, 3305, 3306, 3307, 3308, 3309, 3310, 3311, 3312, 3313, 3314, 3315, 3316, 3317, 3318, 3319, 3320, 3321, 3322, 3323, 3324, 3325, 3326, 3327, 3328, 3329, 3330, 3331, 3332, 3333, 3334, 3335, 3336, 3337, 3338, 3339, 3340, 3341, 3342, 3343, 3344, 3345, 3346, 3347, 3348, 3349, 3350, 3351, 3352, 3353, 3354, 3355, 3356, 3357, 3358, 3359, 3360, 3361, 3362, 3363, 3364, 3365, 3366, 3367, 3368, 3369, 3370, 3371, 3372, 3373, 3374, 3375, 3376, 3377, 3378, 3379, 3380, 3381, 3382, 3383, 3384, 3385, 3386, 3387, 3388, 3389, 3390, 3391, 3392, 3393, 3394, 3395, 3396, 3397, 3398, 3399, 3400, 3401, 3402, 3403, 3404, 3405, 3406, 3407, 3408, 3409, 3410, 3411, 3412, 3413, 3414, 3415, 3416, 3417, 3418, 3419, 3420, 3421, 3422, 3423, 3424, 3425, 3426, 3427, 3428, 3429, 3430, 3431, 3432, 3433, 3434, 3435, 3436, 3437, 3438, 3439, 3440, 3441, 3442, 3443, 3444, 3445, 3446, 3447, 3448, 3449, 3450, 3451, 3452, 3453, 3454, 3455, 3456, 3457, 3458, 3459, 3460, 3461, 3462, 3463, 3464, 3465, 3466, 3467, 3468, 3469, 3470, 3471, 3472, 3473, 3474, 3475, 3476, 3477, 3478, 3479, 3480, 3481, 3482, 3483, 3484, 3485, 3486, 3487, 3488, 3489, 3490, 3491, 3492, 3493, 3494, 3495, 3496, 3497, 3498, 3499, 3500, 3501, 3502, 3503, 3504, 3505, 3506, 3507, 3508, 3509, 3510, 3511, 3512, 3513, 3514, 3515, 3516, 3517, 3518, 3519, 3520, 3521, 3522, 3523, 3524, 3525, 3526, 3527, 3528, 3529, 3530, 3531, 3532, 3533, 3534, 3535, 3536, 3537, 3538, 3539, 3540, 3541, 3542, 3543, 3544, 3545, 3546, 3547, 3548, 3549, 3550, 3551, 3552, 3553, 3554, 3555, 3556, 3557, 3558, 3559, 3560, 3561, 3562, 3563, 3564, 3565, 3566, 3567, 3568, 3569, 3570, 3571, 3572, 3573, 3574, 3575, 3576, 3577, 3578, 3579, 3580, 3581, 3582, 3583, 3584, 3585, 3586, 3587, 3588, 3589, 3590, 3591, 3592, 3593, 3594, 3595, 3596, 3597, 3598, 3599, 3600, 3601, 3602, 3603, 3604, 3605, 3606, 3607, 3608, 3609, 3610, 3611, 3612, 3613, 3614, 3615, 3616, 3617, 3618, 3619, 3620, 3621, 3622, 3623, 3624, 3625, 3626, 3627, 3628, 3629, 3630, 3631, 3632, 3633, 3634, 3635, 3636, 3637, 3638, 3639, 3640, 3641, 3642, 3643, 3644, 3645, 3646, 3647, 3648, 3649, 3650, 3651, 3652, 3653, 3654, 3655, 3656, 3657, 3658, 3659, 3660, 3661, 3662, 3663, 3664, 3665, 3666, 3667, 3668, 3669, 3670, 3671, 3672, 3673, 3674, 3675, 3676, 3677, 3678, 3679, 3680, 3681, 3682, 3683, 3684, 3685, 3686, 3687, 3688, 3689, 3690, 3691, 3692, 3693, 3694, 3695, 3696, 3697, 3698, 3699, 3700, 3701, 3702, 3703, 3704, 3705, 3706, 3707, 3708, 3709, 3710, 3711, 3712, 3713, 3714, 3715, 3716, 3717, 3718, 3719, 3720, 3721, 3722, 3723, 3724, 3725, 3726, 3727, 3728, 3729, 3730, 3731, 3732, 3733, 3734, 3735, 3736, 3737, 3738, 3739, 3740, 3741, 3742, 3743, 3744, 3745, 3746, 3747, 3748, 3749, 3750, 3751, 3752, 3753, 3754, 3755, 3756, 3757, 3758, 3759, 3760, 3761, 3762, 3763, 3764, 3765, 3766, 3767, 3768, 3769, 3770, 3771, 3772, 3773, 3774, 3775, 3776, 3777, 3778, 3779, 3780, 3781, 3782, 3783, 3784, 3785, 3786, 3787, 3788, 3789, 3790, 3791, 3792, 3793, 3794, 3795, 3796, 3797, 3798, 3799, 3800, 3801, 3802, 3803, 3804, 3805, 3806, 3807, 3808, 3809, 3810, 3811, 3812, 3813, 3814, 3815, 3816, 3817, 3818, 3819, 3820, 3821, 3822, 3823, 3824, 3825, 3826, 3827, 3828, 3829, 3830, 3831, 3832, 3833, 3834, 3835, 3836, 3837, 3838, 3839, 3840, 3841, 3842, 3843, 3844, 3845, 3846, 3847, 3848, 3849, 3850, 3851, 3852, 3853, 3854, 3855, 3856, 3857, 3858, 3859, 3860, 3861, 3862, 3863, 3864, 3865, 3866, 3867, 3868, 3869, 3870, 3871, 3872, 3873, 3874, 3875, 3876, 3877, 3878, 3879, 3880, 3881, 3882, 3883, 3884, 3885, 3886, 3887, 3888, 3889, 3890, 3891, 3892, 3893, 3894, 3895, 3896, 3897, 3898, 3899, 3900, 3901, 3902, 3903, 3904, 3905, 3906, 3907, 3908, 3909, 3910, 3911, 3912, 3913, 3914, 3915, 3916, 3917, 3918, 3919, 3920, 3921, 3922, 3923, 3924, 3925, 3926, 3927, 3928, 3929, 3930, 3931, 3932, 3933, 3934, 3935, 3936, 3937, 3938, 3939, 3940, 3941, 3942, 3943, 3944, 3945, 3946, 3947, 3948, 3949, 3950, 3951, 3952, 3953, 3954, 3955, 3956, 3957, 3958, 3959, 3960, 3961, 3962, 3963, 3964, 3965, 3966, 3967, 3968, 3969, 3970, 3971, 3972, 3973, 3974, 3975, 3976, 3977, 3978, 3979, 3980, 3981, 3982, 3983, 3984, 3985, 3986, 3987, 3988, 3989, 3990, 3991, 3992, 3993, 3994, 3995, 3996, 3997, 3998, 3999, 4000, 4001, 4002, 4003, 4004, 4005, 4006, 4007, 4008, 4009, 4010, 4011, 4012, 4013, 4014, 4015, 4016, 4017, 4018, 4019, 4020, 4021, 4022, 4023, 4024, 4025, 4026, 4027, 4028, 4029, 4030, 4031, 4032, 4033, 4034, 4035, 4036, 4037, 4038, 4039, 4040, 4041, 4042, 4043, 4044, 4045, 4046, 4047, 4048, 4049, 4050, 4051, 4052, 4053, 4054, 4055, 4056, 4057, 4058, 4059, 4060, 4061, 4062, 4063, 4064, 4065, 4066, 4067, 4068, 4069, 4070, 4071, 4072, 4073, 4074, 4075, 4076, 4077, 4078, 4079, 4080, 4081, 4082, 4083, 4084, 4085, 4086, 4087, 4088, 4089, 4090, 4091, 4092, 4093, 4094, 4095, 4096, 4097, 4098, 4099, 4100, 4101, 4102, 4103, 4104, 4105, 4106, 4107, 4108, 4109, 4110, 4111, 4112, 4113, 4114, 4115, 4116, 4117, 4118, 4119, 4120, 4121, 4122, 4123, 4124, 4125, 4126, 4127, 4128, 4129, 4130, 4131, 4132, 4133, 4134, 4135, 4136, 4137, 4138, 4139, 4140, 4141, 4142, 4143, 4144, 4145, 4146, 4147, 4148, 4149, 4150, 4151, 4152, 4153, 4154, 4155, 4156, 4157, 4158, 4159, 4160, 4161, 4162, 4163, 4164, 4165, 4166, 4167, 4168, 4169, 4170, 4171, 4172, 4173, 4174, 4175, 4176, 4177, 4178, 4179, 4180, 4181, 4182, 4183, 4184, 4185, 4186, 4187, 4188, 4189, 4190, 4191, 4192, 4193, 4194, 4195, 4196, 4197, 4198, 4199, 4200, 4201, 4202, 4203, 4204, 4205, 4206, 4207, 4208, 4209, 4210, 4211, 4212, 4213, 4214, 4215, 4216, 4217, 4218, 4219, 4220, 4221, 4222, 4223, 4224, 4225, 4226, 4227, 4228, 4229, 4230, 4231, 4232, 4233, 4234, 4235, 4236, 4237, 4238, 4239, 4240, 4241, 4242, 4243, 4244, 4245, 4246, 4247, 4248, 4249, 4250, 4251, 4252, 4253, 4254, 4255, 4256, 4257, 4258, 4259, 4260, 4261, 4262, 4263, 4264, 4265, 4266, 4267, 4268, 4269, 4270, 4271, 4272, 4273, 4274, 4275, 4276, 4277, 4278, 4279, 4280, 4281, 4282, 4283, 4284, 4285, 4286, 4287, 4288, 4289, 4290, 4291, 4292, 4293, 4294, 4295, 4296, 4297, 4298, 4299, 4300, 4301, 4302, 4303, 4304, 4305, 4306, 4307, 4308, 4309, 4310, 4311, 4312, 4313, 4314, 4315, 4316, 4317, 4318, 4319, 4320, 4321, 4322, 4323, 4324, 4325, 4326, 4327, 4328, 4329, 4330, 4331, 4332, 4333, 4334, 4335, 4336, 4337, 4338, 4339, 4340, 4341, 4342, 4343, 4344, 4345, 4346, 4347, 4348, 4349, 4350, 4351, 4352, 4353, 4354, 4355, 4356, 4357, 4358, 4359, 4360, 4361, 4362, 4363, 4364, 4365, 4366, 4367, 4368, 4369, 4370, 4371, 4372, 4373, 4374, 4375, 4376, 4377, 4378, 4379, 4380, 4381, 4382, 4383, 4384, 4385, 4386, 4387, 4388, 4389, 4390, 4391, 4392, 4393, 4394, 4395, 4396, 4397, 4398, 4399, 4400, 4401, 4402, 4403, 4404, 4405, 4406, 4407, 4408, 4409, 4410, 4411, 4412, 4413, 4414, 4415, 4416, 4417, 4418, 4419, 4420, 4421, 4422, 4423, 4424, 4425, 4426, 4427, 4428, 4429, 4430, 4431, 4432, 4433, 4434, 4435, 4436, 4437, 4438, 4439, 4440, 4441, 4442, 4443, 4444, 4445, 4446, 4447, 4448, 4449, 4450, 4451, 4452, 4453, 4454, 4455, 4456, 4457, 4458, 4459, 4460, 4461, 4462, 4463, 4464, 4465, 4466, 4467, 4468, 4469, 4470, 4471, 4472, 4473, 4474, 4475, 4476, 4477, 4478, 4479, 4480, 4481, 4482, 4483, 4484, 4485, 4486, 4487, 4488, 4489, 4490, 4491, 4492, 4493, 4494, 4495, 4496, 4497, 4498, 4499, 4500, 4501, 4502, 4503, 4504, 4505, 4506, 4507, 4508, 4509, 4510, 4511, 4512, 4513, 4514, 4515, 4516, 4517, 4518, 4519, 4520, 4521, 4522, 4523, 4524, 4525, 4526, 4527, 4528, 4529, 4530, 4531, 4532, 4533, 4534, 4535, 4536, 4537, 4538, 4539, 4540, 4541, 4542, 4543, 4544, 4545, 4546, 4547, 4548, 4549, 4550, 4551, 4552, 4553, 4554, 4555, 4556, 4557, 4558, 4559, 4560, 4561, 4562, 4563, 4564, 4565, 4566, 4567, 4568, 4569, 4570, 4571, 4572, 4573, 4574, 4575, 4576, 4577, 4578, 4579, 4580, 4581, 4582, 4583, 4584, 4585, 4586, 4587, 4588, 4589, 4590, 4591, 4592, 4593, 4594, 4595, 4596, 4597, 4598, 4599, 4600, 4601, 4602, 4603, 4604, 4605, 4606, 4607, 4608, 4609, 4610, 4611, 4612, 4613, 4614, 4615, 4616, 4617, 4618, 4619, 4620, 4621, 4622, 4623, 4624, 4625, 4626, 4627, 4628, 4629, 4630, 4631, 4632, 4633, 4634, 4635, 4636, 4637, 4638, 4639, 4640, 4641, 4642, 4643, 4644, 4645, 4646, 4647, 4648, 4649, 4650, 4651, 4652, 4653, 4654, 4655, 4656, 4657, 4658, 4659, 4660, 4661, 4662, 4663, 4664, 4665, 4666, 4667, 4668, 4669, 4670, 4671, 4672, 4673, 4674, 4675, 4676, 4677, 4678, 4679, 4680, 4681, 4682, 4683, 4684, 4685, 4686, 4687, 4688, 4689, 4690, 4691, 4692, 4693, 4694, 4695, 4696, 4697, 4698, 4699, 4700, 4701, 4702, 4703, 4704, 4705, 4706, 4707, 4708, 4709, 4710, 4711, 4712, 4713, 4714, 4715, 4716, 4717, 4718, 4719, 4720, 4721, 4722, 4723, 4724, 4725, 4726, 4727, 4728, 4729, 4730, 4731, 4732, 4733, 4734, 4735, 4736, 4737, 4738, 4739, 4740, 4741, 4742, 4743, 4744, 4745, 4746, 4747, 4748, 4749, 4750, 4751, 4752, 4753, 4754, 4755, 4756, 4757, 4758, 4759, 4760, 4761, 4762, 4763, 4764, 4765, 4766, 4767, 4768, 4769, 4770, 4771, 4772, 4773, 4774, 4775, 4776, 4777, 4778, 4779, 4780, 4781, 4782, 4783, 4784, 4785, 4786, 4787, 4788, 4789, 4790, 4791, 4792, 4793, 4794, 4795, 4796, 4797, 4798, 4799, 4800, 4801, 4802, 4803, 4804, 4805, 4806, 4807, 4808, 4809, 4810, 4811, 4812, 4813, 4814, 4815, 4816, 4817, 4818, 4819, 4820, 4821, 4822, 4823, 4824, 4825, 4826, 4827, 4828, 4829, 4830, 4831, 4832, 4833, 4834, 4835, 4836, 4837, 4838, 4839, 4840, 4841, 4842, 4843, 4844, 4845, 4846, 4847, 4848, 4849, 4850, 4851, 4852, 4853, 4854, 4855, 4856, 4857, 4858, 4859, 4860, 4861, 4862, 4863, 4864, 4865, 4866, 4867, 4868, 4869, 4870, 4871, 4872, 4873, 4874, 4875, 4876, 4877, 4878, 4879, 4880, 4881, 4882, 4883, 4884, 4885, 4886, 4887, 4888, 4889, 4890, 4891, 4892, 4893, 4894, 4895, 4896, 4897, 4898, 4899, 4900, 4901, 4902, 4903, 4904, 4905, 4906, 4907, 4908, 4909, 4910, 4911, 4912, 4913, 4914, 4915, 4916, 4917, 4918, 4919, 4920, 4921, 4922, 4923, 4924, 4925, 4926, 4927, 4928, 4929, 4930, 4931, 4932, 4933, 4934, 4935, 4936, 4937, 4938, 4939, 4940, 4941, 4942, 4943, 4944, 4945, 4946, 4947, 4948, 4949, 4950, 4951, 4952, 4953, 4954, 4955, 4956, 4957, 4958, 4959, 4960, 4961, 4962, 4963, 4964, 4965, 4966, 4967, 4968, 4969, 4970, 4971, 4972, 4973, 4974, 4975, 4976, 4977, 4978, 4979, 4980, 4981, 4982, 4983, 4984, 4985, 4986, 4987, 4988, 4989, 4990, 4991, 4992, 4993, 4994, 4995, 4996, 4997, 4998, 4999, 5000, or more genes isolated from a biological sample. In another embodiment, the subject is identified with an expression score of 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as being at risk of having progressing glioblastoma relative to a normal subject.

In another embodiment, the expression status is determined from any one gene including but not limited to AC005592.2, AC007879.7, ACTA2, ACTG2, ADAM8, ADM, ANGPTL4, ANKRD1, ANPEP, AOC3, APCDD1L, ATF5, ATP2A3, ATP6VOD2, B3GNT7, BCL3, C15orf48, CA9, CCIN, CCR3, CCR7, CD1C, CD300E, CLCF1, CLDN23, CNN2, COL1A1, COL1A2, COL3A1, COL5A1, COL6A3, CSTA, CXCR4, CYR61, DMBX1, DPP4, EGLN3, EMILIN2, FAM46B, FN1, FOSL1, FOSL2, FPR2, GALNT5, GPR18, HBEGF, HOXB6, HOXB7, ICAM1, IL1R1, IL7R, IL8, IRF1, ITK, KLF4, LCK, LIF, LOX, LUCAT1, MAFF, MAPK13, MEDAG, MEFV, MMP11, MMP9, NABP1, NAMPT, NDRG1, NFKB1A, NLRP12, P4HA3, PAX8, PDLIM1, PIM1, PLAUR, PLEKHS1, PPP1R15A, PTGER2, RAB43, RAC2, RGS1, RGS2, RHOH, RND3, ROR2, RP11-1149023.2, RP11-1149023.3, RP11-290F20.3, RP11-59D5_B.2, RP5-1021120.2, S100A8, S100A9, SDC1, SERPINE1, SLC16A3, SNAI1, SPOCD1, SUSD2, THBD, THBS1, TMEM217, TNFAIP6, TNFAIP8, TNFRSF10D, TREM1, TREML2, TREML3P, VDR, C3H12A, ABCA6, AC002059.10, AC005932.1, AC008060.7, AC011558.5, AC017076.5, AC098823.3, ACSM5, ACTA2-AS1, ADRA2B, APLNR, ARHGEF5, ARRDC2, BEST1, BHLHE41, CD55, CD83, CDK18, CLCA2, CLMN, COL4A5, CPXM2, CTC-1337H24.1, CTD-2034121.2, CXorf65, CYB5R2, DNM1P47, DYSF, EHF, ELOVL1, EVI2A, FAM124A, FAM53B, FGF20, FKBP11, FMN1, GALNT6, GPR37, GTSF1, HMSD, HOXD-AS1, HPN-AS1, ICOSLG, IL9R, IP6K3, KANK4, KCNJ2-AS1, KCNN4, KEL, LAG3, LHB, LINC01010, LRMP, LRP2, LRRC63, LURAP1, MFSD6L, MGST1, MIR24-2, MS4A4E, NFKBID, NME8, OSTF1, P2RX1, PATL2, PCP2, PCSK6, PDLIM7, PLA2G7, PLD1, PM20D1, PPM1N, PRG4, RP11-1094M14.8, RP11-136K7.2, RP11-161H23.5, RP11-16K12.1, RP11-350G8.5, RP11-399019.9, RP11-434H6.6, RP11-597D13.7. RP11-700H6.1, RP11-703116.1, RP11-883G14.1, RP3-47704.14, RP5-943J3.1, RP6-99M1.2, RPS10P7, SAMD3, SEPP1, SH2D3A, SLC45A3, SLC52A1, ST18, SVIP, TM6SF1, TMC7, TMEM139, TMPRSS13, TPM2, TRIM59, TRPC2, TRPM6, TRPV5, U91328.20, UCN2, ABCG1, ABI3BP, AC003003.5, AC004988.1, AC005082.12, AC006129.1, AC006129.2, AC011899.9, AC026202.3, ACAT2, ACP5, ACSL1, ADAM28, ADAMTS1, ADAMTS12, ADAP2, ADRB2, AF127936.5, AKR1B10, AKR1B15, ALOX12B, ALOX5, ALOX5AP, AMICA1, AMPD3, ANKRD22, AOAH, AP000476.1, AP000783.1, APBB1IP, APH1B, APOBEC3C, APOBR, ARHGAP30, ARHGDIB, ARHGEF34P, ARHGEF35, ARL11, ARL3, ARRDC4, ASB12, ATP8B4, AXL, B4GALT1, BCL2A1, BCR, BIN2, BIRC3, BMP2K, BTK, C10orf10, C11orf21, C11orf45, C16orf54, C16orf92, C19orf35, C1QC, C2, C3AR1, C4BPB, C5AR1, C5AR2, C9orf47, C9orf66, CABP7, CACNA2D4, CASP1, CASP10, CASP5, CASS4, CBFA2T3, CCL18, CCL2, CCL20, CCL22, CCL8, CCR1, CCR2, CCR4, CCR5, CCRL2, CD14, CD163, CD180, CD1D, CD200R1, CD274, CD28, CD300A, CD300C, CD300LB, CD300LF, CD3G, CD4, CD40, CD40LG, CD53, CD59, CD68, CD69, CD80, CD84, CD86, CD96, CDCP1, CDKN1A, CEACAM21, CEACAM4, CEBPA, CECR1, CELSR3, CFH, CFLAR, CHCHD7, CHRM3-AS2, CIITA, CLEC10A, CLEC12A, CLEC12B, CLEC2B, CLEC7A, CLSTN3, CMAHP, CMKLR1, CMPK2, CNN1, COMP, COQ3, CP, CPA6, CPM, CR1, CREG1, CRTAM, CSF1R, CSF2RB, CSF3R, CTC-205M6.5, CTC-250114.6, CTD-2258A20.5, CTD-2306M10.1, CTD-2540B15.7, CTD-2587H19.1, CTSB, CTSC, CTSH, CTSL, CTSS, CTSZ, CXCL12, CXCL16, CXCL6, CXCR2P1, CXCR6, CXorf21, CYBB, CYP19A1, CYTH4, CYTIP, DAB2, DAPP1, DCN, DCSTAMP, DDO, DDX58, DEC1, DENND3, DLGAP1-AS3, DLX2, DLX6, DLX6-AS1, DMRTC1B, DNAJC5B, DNASE2B, DOCK2, DOCK8, DOK3, DPY19L2P4, DRAM1, DSC2, DUSP1, EFCAB4B, ELL2, EMB, EMR1, EMR2, EMR4P, EPSTI1, EVI2B, F13A1, FAM105A FAM177B, FAM20A, FAM213A, FAM57B, FASLG, FAXC, FBP1, FBXO32, FCER1A, FCER1G, FCGBP, FCGR2A, FCGR2B, FCGR3A, FCRL6, FFAR4, FGD2, FGL2, FGR, FH, FLVCR2, FMO2, FMO4, FMOD, FOXD4, FPR1, FPR3, FSD1, FUCA1, FUT9, GAB3, GADD45B, GAL3ST4, GALNT12, GAPT, GATA3, GBP1P1, GBP2, GBP3, GCH1, GCNT1, GDAP1L1, GDI1, GIMAP2, GIMAP4, GIMAP6, GIMAP7, GIMAP5, GLIPR1, GLRX, GM2A, GNA15, GPNMB, GPR132, GPR141, GPR160, GPR183, GPR34, GPR65, GPR82, GPR84, GPR85, GPRIN3, GRM8, GSK3A, GVINP1, GVQW1, HAVCR2, HCK, HCLS1, HCN4, HLA-DMA, HLA-DMB, HLA-DOA, HLA-DPA1, HLA-DQA2, HLA-DRA, HNMT, HPGDS, HPSE, HTR2B, HTRA4, IFI30, IFI44L, IFIH1, IFIT3, IGF1, IGSF6, IKBKE, IKZF1, IL10, IL10RA, IL12RB1, IL15RA, IL16, IL17RA, IL18BP, IL1A, IL1B, IL1RN, IL21R, IL2RA, IL2RB, IL2RG, IL4I1, IL4R, IL6, ILDR1, IRAK3, IRF4, IRF5, IRF8, IRF9, ITGAL, ITGAM, ITGAX, ITGB2, JPH2, KCNF1, KCNJ5, KCNK13, KCNMB1, KCNQ1, KLC2, KLHDC7B, KLHL6, KLRC1, KLRD1, KMO, KYNU, LA16c-380H5.5, LACTB, LAIR1, LAMP1, LANCL2, LAPTMS, LAT2, LAYN, LBP, LCP1, LCP2, LGALS17A, LILRA1, LILRA6, LILRB1, LILRB2, LILRB4, LILRB5, LINC00539, LINC00578, LINC00702, LINC00968, LPAR5, LPCAT2, LPXN, LRRC25, LRRC39, LRRTM1, LTBP2, LTBR, LUM, LURAP1L, LY9, LYN, LYSMD2, LYVE1, LYZ, M6PR, MAF, MAFB, MALL, MAN1A1, MAN1C1, MAOB, MAP3KS, MCOLN2, MERTK, MGAT4A, MGAT5B, MITF, MKNK1, MLKL, MLPH, MMD2, MNDA, MPEG1, MRC1, MRC1L1, MS4A4A, MS4A6A, MS4A7, MSR1, MX1, MX2, MYO1F, NAAA, NCF2, NCKAP1L, NDUFA6-AS1, NFAM1, NFATC2, NGFR, NGFRAP1, NLRC4, NPL, NRROS, NUDT16P, OAS1, OAS2, OLFML2B, OLFML3, OLR1, OSM, OTUD1, P2RY13, P2RY6, P2RY8, PALD1, PALMD, PAPPA, PARP14, PARP15, PCED1B, PDCD1LG2, PDZD4, PGA5, PIK3AP1, PIK3CD, PIK3R5, PIRT, PJA1, PLA2G15, PLB1, PLBD1, PLCG2, PLEK, PLEKHA3P1, PLEKHO2, PLXDC2, POU2F2, PPARG, PPP1R14C, PPP1R17, PRDM12, PRKCD, PRLR, PRRT4, PTAFR, PTCHD2, PTGDR, PTGER4, PTGFR, PTPN22, PTPN7, PTPRC, PTX3, PYHIN1, RAB20, RAB38, RAB44, RAD51B, RARRES1, RASGRP4, RASL11B, RBM47, RERGL, RGS18, RHBDF2, RHOBTB2, RIN3, RIPK3, RNASE2, RNASE3, RNF112, RNF144B, RP11-1024P17.1, RP11-10K16.1, RP11-1149M10.2, RP11-117D22.2, RP11-119F19.2, RP11-121A8.1, RP11-153M7.1, RP11-153M7.3, RP11-196G18.3, RP11-23P13.6, RP11-245J24.1, RP11-271K11.5, RP11-307C19.1, RP11-325F22.2, RP11-346C20.4, RP11-353N4.5, RP11-357K6.1, RP11-363E6.3, RP11-364B14.3, RP11-379B8.1, RP11-384L8.1, RP11-3B12.1, RP11-403P17.5, RP11-439A17.9, RP11-477J21.6, RP11-47122.3, RP11-497H16.7, RP11-523018.5, RP11-565J7.1, RP11-588H23.3, RP11-626G11.4, RP11-676J15.1, RP11-70C1.1, RP11-733018.1, RP11-750H9.5, RP11-760N9.1, RP11-792D21.2, RP11-838N2.4, RP11-84C10.2, RP11-861A13.4, RP11-90P13.1, RP11-93B14.5, RP11-960L18.1, RP11-986E7.7, RP4-555D20.4, RP4-60717.1, RP5-1043L13.1, RP5-1070A16.1, RP5-998N21.4, RPS6KA1, RSAD2, RUSC2, S100Z, SAMSN1, SASH3, SCG3, SCIMP, SCIN, SELE, SERPINA1, SERPINA3, SERPINA5, SERPINB1, SEZ6, SGMS2, SHANK3, SHH, SHISA6, SIGLEC1, SIGLEC10, SIGLEC11, SIGLEC17P, SIGLEC7, SIGLEC9, SIRPB2, SLA, SLAMF1, SLAMF6, SLAMF7, SLAMF8, SLC11A1, SLC16A5, SLC25A22, SLC2AS, SLC2A9, SLC46A2, SLC46A3, SLC7A7, SLC9A9, SLCO2B1, SLFN12L, SMPD3, SMPDL3A, SNAP25-AS1, SNX20, SP110, SP140, SPATA12, SPATC1, SPINT1, SPN, SPP1, SPTBN2, SQRDL, SRRM3, SSTR5, ST14, ST6GAL1, ST8SIA4, STAP1, STARD5, STAT5A, STEAP1B, STEAP4, STX11, STX1B, SULF1, SUSD1, SUSD4, SYK, SYT3, SYTL3, TACSTD2, TAGAP, TAGLN, TAGLN3, TBC1D2B, TBX19, TEC, TERC, TFEC, TGFBR2, TGM2, THBS2, THEMIS, THEMIS2, TIFAB, TLR1, TLR10, TLR2, TLR4, TLR5, TLR6, TLR7, TLR8, TMBIM1, TMC8, TMEM106A, TMEM110, TMEM140, TMEM150B, TMEM173, TMEM176B, TMEM86A, TNFRSF1B, TNFSF13, TNFSF13B, TNFSF15, TNFSF8, TPTE2P1, TRAPPC3L, TRIM22, TRIM38, TRNP1, TRPV4, TSLP, TTC7A, UCP2, USP43, VAMP3, VEGFC, VNN1, VNN2, VSIG4, VWA5A, WDFY4, XCR1, ZBED3-AS1, ZC3H12D, ZCCHC18, ZDHHC22, ZFP36, ZNF185, AATK, ABI3, AC008964.1, AC012593.1, AC109826.1, AIF1, ANKS1B, ANO4, AP001007.1, APOC2, APOL3, ARHGAP25, ARRB2, ASCL2, ASGR2, BATF, C16orf98, C19orf38, C1QA, C1orf162, CAPN9, CARD11, CARTPT, CCDC69, CCL5, CD2, CD27, CD33, CD37, CD3D, CD3E, CHI3L2, CLDND1, CLECL1, CMTM7, CNP, CPVL, CRYBB1, CSF2RA, CTA-384D8.34, CTC-378H22.1, CXCR3, DMBT1, DMRT2, EBI3, EDA, FA2H, FAM214B, FAM78A, FCGR1A, FCGR1B, FCGR1C, FERMT3, FOLH1, FOLR2, FTL, FTLP3, GBGT1, GIMAP1, GIMAP5, GNGT2, GPAT2, GPSM3, GYPC, GZMA, GZMH, GZMK, HAMP, HCN2, HCST, HLA-DPB1, HLA-DPB2, HOXD1, IER3, IL18, KIF19, KLRB1, LARP6, LGALS9, LILRA2, LINC00892, LINC00940, LY86, NANOS1, NCF1, NCF1B, NCF1C, NCF4, NINJ1, NIPA1, NKG7, OR52K3P, OTUD7A, PCED1B-AS1, PILRA, PLA2G2D, PRR15, PRR18, PTCRA, PTPLAD2, RAB40B, RASAL3, RETN, RGS10, RHOG, RNASET2, RNF130, RP11-134L10.1, RP11-214K3.24, RP11-244019.1, RP11-344B5.2, RP11-368J21.3, RP11-436F9.1, RP11-446E9.2, RP11-452C13.1, RP11-483P21.2, RP11-556E13.1, RP11-809C18.4, RP11-8L8.2, RP11-94L15.2, RP11-9G1.3, RP4-719C8.1, RP5-1091N2.9, RP5-899E9.1, RPGRIP1, SELPLG, SH2D1A, SIGLEC8, SIRPG, SIT1, SLC15A3, SOWAHD, SPI1, SULT1C2, SYNGR2, TBC1D2, TIMD4, TMEM119, TMEM52B, TMEM92, TNFAIP8L2, TPK1, TRAF3IP3, TREM2, TYROBP, UBASH3A, VAMP8, WAS, XCL2, and ZNF683.

In one embodiment, the expression score is determined from the expression status of a subset of genes. In another embodiment, the expression score is determined from the expression status of a subset of 10 genes. In another embodiment, the expression score is determined from the expression status of a subset of 100 genes. In another embodiment, the expression score is determined from the expression status of a subset of 500 genes. In another embodiment, the expression score is determined from the expression status of a subset of 1000 genes.

In another embodiment, the expression score is determined from the expression status of a subset of 5 or more genes. In some embodiments, the expression score is determined from the expression status of a subset of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 genes.

Addition diagnostic methods can be performed to confirm the progressing glioblastoma. In another embodiment, said further diagnosis comprises an MRI. In other embodiments, the further diagnosis includes, but is not limited to CT scanning, nuclear scanning, bone scanning, PET scans, ultrasounds, X-rays, further tissue biopsies, and surgery.

In other embodiments, after the further diagnosis, the subject is treated with surgery, chemotherapy, radiation, immunotherapy, gene therapy, or a combination thereof. In some embodiments, chemotherapy is given as a treatment for progressive glioblastoma. Such chemotherapy can be selected from the group consisting of alkylating agents, antimetabolites, anthracyclines, topoisomerase inhibitors, or corticosteroids. In another embodiment, the immunotherapy is given as a treatment for progressive glioblastoma. In other embodiments, the immunotherapy is an antibody therapy. In other embodiments, the immunotherapy is either a PD1 antibody therapy or a PD-L1 antibody therapy. In another embodiment, the gene therapy is given as a treatment for progressive glioblastoma. In another embodiment, the gene therapy includes but is not limited to gene editing (i.e., CRISPR-Cas gene editing systems), chimeric antigen receptor (CAR) gene therapy, and gene transfers (i.e., viral transfection and plasmid transformation).

In some embodiments, the biological sample is selected from the group consisting of blood, plasma, serum, cerebrospinal fluid, urine, saliva, stool, synovial fluid, a solid tissue sample, and a cell culture sample.

In one aspect, disclosed herein is a method for monitoring progression in a subject undergoing a treatment for a glioblastoma and adjusting said treatment, the method comprising a) determining an expression status of one or more genes selected from 1000 genes or more isolated from a biological sample from the subject, b) identifying a subject with an expression score greater than 25% as being at risk of having progressing glioblastoma relative to a normal subject, c) giving the subject a first treatment based on the expression score, d) repeating steps a)-d) to monitor a change in the expression score, and e) giving the subject a second treatment if the expression score remains or increases above 25%.

In some embodiments, an expression status of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, or more genes is selected from 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 1751, 1752, 1753, 1754, 1755, 1756, 1757, 1758, 1759, 1760, 1761, 1762, 1763, 1764, 1765, 1766, 1767, 1768, 1769, 1770, 1771, 1772, 1773, 1774, 1775, 1776, 1777, 1778, 1779, 1780, 1781, 1782, 1783, 1784, 1785, 1786, 1787, 1788, 1789, 1790, 1791, 1792, 1793, 1794, 1795, 1796, 1797, 1798, 1799, 1800, 1801, 1802, 1803, 1804, 1805, 1806, 1807, 1808, 1809, 1810, 1811, 1812, 1813, 1814, 1815, 1816, 1817, 1818, 1819, 1820, 1821, 1822, 1823, 1824, 1825, 1826, 1827, 1828, 1829, 1830, 1831, 1832, 1833, 1834, 1835, 1836, 1837, 1838, 1839, 1840, 1841, 1842, 1843, 1844, 1845, 1846, 1847, 1848, 1849, 1850, 1851, 1852, 1853, 1854, 1855, 1856, 1857, 1858, 1859, 1860, 1861, 1862, 1863, 1864, 1865, 1866, 1867, 1868, 1869, 1870, 1871, 1872, 1873, 1874, 1875, 1876, 1877, 1878, 1879, 1880, 1881, 1882, 1883, 1884, 1885, 1886, 1887, 1888, 1889, 1890, 1891, 1892, 1893, 1894, 1895, 1896, 1897, 1898, 1899, 1900, 1901, 1902, 1903, 1904, 1905, 1906, 1907, 1908, 1909, 1910, 1911, 1912, 1913, 1914, 1915, 1916, 1917, 1918, 1919, 1920, 1921, 1922, 1923, 1924, 1925, 1926, 1927, 1928, 1929, 1930, 1931, 1932, 1933, 1934, 1935, 1936, 1937, 1938, 1939, 1940, 1941, 1942, 1943, 1944, 1945, 1946, 1947, 1948, 1949, 1950, 1951, 1952, 1953, 1954, 1955, 1956, 1957, 1958, 1959, 1960, 1961, 1962, 1963, 1964, 1965, 1966, 1967, 1968, 1969, 1970, 1971, 1972, 1973, 1974, 1975, 1976, 1977, 1978, 1979, 1980, 1981, 1982, 1983, 1984, 1985, 1986, 1987, 1988, 1989, 1990, 1991, 1992, 1993, 1994, 1995, 1996, 1997, 1998, 1999, 2000, 2001, 2002, 2003, 2004, 2005, 2006, 2007, 2008, 2009, 2010, 2011, 2012, 2013, 2014, 2015, 2016, 2017, 2018, 2019, 2020, 2021, 2022, 2023, 2024, 2025, 2026, 2027, 2028, 2029, 2030, 2031, 2032, 2033, 2034, 2035, 2036, 2037, 2038, 2039, 2040, 2041, 2042, 2043, 2044, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2097, 2098, 2099, 2100, 2101, 2102, 2103, 2104, 2105, 2106, 2107, 2108, 2109, 2110, 2111, 2112, 2113, 2114, 2115, 2116, 2117, 2118, 2119, 2120, 2121, 2122, 2123, 2124, 2125, 2126, 2127, 2128, 2129, 2130, 2131, 2132, 2133, 2134, 2135, 2136, 2137, 2138, 2139, 2140, 2141, 2142, 2143, 2144, 2145, 2146, 2147, 2148, 2149, 2150, 2151, 2152, 2153, 2154, 2155, 2156, 2157, 2158, 2159, 2160, 2161, 2162, 2163, 2164, 2165, 2166, 2167, 2168, 2169, 2170, 2171, 2172, 2173, 2174, 2175, 2176, 2177, 2178, 2179, 2180, 2181, 2182, 2183, 2184, 2185, 2186, 2187, 2188, 2189, 2190, 2191, 2192, 2193, 2194, 2195, 2196, 2197, 2198, 2199, 2200, 2201, 2202, 2203, 2204, 2205, 2206, 2207, 2208, 2209, 2210, 2211, 2212, 2213, 2214, 2215, 2216, 2217, 2218, 2219, 2220, 2221, 2222, 2223, 2224, 2225, 2226, 2227, 2228, 2229, 2230, 2231, 2232, 2233, 2234, 2235, 2236, 2237, 2238, 2239, 2240, 2241, 2242, 2243, 2244, 2245, 2246, 2247, 2248, 2249, 2250, 2251, 2252, 2253, 2254, 2255, 2256, 2257, 2258, 2259, 2260, 2261, 2262, 2263, 2264, 2265, 2266, 2267, 2268, 2269, 2270, 2271, 2272, 2273, 2274, 2275, 2276, 2277, 2278, 2279, 2280, 2281, 2282, 2283, 2284, 2285, 2286, 2287, 2288, 2289, 2290, 2291, 2292, 2293, 2294, 2295, 2296, 2297, 2298, 2299, 2300, 2301, 2302, 2303, 2304, 2305, 2306, 2307, 2308, 2309, 2310, 2311, 2312, 2313, 2314, 2315, 2316, 2317, 2318, 2319, 2320, 2321, 2322, 2323, 2324, 2325, 2326, 2327, 2328, 2329, 2330, 2331, 2332, 2333, 2334, 2335, 2336, 2337, 2338, 2339, 2340, 2341, 2342, 2343, 2344, 2345, 2346, 2347, 2348, 2349, 2350, 2351, 2352, 2353, 2354, 2355, 2356, 2357, 2358, 2359, 2360, 2361, 2362, 2363, 2364, 2365, 2366, 2367, 2368, 2369, 2370, 2371, 2372, 2373, 2374, 2375, 2376, 2377, 2378, 2379, 2380, 2381, 2382, 2383, 2384, 2385, 2386, 2387, 2388, 2389, 2390, 2391, 2392, 2393, 2394, 2395, 2396, 2397, 2398, 2399, 2400, 2401, 2402, 2403, 2404, 2405, 2406, 2407, 2408, 2409, 2410, 2411, 2412, 2413, 2414, 2415, 2416, 2417, 2418, 2419, 2420, 2421, 2422, 2423, 2424, 2425, 2426, 2427, 2428, 2429, 2430, 2431, 2432, 2433, 2434, 2435, 2436, 2437, 2438, 2439, 2440, 2441, 2442, 2443, 2444, 2445, 2446, 2447, 2448, 2449, 2450, 2451, 2452, 2453, 2454, 2455, 2456, 2457, 2458, 2459, 2460, 2461, 2462, 2463, 2464, 2465, 2466, 2467, 2468, 2469, 2470, 2471, 2472, 2473, 2474, 2475, 2476, 2477, 2478, 2479, 2480, 2481, 2482, 2483, 2484, 2485, 2486, 2487, 2488, 2489, 2490, 2491, 2492, 2493, 2494, 2495, 2496, 2497, 2498, 2499, 2500, 2501, 2502, 2503, 2504, 2505, 2506, 2507, 2508, 2509, 2510, 2511, 2512, 2513, 2514, 2515, 2516, 2517, 2518, 2519, 2520, 2521, 2522, 2523, 2524, 2525, 2526, 2527, 2528, 2529, 2530, 2531, 2532, 2533, 2534, 2535, 2536, 2537, 2538, 2539, 2540, 2541, 2542, 2543, 2544, 2545, 2546, 2547, 2548, 2549, 2550, 2551, 2552, 2553, 2554, 2555, 2556, 2557, 2558, 2559, 2560, 2561, 2562, 2563, 2564, 2565, 2566, 2567, 2568, 2569, 2570, 2571, 2572, 2573, 2574, 2575, 2576, 2577, 2578, 2579, 2580, 2581, 2582, 2583, 2584, 2585, 2586, 2587, 2588, 2589, 2590, 2591, 2592, 2593, 2594, 2595, 2596, 2597, 2598, 2599, 2600, 2601, 2602, 2603, 2604, 2605, 2606, 2607, 2608, 2609, 2610, 2611, 2612, 2613, 2614, 2615, 2616, 2617, 2618, 2619, 2620, 2621, 2622, 2623, 2624, 2625, 2626, 2627, 2628, 2629, 2630, 2631, 2632, 2633, 2634, 2635, 2636, 2637, 2638, 2639, 2640, 2641, 2642, 2643, 2644, 2645, 2646, 2647, 2648, 2649, 2650, 2651, 2652, 2653, 2654, 2655, 2656, 2657, 2658, 2659, 2660, 2661, 2662, 2663, 2664, 2665, 2666, 2667, 2668, 2669, 2670, 2671, 2672, 2673, 2674, 2675, 2676, 2677, 2678, 2679, 2680, 2681, 2682, 2683, 2684, 2685, 2686, 2687, 2688, 2689, 2690, 2691, 2692, 2693, 2694, 2695, 2696, 2697, 2698, 2699, 2700, 2701, 2702, 2703, 2704, 2705, 2706, 2707, 2708, 2709, 2710, 2711, 2712, 2713, 2714, 2715, 2716, 2717, 2718, 2719, 2720, 2721, 2722, 2723, 2724, 2725, 2726, 2727, 2728, 2729, 2730, 2731, 2732, 2733, 2734, 2735, 2736, 2737, 2738, 2739, 2740, 2741, 2742, 2743, 2744, 2745, 2746, 2747, 2748, 2749, 2750, 2751, 2752, 2753, 2754, 2755, 2756, 2757, 2758, 2759, 2760, 2761, 2762, 2763, 2764, 2765, 2766, 2767, 2768, 2769, 2770, 2771, 2772, 2773, 2774, 2775, 2776, 2777, 2778, 2779, 2780, 2781, 2782, 2783, 2784, 2785, 2786, 2787, 2788, 2789, 2790, 2791, 2792, 2793, 2794, 2795, 2796, 2797, 2798, 2799, 2800, 2801, 2802, 2803, 2804, 2805, 2806, 2807, 2808, 2809, 2810, 2811, 2812, 2813, 2814, 2815, 2816, 2817, 2818, 2819, 2820, 2821, 2822, 2823, 2824, 2825, 2826, 2827, 2828, 2829, 2830, 2831, 2832, 2833, 2834, 2835, 2836, 2837, 2838, 2839, 2840, 2841, 2842, 2843, 2844, 2845, 2846, 2847, 2848, 2849, 2850, 2851, 2852, 2853, 2854, 2855, 2856, 2857, 2858, 2859, 2860, 2861, 2862, 2863, 2864, 2865, 2866, 2867, 2868, 2869, 2870, 2871, 2872, 2873, 2874, 2875, 2876, 2877, 2878, 2879, 2880, 2881, 2882, 2883, 2884, 2885, 2886, 2887, 2888, 2889, 2890, 2891, 2892, 2893, 2894, 2895, 2896, 2897, 2898, 2899, 2900, 2901, 2902, 2903, 2904, 2905, 2906, 2907, 2908, 2909, 2910, 2911, 2912, 2913, 2914, 2915, 2916, 2917, 2918, 2919, 2920, 2921, 2922, 2923, 2924, 2925, 2926, 2927, 2928, 2929, 2930, 2931, 2932, 2933, 2934, 2935, 2936, 2937, 2938, 2939, 2940, 2941, 2942, 2943, 2944, 2945, 2946, 2947, 2948, 2949, 2950, 2951, 2952, 2953, 2954, 2955, 2956, 2957, 2958, 2959, 2960, 2961, 2962, 2963, 2964, 2965, 2966, 2967, 2968, 2969, 2970, 2971, 2972, 2973, 2974, 2975, 2976, 2977, 2978, 2979, 2980, 2981, 2982, 2983, 2984, 2985, 2986, 2987, 2988, 2989, 2990, 2991, 2992, 2993, 2994, 2995, 2996, 2997, 2998, 2999, 3000, 3001, 3002, 3003, 3004, 3005, 3006, 3007, 3008, 3009, 3010, 3011, 3012, 3013, 3014, 3015, 3016, 3017, 3018, 3019, 3020, 3021, 3022, 3023, 3024, 3025, 3026, 3027, 3028, 3029, 3030, 3031, 3032, 3033, 3034, 3035, 3036, 3037, 3038, 3039, 3040, 3041, 3042, 3043, 3044, 3045, 3046, 3047, 3048, 3049, 3050, 3051, 3052, 3053, 3054, 3055, 3056, 3057, 3058, 3059, 3060, 3061, 3062, 3063, 3064, 3065, 3066, 3067, 3068, 3069, 3070, 3071, 3072, 3073, 3074, 3075, 3076, 3077, 3078, 3079, 3080, 3081, 3082, 3083, 3084, 3085, 3086, 3087, 3088, 3089, 3090, 3091, 3092, 3093, 3094, 3095, 3096, 3097, 3098, 3099, 3100, 3101, 3102, 3103, 3104, 3105, 3106, 3107, 3108, 3109, 3110, 3111, 3112, 3113, 3114, 3115, 3116, 3117, 3118, 3119, 3120, 3121, 3122, 3123, 3124, 3125, 3126, 3127, 3128, 3129, 3130, 3131, 3132, 3133, 3134, 3135, 3136, 3137, 3138, 3139, 3140, 3141, 3142, 3143, 3144, 3145, 3146, 3147, 3148, 3149, 3150, 3151, 3152, 3153, 3154, 3155, 3156, 3157, 3158, 3159, 3160, 3161, 3162, 3163, 3164, 3165, 3166, 3167, 3168, 3169, 3170, 3171, 3172, 3173, 3174, 3175, 3176, 3177, 3178, 3179, 3180, 3181, 3182, 3183, 3184, 3185, 3186, 3187, 3188, 3189, 3190, 3191, 3192, 3193, 3194, 3195, 3196, 3197, 3198, 3199, 3200, 3201, 3202, 3203, 3204, 3205, 3206, 3207, 3208, 3209, 3210, 3211, 3212, 3213, 3214, 3215, 3216, 3217, 3218, 3219, 3220, 3221, 3222, 3223, 3224, 3225, 3226, 3227, 3228, 3229, 3230, 3231, 3232, 3233, 3234, 3235, 3236, 3237, 3238, 3239, 3240, 3241, 3242, 3243, 3244, 3245, 3246, 3247, 3248, 3249, 3250, 3251, 3252, 3253, 3254, 3255, 3256, 3257, 3258, 3259, 3260, 3261, 3262, 3263, 3264, 3265, 3266, 3267, 3268, 3269, 3270, 3271, 3272, 3273, 3274, 3275, 3276, 3277, 3278, 3279, 3280, 3281, 3282, 3283, 3284, 3285, 3286, 3287, 3288, 3289, 3290, 3291, 3292, 3293, 3294, 3295, 3296, 3297, 3298, 3299, 3300, 3301, 3302, 3303, 3304, 3305, 3306, 3307, 3308, 3309, 3310, 3311, 3312, 3313, 3314, 3315, 3316, 3317, 3318, 3319, 3320, 3321, 3322, 3323, 3324, 3325, 3326, 3327, 3328, 3329, 3330, 3331, 3332, 3333, 3334, 3335, 3336, 3337, 3338, 3339, 3340, 3341, 3342, 3343, 3344, 3345, 3346, 3347, 3348, 3349, 3350, 3351, 3352, 3353, 3354, 3355, 3356, 3357, 3358, 3359, 3360, 3361, 3362, 3363, 3364, 3365, 3366, 3367, 3368, 3369, 3370, 3371, 3372, 3373, 3374, 3375, 3376, 3377, 3378, 3379, 3380, 3381, 3382, 3383, 3384, 3385, 3386, 3387, 3388, 3389, 3390, 3391, 3392, 3393, 3394, 3395, 3396, 3397, 3398, 3399, 3400, 3401, 3402, 3403, 3404, 3405, 3406, 3407, 3408, 3409, 3410, 3411, 3412, 3413, 3414, 3415, 3416, 3417, 3418, 3419, 3420, 3421, 3422, 3423, 3424, 3425, 3426, 3427, 3428, 3429, 3430, 3431, 3432, 3433, 3434, 3435, 3436, 3437, 3438, 3439, 3440, 3441, 3442, 3443, 3444, 3445, 3446, 3447, 3448, 3449, 3450, 3451, 3452, 3453, 3454, 3455, 3456, 3457, 3458, 3459, 3460, 3461, 3462, 3463, 3464, 3465, 3466, 3467, 3468, 3469, 3470, 3471, 3472, 3473, 3474, 3475, 3476, 3477, 3478, 3479, 3480, 3481, 3482, 3483, 3484, 3485, 3486, 3487, 3488, 3489, 3490, 3491, 3492, 3493, 3494, 3495, 3496, 3497, 3498, 3499, 3500, 3501, 3502, 3503, 3504, 3505, 3506, 3507, 3508, 3509, 3510, 3511, 3512, 3513, 3514, 3515, 3516, 3517, 3518, 3519, 3520, 3521, 3522, 3523, 3524, 3525, 3526, 3527, 3528, 3529, 3530, 3531, 3532, 3533, 3534, 3535, 3536, 3537, 3538, 3539, 3540, 3541, 3542, 3543, 3544, 3545, 3546, 3547, 3548, 3549, 3550, 3551, 3552, 3553, 3554, 3555, 3556, 3557, 3558, 3559, 3560, 3561, 3562, 3563, 3564, 3565, 3566, 3567, 3568, 3569, 3570, 3571, 3572, 3573, 3574, 3575, 3576, 3577, 3578, 3579, 3580, 3581, 3582, 3583, 3584, 3585, 3586, 3587, 3588, 3589, 3590, 3591, 3592, 3593, 3594, 3595, 3596, 3597, 3598, 3599, 3600, 3601, 3602, 3603, 3604, 3605, 3606, 3607, 3608, 3609, 3610, 3611, 3612, 3613, 3614, 3615, 3616, 3617, 3618, 3619, 3620, 3621, 3622, 3623, 3624, 3625, 3626, 3627, 3628, 3629, 3630, 3631, 3632, 3633, 3634, 3635, 3636, 3637, 3638, 3639, 3640, 3641, 3642, 3643, 3644, 3645, 3646, 3647, 3648, 3649, 3650, 3651, 3652, 3653, 3654, 3655, 3656, 3657, 3658, 3659, 3660, 3661, 3662, 3663, 3664, 3665, 3666, 3667, 3668, 3669, 3670, 3671, 3672, 3673, 3674, 3675, 3676, 3677, 3678, 3679, 3680, 3681, 3682, 3683, 3684, 3685, 3686, 3687, 3688, 3689, 3690, 3691, 3692, 3693, 3694, 3695, 3696, 3697, 3698, 3699, 3700, 3701, 3702, 3703, 3704, 3705, 3706, 3707, 3708, 3709, 3710, 3711, 3712, 3713, 3714, 3715, 3716, 3717, 3718, 3719, 3720, 3721, 3722, 3723, 3724, 3725, 3726, 3727, 3728, 3729, 3730, 3731, 3732, 3733, 3734, 3735, 3736, 3737, 3738, 3739, 3740, 3741, 3742, 3743, 3744, 3745, 3746, 3747, 3748, 3749, 3750, 3751, 3752, 3753, 3754, 3755, 3756, 3757, 3758, 3759, 3760, 3761, 3762, 3763, 3764, 3765, 3766, 3767, 3768, 3769, 3770, 3771, 3772, 3773, 3774, 3775, 3776, 3777, 3778, 3779, 3780, 3781, 3782, 3783, 3784, 3785, 3786, 3787, 3788, 3789, 3790, 3791, 3792, 3793, 3794, 3795, 3796, 3797, 3798, 3799, 3800, 3801, 3802, 3803, 3804, 3805, 3806, 3807, 3808, 3809, 3810, 3811, 3812, 3813, 3814, 3815, 3816, 3817, 3818, 3819, 3820, 3821, 3822, 3823, 3824, 3825, 3826, 3827, 3828, 3829, 3830, 3831, 3832, 3833, 3834, 3835, 3836, 3837, 3838, 3839, 3840, 3841, 3842, 3843, 3844, 3845, 3846, 3847, 3848, 3849, 3850, 3851, 3852, 3853, 3854, 3855, 3856, 3857, 3858, 3859, 3860, 3861, 3862, 3863, 3864, 3865, 3866, 3867, 3868, 3869, 3870, 3871, 3872, 3873, 3874, 3875, 3876, 3877, 3878, 3879, 3880, 3881, 3882, 3883, 3884, 3885, 3886, 3887, 3888, 3889, 3890, 3891, 3892, 3893, 3894, 3895, 3896, 3897, 3898, 3899, 3900, 3901, 3902, 3903, 3904, 3905, 3906, 3907, 3908, 3909, 3910, 3911, 3912, 3913, 3914, 3915, 3916, 3917, 3918, 3919, 3920, 3921, 3922, 3923, 3924, 3925, 3926, 3927, 3928, 3929, 3930, 3931, 3932, 3933, 3934, 3935, 3936, 3937, 3938, 3939, 3940, 3941, 3942, 3943, 3944, 3945, 3946, 3947, 3948, 3949, 3950, 3951, 3952, 3953, 3954, 3955, 3956, 3957, 3958, 3959, 3960, 3961, 3962, 3963, 3964, 3965, 3966, 3967, 3968, 3969, 3970, 3971, 3972, 3973, 3974, 3975, 3976, 3977, 3978, 3979, 3980, 3981, 3982, 3983, 3984, 3985, 3986, 3987, 3988, 3989, 3990, 3991, 3992, 3993, 3994, 3995, 3996, 3997, 3998, 3999, 4000, 4001, 4002, 4003, 4004, 4005, 4006, 4007, 4008, 4009, 4010, 4011, 4012, 4013, 4014, 4015, 4016, 4017, 4018, 4019, 4020, 4021, 4022, 4023, 4024, 4025, 4026, 4027, 4028, 4029, 4030, 4031, 4032, 4033, 4034, 4035, 4036, 4037, 4038, 4039, 4040, 4041, 4042, 4043, 4044, 4045, 4046, 4047, 4048, 4049, 4050, 4051, 4052, 4053, 4054, 4055, 4056, 4057, 4058, 4059, 4060, 4061, 4062, 4063, 4064, 4065, 4066, 4067, 4068, 4069, 4070, 4071, 4072, 4073, 4074, 4075, 4076, 4077, 4078, 4079, 4080, 4081, 4082, 4083, 4084, 4085, 4086, 4087, 4088, 4089, 4090, 4091, 4092, 4093, 4094, 4095, 4096, 4097, 4098, 4099, 4100, 4101, 4102, 4103, 4104, 4105, 4106, 4107, 4108, 4109, 4110, 4111, 4112, 4113, 4114, 4115, 4116, 4117, 4118, 4119, 4120, 4121, 4122, 4123, 4124, 4125, 4126, 4127, 4128, 4129, 4130, 4131, 4132, 4133, 4134, 4135, 4136, 4137, 4138, 4139, 4140, 4141, 4142, 4143, 4144, 4145, 4146, 4147, 4148, 4149, 4150, 4151, 4152, 4153, 4154, 4155, 4156, 4157, 4158, 4159, 4160, 4161, 4162, 4163, 4164, 4165, 4166, 4167, 4168, 4169, 4170, 4171, 4172, 4173, 4174, 4175, 4176, 4177, 4178, 4179, 4180, 4181, 4182, 4183, 4184, 4185, 4186, 4187, 4188, 4189, 4190, 4191, 4192, 4193, 4194, 4195, 4196, 4197, 4198, 4199, 4200, 4201, 4202, 4203, 4204, 4205, 4206, 4207, 4208, 4209, 4210, 4211, 4212, 4213, 4214, 4215, 4216, 4217, 4218, 4219, 4220, 4221, 4222, 4223, 4224, 4225, 4226, 4227, 4228, 4229, 4230, 4231, 4232, 4233, 4234, 4235, 4236, 4237, 4238, 4239, 4240, 4241, 4242, 4243, 4244, 4245, 4246, 4247, 4248, 4249, 4250, 4251, 4252, 4253, 4254, 4255, 4256, 4257, 4258, 4259, 4260, 4261, 4262, 4263, 4264, 4265, 4266, 4267, 4268, 4269, 4270, 4271, 4272, 4273, 4274, 4275, 4276, 4277, 4278, 4279, 4280, 4281, 4282, 4283, 4284, 4285, 4286, 4287, 4288, 4289, 4290, 4291, 4292, 4293, 4294, 4295, 4296, 4297, 4298, 4299, 4300, 4301, 4302, 4303, 4304, 4305, 4306, 4307, 4308, 4309, 4310, 4311, 4312, 4313, 4314, 4315, 4316, 4317, 4318, 4319, 4320, 4321, 4322, 4323, 4324, 4325, 4326, 4327, 4328, 4329, 4330, 4331, 4332, 4333, 4334, 4335, 4336, 4337, 4338, 4339, 4340, 4341, 4342, 4343, 4344, 4345, 4346, 4347, 4348, 4349, 4350, 4351, 4352, 4353, 4354, 4355, 4356, 4357, 4358, 4359, 4360, 4361, 4362, 4363, 4364, 4365, 4366, 4367, 4368, 4369, 4370, 4371, 4372, 4373, 4374, 4375, 4376, 4377, 4378, 4379, 4380, 4381, 4382, 4383, 4384, 4385, 4386, 4387, 4388, 4389, 4390, 4391, 4392, 4393, 4394, 4395, 4396, 4397, 4398, 4399, 4400, 4401, 4402, 4403, 4404, 4405, 4406, 4407, 4408, 4409, 4410, 4411, 4412, 4413, 4414, 4415, 4416, 4417, 4418, 4419, 4420, 4421, 4422, 4423, 4424, 4425, 4426, 4427, 4428, 4429, 4430, 4431, 4432, 4433, 4434, 4435, 4436, 4437, 4438, 4439, 4440, 4441, 4442, 4443, 4444, 4445, 4446, 4447, 4448, 4449, 4450, 4451, 4452, 4453, 4454, 4455, 4456, 4457, 4458, 4459, 4460, 4461, 4462, 4463, 4464, 4465, 4466, 4467, 4468, 4469, 4470, 4471, 4472, 4473, 4474, 4475, 4476, 4477, 4478, 4479, 4480, 4481, 4482, 4483, 4484, 4485, 4486, 4487, 4488, 4489, 4490, 4491, 4492, 4493, 4494, 4495, 4496, 4497, 4498, 4499, 4500, 4501, 4502, 4503, 4504, 4505, 4506, 4507, 4508, 4509, 4510, 4511, 4512, 4513, 4514, 4515, 4516, 4517, 4518, 4519, 4520, 4521, 4522, 4523, 4524, 4525, 4526, 4527, 4528, 4529, 4530, 4531, 4532, 4533, 4534, 4535, 4536, 4537, 4538, 4539, 4540, 4541, 4542, 4543, 4544, 4545, 4546, 4547, 4548, 4549, 4550, 4551, 4552, 4553, 4554, 4555, 4556, 4557, 4558, 4559, 4560, 4561, 4562, 4563, 4564, 4565, 4566, 4567, 4568, 4569, 4570, 4571, 4572, 4573, 4574, 4575, 4576, 4577, 4578, 4579, 4580, 4581, 4582, 4583, 4584, 4585, 4586, 4587, 4588, 4589, 4590, 4591, 4592, 4593, 4594, 4595, 4596, 4597, 4598, 4599, 4600, 4601, 4602, 4603, 4604, 4605, 4606, 4607, 4608, 4609, 4610, 4611, 4612, 4613, 4614, 4615, 4616, 4617, 4618, 4619, 4620, 4621, 4622, 4623, 4624, 4625, 4626, 4627, 4628, 4629, 4630, 4631, 4632, 4633, 4634, 4635, 4636, 4637, 4638, 4639, 4640, 4641, 4642, 4643, 4644, 4645, 4646, 4647, 4648, 4649, 4650, 4651, 4652, 4653, 4654, 4655, 4656, 4657, 4658, 4659, 4660, 4661, 4662, 4663, 4664, 4665, 4666, 4667, 4668, 4669, 4670, 4671, 4672, 4673, 4674, 4675, 4676, 4677, 4678, 4679, 4680, 4681, 4682, 4683, 4684, 4685, 4686, 4687, 4688, 4689, 4690, 4691, 4692, 4693, 4694, 4695, 4696, 4697, 4698, 4699, 4700, 4701, 4702, 4703, 4704, 4705, 4706, 4707, 4708, 4709, 4710, 4711, 4712, 4713, 4714, 4715, 4716, 4717, 4718, 4719, 4720, 4721, 4722, 4723, 4724, 4725, 4726, 4727, 4728, 4729, 4730, 4731, 4732, 4733, 4734, 4735, 4736, 4737, 4738, 4739, 4740, 4741, 4742, 4743, 4744, 4745, 4746, 4747, 4748, 4749, 4750, 4751, 4752, 4753, 4754, 4755, 4756, 4757, 4758, 4759, 4760, 4761, 4762, 4763, 4764, 4765, 4766, 4767, 4768, 4769, 4770, 4771, 4772, 4773, 4774, 4775, 4776, 4777, 4778, 4779, 4780, 4781, 4782, 4783, 4784, 4785, 4786, 4787, 4788, 4789, 4790, 4791, 4792, 4793, 4794, 4795, 4796, 4797, 4798, 4799, 4800, 4801, 4802, 4803, 4804, 4805, 4806, 4807, 4808, 4809, 4810, 4811, 4812, 4813, 4814, 4815, 4816, 4817, 4818, 4819, 4820, 4821, 4822, 4823, 4824, 4825, 4826, 4827, 4828, 4829, 4830, 4831, 4832, 4833, 4834, 4835, 4836, 4837, 4838, 4839, 4840, 4841, 4842, 4843, 4844, 4845, 4846, 4847, 4848, 4849, 4850, 4851, 4852, 4853, 4854, 4855, 4856, 4857, 4858, 4859, 4860, 4861, 4862, 4863, 4864, 4865, 4866, 4867, 4868, 4869, 4870, 4871, 4872, 4873, 4874, 4875, 4876, 4877, 4878, 4879, 4880, 4881, 4882, 4883, 4884, 4885, 4886, 4887, 4888, 4889, 4890, 4891, 4892, 4893, 4894, 4895, 4896, 4897, 4898, 4899, 4900, 4901, 4902, 4903, 4904, 4905, 4906, 4907, 4908, 4909, 4910, 4911, 4912, 4913, 4914, 4915, 4916, 4917, 4918, 4919, 4920, 4921, 4922, 4923, 4924, 4925, 4926, 4927, 4928, 4929, 4930, 4931, 4932, 4933, 4934, 4935, 4936, 4937, 4938, 4939, 4940, 4941, 4942, 4943, 4944, 4945, 4946, 4947, 4948, 4949, 4950, 4951, 4952, 4953, 4954, 4955, 4956, 4957, 4958, 4959, 4960, 4961, 4962, 4963, 4964, 4965, 4966, 4967, 4968, 4969, 4970, 4971, 4972, 4973, 4974, 4975, 4976, 4977, 4978, 4979, 4980, 4981, 4982, 4983, 4984, 4985, 4986, 4987, 4988, 4989, 4990, 4991, 4992, 4993, 4994, 4995, 4996, 4997, 4998, 4999, 5000, or more genes isolated from a biological sample. In another embodiment, the subject is identified with an expression score of 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as being at risk of having progressing glioblastoma relative to a normal subject.

In another embodiment, the expression status is determined from any one gene including but not limited to AC005592.2, AC007879.7. ACTA2, ACTG2, ADAMS, ADM, ANGPTL4, ANKRD1, ANPEP, AOC3, APCDD1L, ATF5, ATP2A3, ATP6VOD2, B3GNT7, BCL3, C15orf48, CA9, CCIN, CCR3, CCR7, CD1C, CD300E, CLCF1, CLDN23, CNN2, COL1A1, COL1A2, COL3A1, COL5A1, COL6A3, CSTA, CXCR4, CYR61, DMBX1, DPP4, EGLN3, EMILIN2, FAM46B. FN1, FOSL1, FOSL2, FPR2, GALNT5, GPR18, HBEGF, HOXB6, HOXB7, ICAM1, IL1R1, IL7R, IL8, IRF1, ITK, KLF4, LCK, LIF, LOX, LUCAT1, MAFF, MAPK13, MEDAG, MEFV, MMP11, MMP9, NABP1, NAMPT, NDRG1, NFKB1A, NLRP12, P4HA3, PAX8, PDLIM1, PIM1, PLAUR, PLEKHS1, PPP1R15A, PTGER2, RAB43, RAC2, RGS1, RGS2, RHOH, RND3, ROR2, RP11-1149O23.2, RP11-1149O23.3, RP11-290F20.3, RP11-59D5_B.2, RP5-1021I20.2, S100A8, S100A9, SDC1, SERPINE1, SLC16A3, SNAI1, SPOCD1, SUSD2, THBD, THBS1, TMEM217, TNFAIP6, TNFAIP8, TNFRSF10D, TREM1, TREML2, TREML3P, VDR, C3H12A, ABCA6, AC002059.10, AC005932.1, AC008060.7, AC011558.5, AC017076.5, AC098823.3, ACSM5, ACTA2-AS1, ADRA2B, APLNR, ARHGEF5, ARRDC2, BEST1, BHLHE41, CD55, CD83, CDK18, CLCA2, CLMN, COL4A5, CPXM2, CTC-1337H24.1, CTD-2034121.2, CXorf65, CYB5R2, DNM1P47, DYSF, EHF, ELOVL1, EVI2A, FAM124A, FAM53B, FGF20, FKBP11, FMN1, GALNT6, GPR37, GTSF1, HMSD, HOXD-AS1, HPN-AS1, ICOSLG, IL9R, IP6K3, KANK4, KCNJ2-AS1, KCNN4, KEL, LAG3, LHB, LINC01010, LRMP, LRP2, LRRC63, LURAP1, MFSD6L, MGST1, MIR24-2, MS4A4E, NFKBID, NME8, OSTF1, P2RX1, PATL2, PCP2, PCSK6, PDLIM7, PLA2G7, PLD1, PM20D1, PPM1N, PRG4, RP11-1094M14.8, RP11-136K7.2, RP11-161H23.5, RP11-16K12.1, RP11-350G8.5, RP11-399019.9, RP11-434H6.6, RP11-597D13.7, RP11-700H6.1, RP11-703I16.1, RP11-883G14.1, RP3-47704.14, RP5-943J3.1, RP6-99M1.2, RPS10P7, SAMD3, SEPP1, SH2D3A, SLC45A3, SLC52A1, ST18, SVIP, TM6SF1, TMC7, TMEM139, TMPRSS13, TPM2, TRIM59, TRPC2, TRPM6, TRPV5, U91328.20, UCN2, ABCG1, ABI3BP, AC003003.5, AC004988.1, AC005082.12, AC006129.1, AC006129.2, AC011899.9, AC026202.3, ACAT2, ACP5, ACSL1, ADAM28, ADAMTS1, ADAMTS12, ADAP2, ADRB2, AF127936.5, AKR1B10, AKR1B15, ALOX12B, ALOX5, ALOX5AP, AMICA1, AMPD3, ANKRD22, AOAH, AP000476.1, AP000783.1, APBB1IP, APH1B, APOBEC3C, APOBR, ARHGAP30, ARHGDIB, ARHGEF34P, ARHGEF35, ARL11, ARL3, ARRDC4, ASB12, ATP8B4, AXL, B4GALT1, BCL2A1, BCR, BIN2, BIRC3, BMP2K, BTK, C10orf10, C11orf21, C11orf45, C16orf54, C16orf92, C19orf35, C1QC, C2, C3AR1, C4BPB, C5AR1, C5AR2, C9orf47, C9orf66, CABP7, CACNA2D4, CASP1, CASP10, CASP5, CASS4, CBFA2T3, CCL18, CCL2, CCL20, CCL22, CCL8, CCR1, CCR2, CCR4, CCR5, CCRL2, CD14, CD163, CD180, CD1D, CD200R1, CD274, CD28, CD300A, CD300C, CD300LB, CD300LF, CD3G, CD4, CD40, CD40LG, CD53, CD59, CD68, CD69, CD80, CD84, CD86, CD96, CDCP1, CDKN1A, CEACAM21, CEACAM4, CEBPA, CECR1, CELSR3, CFH, CFLAR, CHCHD7, CHRM3-AS2, CIITA, CLEC10A, CLEC12A, CLEC12B, CLEC2B, CLEC7A, CLSTN3, CMAHP, CMKLR1, CMPK2, CNN1, COMP, COQ3, CP, CPA6, CPM, CR1, CREG1, CRTAM, CSF1R, CSF2RB, CSF3R, CTC-205M6.5, CTC-250114.6, CTD-2258A20.S, CTD-2306M10.1, CTD-2540B15.7, CTD-2587H19.1, CTSB, CTSC, CTSH, CTSL, CTSS, CTSZ, CXCL12, CXCL16, CXCL6, CXCR2P1, CXCR6, CXorf21, CYBB, CYP19A1, CYTH4, CYTIP, DAB2, DAPP1, DCN, DCSTAMP, DDO, DDX58, DEC1, DENND3, DLGAP1-AS3, DLX2, DLX6, DLX6-AS1, DMRTC1B, DNAJC5B, DNASE2B, DOCK2, DOCK8, DOK3, DPY19L2P4, DRAM1, DSC2, DUSP1, EFCAB4B, ELL2, EMB, EMR1, EMR2, EMR4P, EPSTI1, EVI2B, F13A1, FAM105A FAM177B, FAM20A, FAM213A, FAM57B, FASLG, FAXC, FBP1, FBXO32, FCER1A, FCER1G, FCGBP, FCGR2A, FCGR2B, FCGR3A, FCRL6, FFAR4, FGD2, FGL2, FGR, FH, FLVCR2, FMO2, FMO4, FMOD, FOXD4, FPR1, FPR3, FSD1, FUCA1, FUT9, GAB3, GADD45B, GAL3ST4, GALNT12, GAPT, GATA3, GBP1P1, GBP2, GBP3, GCH1, GCNT1, GDAP1L1, GDI1, GIMAP2, GIMAP4, GIMAP6, GIMAP7, GIMAP8, GLIPR1, GLRX, GM2A, GNA15, GPNMB, GPR132, GPR141, GPR160, GPR183, GPR34, GPR65, GPR82, GPR84, GPR85, GPRIN3, GRM8, GSK3A, GVINP1, GVQW1, HAVCR2, HCK, HCLS1, HCN4, HLA-DMA, HLA-DMB, HLA-DOA, HLA-DPA1, HLA-DQA2, HLA-DRA, HNMT, HPGDS, HPSE, HTR2B, HTRA4, IFI30, IFI44L, IFIH1, IFIT3, IGF1, IGSF6, IKBKE, IKZF1, IL10, IL10RA, IL12RB1, IL15RA, IL16, IL17RA, IL18BP, IL1A, IL1B, IL1RN, IL21R, IL2RA, IL2RB, IL2RG, IL4I1, IL4R, IL6, ILDR1, IRAK3, IRF4, IRF5, IRF8, IRF9, ITGAL, ITGAM, ITGAX, ITGB2, JPH2, KCNF1, KCNJ5, KCNK13, KCNMB1, KCNQ1, KLC2, KLHDC7B, KLHL6, KLRC1, KLRD1, KMO, KYNU, LA16c-380H5.5, LACTB, LAIR1, LAMP1, LANCL2, LAPTMS, LAT2, LAYN, LBP, LCP1, LCP2, LGALS17A, LILRA1, LILRA6, LILRB1, LILRB2, LILRB4, LILRB5, LINC00539, LINC00578, LINC00702, LINC00968, LPAR5, LPCAT2, LPXN, LRRC25, LRRC39, LRRTM1, LTBP2, LTBR, LUM, LURAP1L, LY9, LYN, LYSMD2, LYVE1, LYZ, M6PR, MAF, MAFB, MALL, MAN1A1, MAN1C1, MAOB, MAP3K8, MCOLN2, MERTK, MGAT4A, MGAT5B, MITF, MKNK1, MLKL, MLPH, MMD2, MNDA, MPEG1, MRC1, MRC1L1, MS4A4A, MS4A6A, MS4A7, MSR1, MX1, MX2, MYO1F, NAAA, NCF2, NCKAP1L, NDUFA6-AS1, NFAM1, NFATC2, NGFR, NGFRAP1, NLRC4, NPL, NRROS, NUDT16P, OAS1, OAS2, OLFML2B, OLFML3, OLR1, OSM, OTUD1, P2RY13, P2RY6, P2RY8, PALD1, PALMD, PAPPA, PARP14, PARP15, PCED1B, PDCD1LG2, PDZD4, PGA5, PIK3AP1, PIK3CD, PIK3R5, PIRT, PJA1, PLA2G15, PLB1, PLBD1, PLCG2, PLEK, PLEKHA3P1, PLEKHO2, PLXDC2, POU2F2, PPARG, PPP1R14C, PPP1R17, PRDM12, PRKCD, PRLR, PRRT4, PTAFR, PTCHD2, PTGDR, PTGER4, PTGFR, PTPN22, PTPN7, PTPRC, PTX3, PYHIN1, RAB20, RAB38, RAB44, RAD51B, RARRES1, RASGRP4, RASL11B, RBM47, RERGL, RGS18, RHBDF2, RHOBTB2, RIN3, RIPK3, RNASE2, RNASE3, RNF112, RNF144B, RP11-1024P17.1, RP11-10K16.1, RP11-1149M10.2, RP11-117D22.2, RP11-119F19.2, RP11-121A8.1, RP11-153M7.1, RP11-153M7.3, RP11-196G18.3, RP11-23P13.6, RP11-245J24.1, RP11-271K11.5, RP11-307C19.1, RP11-325F22.2, RP11-346C20.4, RP11-353N4.5, RP11-357K6.1, RP11-363E6.3, RP11-364B14.3, RP11-379B8.1, RP11-384L8.1, RP11-3B12.1, RP11-403P17.5, RP11-439A17.9, RP11-477J21.6, RP11-47122.3, RP11-497H16.7, RP11-523018.5, RP11-565J7.1, RP11-588H23.3, RP11-626G11.4, RP11-676J15.1, RP11-70C1.1, RP11-733018.1, RP11-750H9.5, RP11-760N9.1, RP11-792D21.2, RP11-838N2.4, RP11-84C10.2, RP11-861A13.4, RP11-90P13.1, RP11-93B14.5, RP11-960L18.1, RP11-986E7.7, RP4-555D20.4, RP4-60717.1, RP5-1043L13.1, RP5-1070A16.1, RP5-998N21.4, RPS6KA1, RSAD2, RUSC2, S100Z, SAMSN1, SASH3, SCG3, SCIMP, SCIN, SELE, SERPINA1, SERPINA3, SERPINA5, SERPINB1, SEZ6, SGMS2, SHANK3, SHH, SHISA6, SIGLEC1, SIGLEC10, SIGLEC11, SIGLEC17P, SIGLEC7, SIGLEC9, SIRPB2, SLA, SLAMF1, SLAMF6, SLAMF7, SLAMF8, SLC11A1, SLC16A5, SLC25A22, SLC2A5, SLC2A9, SLC46A2, SLC46A3, SLC7A7, SLC9A9, SLCO2B1, SLFN12L, SMPD3, SMPDL3A, SNAP25-AS1, SNX20, SP110, SP140, SPATA12, SPATC1, SPINT1, SPN, SPP1, SPTBN2, SQRDL, SRRM3, SSTR5, ST14, ST6GAL1, ST8SIA4, STAP1, STARD5, STAT5A, STEAP1B, STEAP4, STX11, STX1B, SULF1, SUSD1, SUSD4, SYK, SYT3, SYTL3, TACSTD2, TAGAP, TAGLN, TAGLN3, TBC1D2B, TBX19, TEC, TERC, TFEC, TGFBR2, TGM2, THBS2, THEMIS, THEMIS2, TIFAB, TLR1, TLR10, TLR2, TLR4, TLR5, TLR6, TLR7, TLR8, TMBIM1, TMC8, TMEM106A, TMEM110, TMEM140, TMEM150B, TMEM173, TMEM176B. TMEM86A, TNFRSF1B, TNFSF13, TNFSF13B, TNFSF15, TNFSF8, TPTE2P1, TRAPPC3L, TRIM22, TRIM38, TRNP1, TRPV4, TSLP, TTC7A, UCP2, USP43, VAMP3, VEGFC, VNN1, VNN2, VSIG4, VWA5A, WDFY4, XCR1, ZBED3-AS1, ZC3H12D, ZCCHC18, ZDHHC22, ZFP36, ZNF185, AATK, ABI3, AC008964.1, AC012593.1, AC109826.1, AIF1, ANKS1B, ANO4, AP001007.1, APOC2, APOL3, ARHGAP25, ARRB2, ASCL2, ASGR2, BATF, C16orf98, C19orf38, C1QA, C1orf162, CAPN9, CARD11, CARTPT, CCDC69, CCL5, CD2, CD27, CD33, CD37, CD3D, CD3E, CHI3L2, CLDND1, CLECL1, CMTM7, CNP, CPVL, CRYBB1, CSF2RA, CTA-384D8.34, CTC-378H22.1, CXCR3, DMBT1, DMRT2, EBI3, EDA, FA2H, FAM214B, FAM78A, FCGR1A, FCGR1B, FCGR1C, FERMT3, FOLH1, FOLR2, FTL, FTLP3, GBGT1, GIMAP1, GIMAP5, GNGT2, GPAT2, GPSM3, GYPC, GZMA, GZMH, GZMK, HAMP, HCN2, HCST, HLA-DPB1, HLA-DPB2, HOXD1, IER3, IL18, KIF19, KLRB1, LARP6, LGALS9, LILRA2, LINC00892, LINC00940, LY86, NANOS1, NCF1, NCF1B, NCF1C, NCF4, NINJ1, NIPA1, NKG7, OR52K3P, OTUD7A, PCED1B-AS1, PILRA, PLA2G2D, PRR15, PRR18, PTCRA, PTPLAD2, RAB40B, RASAL3, RETN, RGS10, RHOG, RNASET2, RNF130, RP11-134L10.1, RP11-214K3.24, RP11-244019.1, RP11-344B5.2, RP11-368J21.3, RP11-436F9.1, RP11-446E9.2, RP11-452C13.1, RP11-483P21.2, RP11-556E13.1, RP11-809C18.4, RP11-8L8.2, RP11-94L15.2, RP11-9G1.3, RP4-719C8.1, RP5-1091N2.9, RP5-899E9.1, RPGRIP1, SELPLG, SH2D1A, SIGLECS, SIRPG, SIT1, SLC15A3, SOWAHD, SPI1, SULT1C2, SYNGR2, TBC1D2, TIMD4, TMEM119, TMEM52B, TMEM92, TNFAIP8L2, TPK1, TRAF3IP3, TREM2, TYROBP, UBASH3A, VAMPS, WAS, XCL2, and ZNF683.

In one embodiment, the expression score is determined from the expression status of a subset of genes. In another embodiment, the expression score is determined from the expression status of a subset of 10 genes. In another embodiment, the expression score is determined from the expression status of a subset of 100 genes. In another embodiment, the expression score is determined from the expression status of a subset of 500 genes. In another embodiment, the expression score is determined from the expression status of a subset of 1000 genes.

In another embodiment, the expression score is determined from the expression status of a subset of 5 or more genes. In some embodiments, the expression score is determined from the expression status of a subset of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 genes.

In some embodiments, the second treatment is given in combination with the first treatment. In another embodiment, the second treatment is given alone. In another embodiment, the first treatment and the second treatment is surgery, chemotherapy, radiation, immunotherapy, gene therapy, or a combination thereof. In other embodiments, the first treatment and the second treatment includes, but is not limited to alkylating agents, antimetabolites, anthracyclines, topoisomerase inhibitors, corticosteroids, antibody therapy (including but not limited to a PD1 antibody therapy or a PD-L1 antibody therapy), a gene therapy and/or gene editing systems (including, but not limited to CRISPR-Cas gene editing systems, chimeric antigen receptor (CAR) gene therapy, and gene transfers (i.e. viral transfection and plasmid transformation)).

In another embodiment, the biological sample is selected from the group consisting of blood, plasma, serum, cerebrospinal fluid, urine, saliva, stool, synovial fluid, a solid tissue sample, and a cell culture sample. In another embodiment, the subject is a human

### Diagnostic Kits and Assays for Identifying Progressing Glioblastoma

In another aspect, disclosed herein is a diagnostic kit or assay for identifying a subject with progressing glioblastoma, wherein the kit or assay comprises components comprising one or more detection means for detecting one or more biomarkers selected from the group comprising CENPF, BIRC5, GAS1, ANLN, RRM2, MKI67, SOX11, CCNB1, UBE2C, CQL11A, MELK, AQP9, NGFR, TREM1, AREG, MARCO, IL1R2, CCL20, NOTCH1, CDK6, ENO1, TREM, PDGFA, HELLS, FANCA, DNMT1, IGF2R, BNIP3L, CLEC5A, CDC25C, PIK3R2, SPP1, BCL2L1, CDH11, FBP1, BIRC5, POLD1, IL1R2, BRIP1, P4HA1, ZEB1, TCF3, EZH2, S100A9, or any combinations thereof.

In other embodiments, the diagnostic kit or assay identifies a subject with progressing glioblastoma, wherein the kit or assay comprises components comprising one or more detection means for detecting one or more biomarkers including, but not limited to AC0055922, AC007879.7, ACTA2, ACTG2, ADAM8, ADM, ANGPTL4, ANKRD1, ANPEP, AOC3, APCDD1L, ATF5, ATP2A3, ATP6VOD2, B3GNT7, BCL3, C15orf48, CA9, CCIN, CCR3, CCR7, CD1C, CD300E, CLCF1, CLDN23, CNN2, COL1A1, COL1A2, COL3A1, COL5A1, COL6A3, CSTA, CXCR4, CYR61, DMBX1, DPP4, EGLN3, EMILIN2, FAM46B, FN1, FOSL1, FOSL2, FPR2, GALNT5, GPR18, HBEGF, HOXB6, HOXB7, ICAM1, IL1R1, IL7R, IL8, IRF1, ITK, KLF4, LCK, LIF, LOX, LUCAT1, MAFF, MAPK13, MEDAG, MEFV, MMP11, MMP9, NABP1, NAMPT, NDRG1, NFKB1A, NLRP12, P4HA3, PAX8, PDLIM1, PIM1, PLAUR, PLEKHS1, PPP1R15A, PTGER2, RAB43, RAC2, RGS1, RGS2. RHOH, RND3, ROR2, RP11-1149023.2, RP11-1149023.3, RP11-290F20.3, RP11-59D5_B.2, RP5-1021120.2, S100A8, S100A9, SDC1, SERPINE1, SLC16A3, SNAIL, SPOCD1, SUSD2, THBD, THBS1, TMEM217, TNFAIP6, TNFAIP8, TNFRSF10D, TREM1, TREML2, TREML3P, VDR, C3H12A, ABCA6, AC002059.10, AC005932.1, AC008060.7, AC011558.5, AC017076.5, AC098823.3, ACSM5, ACTA2-AS1, ADRA2B, APLNR, ARHGEF5, ARRDC2, BEST1, BHLHE41, CD55, CD83, CDK18, CLCA2, CLMN, COL4A5, CPXM2, CTC-1337H24.1, CTD-2034121.2, CXorf65, CYB5R2, DNM1P47, DYSF, EHF, ELOVL1, EVI2A, FAM124A, FAM53B, FGF20, FKBP11, FMN1, GALNT6, GPR37, GTSF1, HMSD, HOXD-AS1, HPN-AS1, ICOSLG, IL9R, IP6K3, KANK4, KCNJ2-AS1, KCNN4, KEL, LAG3, LHB, LINC01010, LRMP, LRP2, LRRC63, LURAP1, MFSD6L, MGST1, MIR24-2, MS4A4E, NFKBID, NME8, OSTF1, P2RX1, PATL2, PCP2, PCSK6, PDLIM7, PLA2G7, PLD1, PM20D1, PPM1N, PRG4, RP11-1094M14.8, RP11-136K7.2, RP11-161H23.5, RP11-16K12.1, RP11-350G8.5, RP11-399019.9, RP11-434H6.6, RP11-597D13.7, RP11-700H6.1, RP11-703116.1, RP11-883G14.1, RP3-47704.14, RP5-943J3.1, RP6-99M1.2, RPS10P7, SAMD3, SEPP1, SH2D3A, SLC45A3, SLC52A1, ST18, SVIP, TM6SF1, TMC7, TMEM139, TMPRSS13, TPM2, TRIM59, TRPC2, TRPM6, TRPV5, U91328.20, UCN2, ABCG1, ABI3BP, AC003003.5, AC004988.1, AC005082.12, AC006129.1, AC006129.2, AC0118999, AC026202.3, ACAT2, ACP5, ACSL1, ADAM28, ADAMTS1, ADAMTS12, ADAP2, ADRB2, AF127936.5, AKR1B10, AKR1B15, ALOX12B, ALOX5, ALOX5AP, AMICA1, AMPD3, ANKRD22, AOAH, AP000476.1, AP000783.1, APBB1IP, APH1B, APOBEC3C, APOBR, ARHGAP30, ARHGDIB, ARHGEF34P, ARHGEF35, ARL11, ARL3, ARRDC4, ASB12, ATP8B4, AXL, B4GALT1, BCL2A1, BCR, BIN2, BIRC3, BMP2K, BTK, C10orf10, C11orf21, C11orf45, C16orf54, C16orf92, C19orf35, C1QC, C2, C3AR1, C4BPB, C5AR1, C5AR2, C9orf47, C9orf66, CABP7, CACNA2D4, CASP1, CASP10, CASP5, CASS4, CBFA2T3, CCL18, CCL2, CCL20, CCL22, CCL8, CCR1, CCR2, CCR4, CCR5, CCRL2, CD14, CD163, CD180, CD1D, CD200R1, CD274, CD28, CD300A, CD300C, CD300LB, CD300LF, CD3G, CD4, CD40, CD40LG, CD53, CD59, CD68, CD69, CD80, CD84, CD86, CD96, CDCP1, CDKN1A, CEACAM21, CEACAM4, CEBPA, CECR1, CELSR3, CFH, CFLAR, CHCHD7, CHRM3-AS2, CIITA, CLEC10A, CLEC12A, CLEC12B, CLEC2B, CLEC7A, CLSTN3, CMAHP, CMKLR1, CMPK2, CNN1, COMP, COQ3, CP, CPA6, CPM, CR1, CREG1, CRTAM, CSF1R, CSF2RB, CSF3R, CTC-205M6.5, CTC-250114.6, CTD-2258A20.5, CTD-2306M10.1, CTD-2540B15.7, CTD-2587H19.1, CTSB, CTSC, CTSH, CTSL, CTSS, CTSZ, CXCL12, CXCL16, CXCL6, CXCR2P1, CXCR6, CXorf21, CYBB, CYP19A1, CYTH4, CYTIP, DAB2, DAPP1, DCN, DCSTAMP, DDO, DDX58, DEC1, DENND3, DLGAP1-AS3, DLX2, DLX6, DLX6-AS1, DMRTC1B, DNAJC5B, DNASE2B, DOCK2, DOCK8, DOK3, DPY19L2P4, DRAM1, DSC2, DUSP1, EFCAB4B, ELL2, EMB, EMR1, EMR2, EMR4P, EPSTI1, EVI2B, F13A1, FAM105A FAM177B, FAM20A, FAM213A, FAM57B, FASLG, FAXC, FBP1, FBXO32, FCER1A, FCER1G, FCGBP, FCGR2A, FCGR2B, FCGR3A, FCRL6, FFAR4, FGD2, FGL2, FGR, FH, FLVCR2, FMO2, FMO4, FMOD, FOXD4, FPR1, FPR3, FSD1, FUCA1, FUT9, GAB3, GADD45B, GAL3ST4, GALNT12, GAPT, GATA3, GBP1P1, GBP2, GBP3, GCH1, GCNT1, GDAP1L1, GDI1, GIMAP2, GIMAP4, GIMAP6, GIMAP7, GIMAP8, GLIPR1, GLRX, GM2A, GNA15, GPNMB, GPR132, GPR141, GPR160, GPR183, GPR34, GPR65, GPR82, GPR84, GPR85, GPRIN3, GRM8, GSK3A, GVINP1, GVQW1, HAVCR2, HCK, HCLS1, HCN4, HLA-DMA, HLA-DMB, HLA-DOA, HLA-DPA1, HLA-DQA2, HLA-DRA, HNMT, HPGDS, HPSE, HTR2B, HTRA4, IFI30, IFI44L, IFIH1, IFIT3, IGF1, IGSF6, IKBKE, IKZF1, IL10, IL10RA, IL12RB1, IL15RA, IL16, IL17RA, IL18BP, IL1A, IL1B, IL1RN, IL21R, IL2RA, IL2RB, IL2RG, IL4I1, IL4R, IL6, ILDR1, IRAK3, IRF4, IRF5, IRF8, IRF9, ITGAL, ITGAM, ITGAX, ITGB2, JPH2, KCNF1, KCNJ5, KCNK13, KCNMB1, KCNQ1, KLC2, KLHDC7B, KLHL6, KLRC1, KLRD1, KMO, KYNU, LA16c-380H5.5, LACTB, LAIR1, LAMP1, LANCL2, LAPTMS, LAT2, LAYN, LBP, LCP1, LCP2, LGALS17A, LILRA1, LILRA6, LILRB1, LILRB2, LILRB4, LILRB5, LINC00539, LINC00578, LINC00702, LINC00968, LPAR5, LPCAT2, LPXN, LRRC25, LRRC39, LRRTM1, LTBP2, LTBR, LUM, LURAP1L, LY9, LYN, LYSMD2, LYVE1, LYZ, M6PR, MAF, MAFB, MALL, MAN1A1, MAN1C1, MAOB, MAP3K8, MCOLN2, MERTK, MGAT4A, MGAT5B, MITF, MKNK1, MLKL, MLPH, MMD2, MNDA, MPEG1, MRC1, MRC1L1, MS4A4A, MS4A6A, MS4A7, MSR1, MX1, MX2, MYO1F, NAAA, NCF2, NCKAP1L, NDUFA6-AS1, NFAM1, NFATC2, NGFR, NGFRAP1. NLRC4, NPL, NRROS, NUDT16P, OAS1, OAS2, OLFML2B, OLFML3, OLR1, OSM, OTUD1, P2RY13, P2RY6, P2RY8, PALD1, PALMD, PAPPA, PARP14, PARP15, PCED1B, PDCD1LG2, PDZD4, PGA5, PIK3AP1, PIK3CD, PIK3R5, PIRT, PJA1, PLA2G15, PLB1, PLBD1, PLCG2, PLEK, PLEKHA3P1, PLEKHO2, PLXDC2, POU2F2, PPARG, PPP1R14C, PPP1R17, PRDM12, PRKCD, PRLR, PRRT4, PTAFR, PTCHD2, PTGDR, PTGER4, PTGFR, PTPN22, PTPN7, PTPRC, PTX3, PYHIN1, RAB20, RAB38, RAB44, RAD51B, RARRES1, RASGRP4, RASL11B, RBM47, RERGL, RGS18, RHBDF2, RHOBTB2, RIN3. RIPK3, RNASE2, RNASE3, RNF112, RNF144B, RP11-1024P17.1, RP11-10K16.1, RP11-1149M10.2, RP11-117D22.2, RP11-119F19.2, RP11-121A8.1, RP11-153M7.1, RP11-153M7.3, RP11-196G18.3, RP11-23P13.6, RP11-245J24.1, RP11-271K11.5, RP11-307C19.1, RP11-325F22.2, RP11-346C20.4, RP11-353N4.5, RP11-357K6.1, RP11-363E6.3, RP11-364B14.3, RP11-379B8.1, RP11-384L8.1, RP11-3B12.1, RP11-403P17.5, RP11-439A17.9, RP11-477J21.6, RP11-47122.3, RP11-497H16.7, RP11-523018.5, RP11-565J7.1, RP11-588H23.3, RP11-626G11.4, RP11-676J15.1, RP11-70C1.1, RP11-733018.1, RP11-750H9.5, RP11-760N9.1, RP11-792D21.2, RP11-838N2.4, RP11-84C10.2, RP11-861A13.4, RP11-90P13.1, RP11-93B14.5, RP11-960L18.1, RP11-986E7.7, RP4-555D20.4, RP4-607I7.1, RPS-1043L.13.1, RPS-1070A16.1, RPS-998N21.4, RPS6KA1, RSAD2, RUSC2, S100Z, SAMSN1, SASH3, SCG3, SCIMP, SCIN, SELE, SERPINA1, SERPINA3, SERPINA5, SERPINB1, SEZ6, SGMS2, SHANK3, SHH, SHISA6, SIGLEC1, SIGLEC10, SIGLEC11, SIGLEC17P, SIGLEC7, SIGLEC9, SIRPB2, SLA, SLAMF1, SLAMF6, SLAMF7, SLAMF8, SLC11A1, SLC16A5, SLC25A22, SLC2A5, SLC2A9, SLC46A2, SLC46A3, SLC7A7, SLC9A9, SLCO2B1, SLFN12L, SMPD3, SMPDL3A, SNAP25-AS1, SNX20, SP110, SP140, SPATA12, SPATC1, SPINT1, SPN, SPP1, SPTBN2, SQRDL, SRRM3, SSTR5, ST14, ST6GAL1, ST8SIA4, STAP1, STARD5, STAT5A, STEAP1B, STEAP4, STX11, STX1B, SULF1, SUSD1, SUSD4, SYK, SYT3, SYTL3, TACSTD2, TAGAP, TAGLN, TAGLN3, TBC1D2B, TBX19, TEC, TERC, TFEC, TGFBR2, TGM2, THBS2, THEMIS, THEMIS2, TIFAB, TLR1, TLR10, TLR2, TLR4, TLR5, TLR6, TLR7, TLR8, TMBIM1, TMC8, TMEM106A, TMEM110, TMEM140, TMEM150B, TMEM173, TMEM176B, TMEM86A, TNFRSF1B, TNFSF13, TNFSF13B, TNFSF15, TNFSF8, TPTE2P1, TRAPPC3L, TRIM22, TRIM38, TRNP1, TRPV4, TSLP, TTC7A, UCP2, USP43, VAMP3, VEGFC, VNN1, VNN2, VSIG4, VWA5A, WDFY4, XCR1, ZBED3-AS1, ZC3H12D, ZCCHC18, ZDHHC22, ZFP36, ZNF185, AATK, ABI3, AC008964.1, AC012593.1, AC109826.1, AIF1, ANKS1B, ANO4, AP001007.1, APOC2, APOL3, ARHGAP25, ARRB2, ASCL2, ASGR2, BATF, C16orf98, C19orf38, C1QA, C1orf162, CAPN9, CARD11, CARTPT, CCDC69, CCL5, CD2, CD27, CD33, CD37, CD3D, CD3E, CHI3L2, CLDND1, CLECL1, CMTM7, CNP, CPVL, CRYBB1, CSF2RA, CTA-384D8.34, CTC-378H22.1, CXCR3, DMBT1, DMRT2, EBI3, EDA, FA2H, FAM214B, FAM78A, FCGR1A, FCGR1B, FCGR1C, FERMT3, FOLH1, FOLR2, FTL, FTLP3, GBGT1, GIMAP1, GIMAP5, GNGT2, GPAT2, GPSM3, GYPC, GZMA, GZMH, GZMK, HAMP, HCN2, HCST, HLA-DPB1, HLA-DPB2, HOXD1, IER3, IL18, KIF19, KLRB1, LARP6, LGALS9, LILRA2, LINC00892, LINC00940, LY86, NANOS1, NCF1, NCF1B, NCF1C, NCF4, NINJ1, NIPA1, NKG7, OR52K3P. OTUD7A, PCED1B-AS1, PILRA, PLA2G2D, PRR15, PRR18, PTCRA, PTPLAD2, RAB40B, RASAL3, RETN, RGS10, RHOG, RNASET2, RNF130, RP11-134L10.1, RP11-214K3.24, RP11-244019.1, RP11-344B5.2, RP11-368J21.3, RP11-436F9.1, RP11-446E9.2, RP11-452C13.1, RP11-483P21.2, RP11-556E13.1, RP11-809C18.4, RP11-8L8.2, RP11-94L15.2, RP11-9G1.3, RP4-719C8.1, RP5-1091N2.9, RP5-899E9.1, RPGRIP1, SELPLG, SH2D1A, SIGLECS, SIRPG, SIT1, SLC15A3, SOWAHD, SPI1, SULT1C2, SYNGR2, TBC1D2, TIMD4, TMEM119, TMEM52B, TMEM92, TNFAIP8L2, TPK1, TRAF3IP3, TREM2, TYROBP, UBASH3A, VAMPS, WAS, XCL2, and ZNF683.

In one embodiment, said detection means comprise enzyme linked immunosorbent assay (ELISA), immunofluorescence, western blotting, immunodiffusion, polymerase chain reaction (PCR), or flow cytometry. In other embodiments, said detection means further comprises one or more reagents for determining the presence of one or more biomarkers selected from 1000 or more genes.

In some embodiments, said detection means further comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more reagents for determining the presence of one or more biomarkers selected from 1000 or more genes.

In some embodiments, one or more biomarkers is selected from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000 or more biomarkers are selected from 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 1751, 1752, 1753, 1754, 1755, 1756, 1757, 1758, 1759, 1760, 1761, 1762, 1763, 1764, 1765, 1766, 1767, 1768, 1769, 1770, 1771, 1772, 1773, 1774, 1775, 1776, 1777, 1778, 1779, 1780, 1781, 1782, 1783, 1784, 1785, 1786, 1787, 1788, 1789, 1790, 1791, 1792, 1793, 1794, 1795, 1796, 1797, 1798, 1799, 1800, 1801, 1802, 1803, 1804, 1805, 1806, 1807, 1808, 1809, 1810, 1811, 1812, 1813, 1814, 1815, 1816, 1817, 1818, 1819, 1820, 1821, 1822, 1823, 1824, 1825, 1826, 1827, 1828, 1829, 1830, 1831, 1832, 1833, 1834, 1835, 1836, 1837, 1838, 1839, 1840, 1841, 1842, 1843, 1844, 1845, 1846, 1847, 1848, 1849, 1850, 1851, 1852, 1853, 1854, 1855, 1856, 1857, 1858, 1859, 1860, 1861, 1862, 1863, 1864, 1865, 1866, 1867, 1868, 1869, 1870, 1871, 1872, 1873, 1874, 1875, 1876, 1877, 1878, 1879, 1880, 1881, 1882, 1883, 1884, 1885, 1886, 1887, 1888, 1889, 1890, 1891, 1892, 1893, 1894, 1895, 1896, 1897, 1898, 1899, 1900, 1901, 1902, 1903, 1904, 1905, 1906, 1907, 1908, 1909, 1910, 1911, 1912, 1913, 1914, 1915, 1916, 1917, 1918, 1919, 1920, 1921, 1922, 1923, 1924, 1925, 1926, 1927, 1928, 1929, 1930, 1931, 1932, 1933, 1934, 1935, 1936, 1937, 1938, 1939, 1940, 1941, 1942, 1943, 1944, 1945, 1946, 1947, 1948, 1949, 1950, 1951, 1952, 1953, 1954, 1955, 1956, 1957, 1958, 1959, 1960, 1961, 1962, 1963, 1964, 1965, 1966, 1967, 1968, 1969, 1970, 1971, 1972, 1973, 1974, 1975, 1976, 1977, 1978, 1979, 1980, 1981, 1982, 1983, 1984, 1985, 1986, 1987, 1988, 1989, 1990, 1991, 1992, 1993, 1994, 1995, 1996, 1997, 1998, 1999, 2000, 2001, 2002, 2003, 2004, 2005, 2006, 2007, 2008, 2009, 2010, 2011, 2012, 2013, 2014, 2015, 2016, 2017, 2018, 2019, 2020, 2021, 2022, 2023, 2024, 2025, 2026, 2027, 2028, 2029, 2030, 2031, 2032, 2033, 2034, 2035, 2036, 2037, 2038, 2039, 2040, 2041, 2042, 2043, 2044, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2097, 2098, 2099, 2100, 2101, 2102, 2103, 2104, 2105, 2106, 2107, 2108, 2109, 2110, 2111, 2112, 2113, 2114, 2115, 2116, 2117, 2118, 2119, 2120, 2121, 2122, 2123, 2124, 2125, 2126, 2127, 2128, 2129, 2130, 2131, 2132, 2133, 2134, 2135, 2136, 2137, 2138, 2139, 2140, 2141, 2142, 2143, 2144, 2145, 2146, 2147, 2148, 2149, 2150, 2151, 2152, 2153, 2154, 2155, 2156, 2157, 2158, 2159, 2160, 2161, 2162, 2163, 2164, 2165, 2166, 2167, 2168, 2169, 2170, 2171, 2172, 2173, 2174, 2175, 2176, 2177, 2178, 2179, 2180, 2181, 2182, 2183, 2184, 2185, 2186, 2187, 2188, 2189, 2190, 2191, 2192, 2193, 2194, 2195, 2196, 2197, 2198, 2199, 2200, 2201, 2202, 2203, 2204, 2205, 2206, 2207, 2208, 2209, 2210, 2211, 2212, 2213, 2214, 2215, 2216, 2217, 2218, 2219, 2220, 2221, 2222, 2223, 2224, 2225, 2226, 2227, 2228, 2229, 2230, 2231, 2232, 2233, 2234, 2235, 2236, 2237, 2238, 2239, 2240, 2241, 2242, 2243, 2244, 2245, 2246, 2247, 2248, 2249, 2250, 2251, 2252, 2253, 2254, 2255, 2256, 2257, 2258, 2259, 2260, 2261, 2262, 2263, 2264, 2265, 2266, 2267, 2268, 2269, 2270, 2271, 2272, 2273, 2274, 2275, 2276, 2277, 2278, 2279, 2280, 2281, 2282, 2283, 2284, 2285, 2286, 2287, 2288, 2289, 2290, 2291, 2292, 2293, 2294, 2295, 2296, 2297, 2298, 2299, 2300, 2301, 2302, 2303, 2304, 2305, 2306, 2307, 2308, 2309, 2310, 2311, 2312, 2313, 2314, 2315, 2316, 2317, 2318, 2319, 2320, 2321, 2322, 2323, 2324, 2325, 2326, 2327, 2328, 2329, 2330, 2331, 2332, 2333, 2334, 2335, 2336, 2337, 2338, 2339, 2340, 2341, 2342, 2343, 2344, 2345, 2346, 2347, 2348, 2349, 2350, 2351, 2352, 2353, 2354, 2355, 2356, 2357, 2358, 2359, 2360, 2361, 2362, 2363, 2364, 2365, 2366, 2367, 2368, 2369, 2370, 2371, 2372, 2373, 2374, 2375, 2376, 2377, 2378, 2379, 2380, 2381, 2382, 2383, 2384, 2385, 2386, 2387, 2388, 2389, 2390, 2391, 2392, 2393, 2394, 2395, 2396, 2397, 2398, 2399, 2400, 2401, 2402, 2403, 2404, 2405, 2406, 2407, 2408, 2409, 2410, 2411, 2412, 2413, 2414, 2415, 2416, 2417, 2418, 2419, 2420, 2421, 2422, 2423, 2424, 2425, 2426, 2427, 2428, 2429, 2430, 2431, 2432, 2433, 2434, 2435, 2436, 2437, 2438, 2439, 2440, 2441, 2442, 2443, 2444, 2445, 2446, 2447, 2448, 2449, 2450, 2451, 2452, 2453, 2454, 2455, 2456, 2457, 2458, 2459, 2460, 2461, 2462, 2463, 2464, 2465, 2466, 2467, 2468, 2469, 2470, 2471, 2472, 2473, 2474, 2475, 2476, 2477, 2478, 2479, 2480, 2481, 2482, 2483, 2484, 2485, 2486, 2487, 2488, 2489, 2490, 2491, 2492, 2493, 2494, 2495, 2496, 2497, 2498, 2499, 2500, 2501, 2502, 2503, 2504, 2505, 2506, 2507, 2508, 2509, 2510, 2511, 2512, 2513, 2514, 2515, 2516, 2517, 2518, 2519, 2520, 2521, 2522, 2523, 2524, 2525, 2526, 2527, 2528, 2529, 2530, 2531, 2532, 2533, 2534, 2535, 2536, 2537, 2538, 2539, 2540, 2541, 2542, 2543, 2544, 2545, 2546, 2547, 2548, 2549, 2550, 2551, 2552, 2553, 2554, 2555, 2556, 2557, 2558, 2559, 2560, 2561, 2562, 2563, 2564, 2565, 2566, 2567, 2568, 2569, 2570, 2571, 2572, 2573, 2574, 2575, 2576, 2577, 2578, 2579, 2580, 2581, 2582, 2583, 2584, 2585, 2586, 2587, 2588, 2589, 2590, 2591, 2592, 2593, 2594, 2595, 2596, 2597, 2598, 2599, 2600, 2601, 2602, 2603, 2604, 2605, 2606, 2607, 2608, 2609, 2610, 2611, 2612, 2613, 2614, 2615, 2616, 2617, 2618, 2619, 2620, 2621, 2622, 2623, 2624, 2625, 2626, 2627, 2628, 2629, 2630, 2631, 2632, 2633, 2634, 2635, 2636, 2637, 2638, 2639, 2640, 2641, 2642, 2643, 2644, 2645, 2646, 2647, 2648, 2649, 2650, 2651, 2652, 2653, 2654, 2655, 2656, 2657, 2658, 2659, 2660, 2661, 2662, 2663, 2664, 2665, 2666, 2667, 2668, 2669, 2670, 2671, 2672, 2673, 2674, 2675, 2676, 2677, 2678, 2679, 2680, 2681, 2682, 2683, 2684, 2685, 2686, 2687, 2688, 2689, 2690, 2691, 2692, 2693, 2694, 2695, 2696, 2697, 2698, 2699, 2700, 2701, 2702, 2703, 2704, 2705, 2706, 2707, 2708, 2709, 2710, 2711, 2712, 2713, 2714, 2715, 2716, 2717, 2718, 2719, 2720, 2721, 2722, 2723, 2724, 2725, 2726, 2727, 2728, 2729, 2730, 2731, 2732, 2733, 2734, 2735, 2736, 2737, 2738, 2739, 2740, 2741, 2742, 2743, 2744, 2745, 2746, 2747, 2748, 2749, 2750, 2751, 2752, 2753, 2754, 2755, 2756, 2757, 2758, 2759, 2760, 2761, 2762, 2763, 2764, 2765, 2766, 2767, 2768, 2769, 2770, 2771, 2772, 2773, 2774, 2775, 2776, 2777, 2778, 2779, 2780, 2781, 2782, 2783, 2784, 2785, 2786, 2787, 2788, 2789, 2790, 2791, 2792, 2793, 2794, 2795, 2796, 2797, 2798, 2799, 2800, 2801, 2802, 2803, 2804, 2805, 2806, 2807, 2808, 2809, 2810, 2811, 2812, 2813, 2814, 2815, 2816, 2817, 2818, 2819, 2820, 2821, 2822, 2823, 2824, 2825, 2826, 2827, 2828, 2829, 2830, 2831, 2832, 2833, 2834, 2835, 2836, 2837, 2838, 2839, 2840, 2841, 2842, 2843, 2844, 2845, 2846, 2847, 2848, 2849, 2850, 2851, 2852, 2853, 2854, 2855, 2856, 2857, 2858, 2859, 2860, 2861, 2862, 2863, 2864, 2865, 2866, 2867, 2868, 2869, 2870, 2871, 2872, 2873, 2874, 2875, 2876, 2877, 2878, 2879, 2880, 2881, 2882, 2883, 2884, 2885, 2886, 2887, 2888, 2889, 2890, 2891, 2892, 2893, 2894, 2895, 2896, 2897, 2898, 2899, 2900, 2901, 2902, 2903, 2904, 2905, 2906, 2907, 2908, 2909, 2910, 2911, 2912, 2913, 2914, 2915, 2916, 2917, 2918, 2919, 2920, 2921, 2922, 2923, 2924, 2925, 2926, 2927, 2928, 2929, 2930, 2931, 2932, 2933, 2934, 2935, 2936, 2937, 2938, 2939, 2940, 2941, 2942, 2943, 2944, 2945, 2946, 2947, 2948, 2949, 2950, 2951, 2952, 2953, 2954, 2955, 2956, 2957, 2958, 2959, 2960, 2961, 2962, 2963, 2964, 2965, 2966, 2967, 2968, 2969, 2970, 2971, 2972, 2973, 2974, 2975, 2976, 2977, 2978, 2979, 2980, 2981, 2982, 2983, 2984, 2985, 2986, 2987, 2988, 2989, 2990, 2991, 2992, 2993, 2994, 2995, 2996, 2997, 2998, 2999, 3000, 3001, 3002, 3003, 3004, 3005, 3006, 3007, 3008, 3009, 3010, 3011, 3012, 3013, 3014, 3015, 3016, 3017, 3018, 3019, 3020, 3021, 3022, 3023, 3024, 3025, 3026, 3027, 3028, 3029, 3030, 3031, 3032, 3033, 3034, 3035, 3036, 3037, 3038, 3039, 3040, 3041, 3042, 3043, 3044, 3045, 3046, 3047, 3048, 3049, 3050, 3051, 3052, 3053, 3054, 3055, 3056, 3057, 3058, 3059, 3060, 3061, 3062, 3063, 3064, 3065, 3066, 3067, 3068, 3069, 3070, 3071, 3072, 3073, 3074, 3075, 3076, 3077, 3078, 3079, 3080, 3081, 3082, 3083, 3084, 3085, 3086, 3087, 3088, 3089, 3090, 3091, 3092, 3093, 3094, 3095, 3096, 3097, 3098, 3099, 3100, 3101, 3102, 3103, 3104, 3105, 3106, 3107, 3108, 3109, 3110, 3111, 3112, 3113, 3114, 3115, 3116, 3117, 3118, 3119, 3120, 3121, 3122, 3123, 3124, 3125, 3126, 3127, 3128, 3129, 3130, 3131, 3132, 3133, 3134, 3135, 3136, 3137, 3138, 3139, 3140, 3141, 3142, 3143, 3144, 3145, 3146, 3147, 3148, 3149, 3150, 3151, 3152, 3153, 3154, 3155, 3156, 3157, 3158, 3159, 3160, 3161, 3162, 3163, 3164, 3165, 3166, 3167, 3168, 3169, 3170, 3171, 3172, 3173, 3174, 3175, 3176, 3177, 3178, 3179, 3180, 3181, 3182, 3183, 3184, 3185, 3186, 3187, 3188, 3189, 3190, 3191, 3192, 3193, 3194, 3195, 3196, 3197, 3198, 3199, 3200, 3201, 3202, 3203, 3204, 3205, 3206, 3207, 3208, 3209, 3210, 3211, 3212, 3213, 3214, 3215, 3216, 3217, 3218, 3219, 3220, 3221, 3222, 3223, 3224, 3225, 3226, 3227, 3228, 3229, 3230, 3231, 3232, 3233, 3234, 3235, 3236, 3237, 3238, 3239, 3240, 3241, 3242, 3243, 3244, 3245, 3246, 3247, 3248, 3249, 3250, 3251, 3252, 3253, 3254, 3255, 3256, 3257, 3258, 3259, 3260, 3261, 3262, 3263, 3264, 3265, 3266, 3267, 3268, 3269, 3270, 3271, 3272, 3273, 3274, 3275, 3276, 3277, 3278, 3279, 3280, 3281, 3282, 3283, 3284, 3285, 3286, 3287, 3288, 3289, 3290, 3291, 3292, 3293, 3294, 3295, 3296, 3297, 3298, 3299, 3300, 3301, 3302, 3303, 3304, 3305, 3306, 3307, 3308, 3309, 3310, 3311, 3312, 3313, 3314, 3315, 3316, 3317, 3318, 3319, 3320, 3321, 3322, 3323, 3324, 3325, 3326, 3327, 3328, 3329, 3330, 3331, 3332, 3333, 3334, 3335, 3336, 3337, 3338, 3339, 3340, 3341, 3342, 3343, 3344, 3345, 3346, 3347, 3348, 3349, 3350, 3351, 3352, 3353, 3354, 3355, 3356, 3357, 3358, 3359, 3360, 3361, 3362, 3363, 3364, 3365, 3366, 3367, 3368, 3369, 3370, 3371, 3372, 3373, 3374, 3375, 3376, 3377, 3378, 3379, 3380, 3381, 3382, 3383, 3384, 3385, 3386, 3387, 3388, 3389, 3390, 3391, 3392, 3393, 3394, 3395, 3396, 3397, 3398, 3399, 3400, 3401, 3402, 3403, 3404, 3405, 3406, 3407, 3408, 3409, 3410, 3411, 3412, 3413, 3414, 3415, 3416, 3417, 3418, 3419, 3420, 3421, 3422, 3423, 3424, 3425, 3426, 3427, 3428, 3429, 3430, 3431, 3432, 3433, 3434, 3435, 3436, 3437, 3438, 3439, 3440, 3441, 3442, 3443, 3444, 3445, 3446, 3447, 3448, 3449, 3450, 3451, 3452, 3453, 3454, 3455, 3456, 3457, 3458, 3459, 3460, 3461, 3462, 3463, 3464, 3465, 3466, 3467, 3468, 3469, 3470, 3471, 3472, 3473, 3474, 3475, 3476, 3477, 3478, 3479, 3480, 3481, 3482, 3483, 3484, 3485, 3486, 3487, 3488, 3489, 3490, 3491, 3492, 3493, 3494, 3495, 3496, 3497, 3498, 3499, 3500, 3501, 3502, 3503, 3504, 3505, 3506, 3507, 3508, 3509, 3510, 3511, 3512, 3513, 3514, 3515, 3516, 3517, 3518, 3519, 3520, 3521, 3522, 3523, 3524, 3525, 3526, 3527, 3528, 3529, 3530, 3531, 3532, 3533, 3534, 3535, 3536, 3537, 3538, 3539, 3540, 3541, 3542, 3543, 3544, 3545, 3546, 3547, 3548, 3549, 3550, 3551, 3552, 3553, 3554, 3555, 3556, 3557, 3558, 3559, 3560, 3561, 3562, 3563, 3564, 3565, 3566, 3567, 3568, 3569, 3570, 3571, 3572, 3573, 3574, 3575, 3576, 3577, 3578, 3579, 3580, 3581, 3582, 3583, 3584, 3585, 3586, 3587, 3588, 3589, 3590, 3591, 3592, 3593, 3594, 3595, 3596, 3597, 3598, 3599, 3600, 3601, 3602, 3603, 3604, 3605, 3606, 3607, 3608, 3609, 3610, 3611, 3612, 3613, 3614, 3615, 3616, 3617, 3618, 3619, 3620, 3621, 3622, 3623, 3624, 3625, 3626, 3627, 3628, 3629, 3630, 3631, 3632, 3633, 3634, 3635, 3636, 3637, 3638, 3639, 3640, 3641, 3642, 3643, 3644, 3645, 3646, 3647, 3648, 3649, 3650, 3651, 3652, 3653, 3654, 3655, 3656, 3657, 3658, 3659, 3660, 3661, 3662, 3663, 3664, 3665, 3666, 3667, 3668, 3669, 3670, 3671, 3672, 3673, 3674, 3675, 3676, 3677, 3678, 3679, 3680, 3681, 3682, 3683, 3684, 3685, 3686, 3687, 3688, 3689, 3690, 3691, 3692, 3693, 3694, 3695, 3696, 3697, 3698, 3699, 3700, 3701, 3702, 3703, 3704, 3705, 3706, 3707, 3708, 3709, 3710, 3711, 3712, 3713, 3714, 3715, 3716, 3717, 3718, 3719, 3720, 3721, 3722, 3723, 3724, 3725, 3726, 3727, 3728, 3729, 3730, 3731, 3732, 3733, 3734, 3735, 3736, 3737, 3738, 3739, 3740, 3741, 3742, 3743, 3744, 3745, 3746, 3747, 3748, 3749, 3750, 3751, 3752, 3753, 3754, 3755, 3756, 3757, 3758, 3759, 3760, 3761, 3762, 3763, 3764, 3765, 3766, 3767, 3768, 3769, 3770, 3771, 3772, 3773, 3774, 3775, 3776, 3777, 3778, 3779, 3780, 3781, 3782, 3783, 3784, 3785, 3786, 3787, 3788, 3789, 3790, 3791, 3792, 3793, 3794, 3795, 3796, 3797, 3798, 3799, 3800, 3801, 3802, 3803, 3804, 3805, 3806, 3807, 3808, 3809, 3810, 3811, 3812, 3813, 3814, 3815, 3816, 3817, 3818, 3819, 3820, 3821, 3822, 3823, 3824, 3825, 3826, 3827, 3828, 3829, 3830, 3831, 3832, 3833, 3834, 3835, 3836, 3837, 3838, 3839, 3840, 3841, 3842, 3843, 3844, 3845, 3846, 3847, 3848, 3849, 3850, 3851, 3852, 3853, 3854, 3855, 3856, 3857, 3858, 3859, 3860, 3861, 3862, 3863, 3864, 3865, 3866, 3867, 3868, 3869, 3870, 3871, 3872, 3873, 3874, 3875, 3876, 3877, 3878, 3879, 3880, 3881, 3882, 3883, 3884, 3885, 3886, 3887, 3888, 3889, 3890, 3891, 3892, 3893, 3894, 3895, 3896, 3897, 3898, 3899, 3900, 3901, 3902, 3903, 3904, 3905, 3906, 3907, 3908, 3909, 3910, 3911, 3912, 3913, 3914, 3915, 3916, 3917, 3918, 3919, 3920, 3921, 3922, 3923, 3924, 3925, 3926, 3927, 3928, 3929, 3930, 3931, 3932, 3933, 3934, 3935, 3936, 3937, 3938, 3939, 3940, 3941, 3942, 3943, 3944, 3945, 3946, 3947, 3948, 3949, 3950, 3951, 3952, 3953, 3954, 3955, 3956, 3957, 3958, 3959, 3960, 3961, 3962, 3963, 3964, 3965, 3966, 3967, 3968, 3969, 3970, 3971, 3972, 3973, 3974, 3975, 3976, 3977, 3978, 3979, 3980, 3981, 3982, 3983, 3984, 3985, 3986, 3987, 3988, 3989, 3990, 3991, 3992, 3993, 3994, 3995, 3996, 3997, 3998, 3999, 4000, 4001, 4002, 4003, 4004, 4005, 4006, 4007, 4008, 4009, 4010, 4011, 4012, 4013, 4014, 4015, 4016, 4017, 4018, 4019, 4020, 4021, 4022, 4023, 4024, 4025, 4026, 4027, 4028, 4029, 4030, 4031, 4032, 4033, 4034, 4035, 4036, 4037, 4038, 4039, 4040, 4041, 4042, 4043, 4044, 4045, 4046, 4047, 4048, 4049, 4050, 4051, 4052, 4053, 4054, 4055, 4056, 4057, 4058, 4059, 4060, 4061, 4062, 4063, 4064, 4065, 4066, 4067, 4068, 4069, 4070, 4071, 4072, 4073, 4074, 4075, 4076, 4077, 4078, 4079, 4080, 4081, 4082, 4083, 4084, 4085, 4086, 4087, 4088, 4089, 4090, 4091, 4092, 4093, 4094, 4095, 4096, 4097, 4098, 4099, 4100, 4101, 4102, 4103, 4104, 4105, 4106, 4107, 4108, 4109, 4110, 4111, 4112, 4113, 4114, 4115, 4116, 4117, 4118, 4119, 4120, 4121, 4122, 4123, 4124, 4125, 4126, 4127, 4128, 4129, 4130, 4131, 4132, 4133, 4134, 4135, 4136, 4137, 4138, 4139, 4140, 4141, 4142, 4143, 4144, 4145, 4146, 4147, 4148, 4149, 4150, 4151, 4152, 4153, 4154, 4155, 4156, 4157, 4158, 4159, 4160, 4161, 4162, 4163, 4164, 4165, 4166, 4167, 4168, 4169, 4170, 4171, 4172, 4173, 4174, 4175, 4176, 4177, 4178, 4179, 4180, 4181, 4182, 4183, 4184, 4185, 4186, 4187, 4188, 4189, 4190, 4191, 4192, 4193, 4194, 4195, 4196, 4197, 4198, 4199, 4200, 4201, 4202, 4203, 4204, 4205, 4206, 4207, 4208, 4209, 4210, 4211, 4212, 4213, 4214, 4215, 4216, 4217, 4218, 4219, 4220, 4221, 4222, 4223, 4224, 4225, 4226, 4227, 4228, 4229, 4230, 4231, 4232, 4233, 4234, 4235, 4236, 4237, 4238, 4239, 4240, 4241, 4242, 4243, 4244, 4245, 4246, 4247, 4248, 4249, 4250, 4251, 4252, 4253, 4254, 4255, 4256, 4257, 4258, 4259, 4260, 4261, 4262, 4263, 4264, 4265, 4266, 4267, 4268, 4269, 4270, 4271, 4272, 4273, 4274, 4275, 4276, 4277, 4278, 4279, 4280, 4281, 4282, 4283, 4284, 4285, 4286, 4287, 4288, 4289, 4290, 4291, 4292, 4293, 4294, 4295, 4296, 4297, 4298, 4299, 4300, 4301, 4302, 4303, 4304, 4305, 4306, 4307, 4308, 4309, 4310, 4311, 4312, 4313, 4314, 4315, 4316, 4317, 4318, 4319, 4320, 4321, 4322, 4323, 4324, 4325, 4326, 4327, 4328, 4329, 4330, 4331, 4332, 4333, 4334, 4335, 4336, 4337, 4338, 4339, 4340, 4341, 4342, 4343, 4344, 4345, 4346, 4347, 4348, 4349, 4350, 4351, 4352, 4353, 4354, 4355, 4356, 4357, 4358, 4359, 4360, 4361, 4362, 4363, 4364, 4365, 4366, 4367, 4368, 4369, 4370, 4371, 4372, 4373, 4374, 4375, 4376, 4377, 4378, 4379, 4380, 4381, 4382, 4383, 4384, 4385, 4386, 4387, 4388, 4389, 4390, 4391, 4392, 4393, 4394, 4395, 4396, 4397, 4398, 4399, 4400, 4401, 4402, 4403, 4404, 4405, 4406, 4407, 4408, 4409, 4410, 4411, 4412, 4413, 4414, 4415, 4416, 4417, 4418, 4419, 4420, 4421, 4422, 4423, 4424, 4425, 4426, 4427, 4428, 4429, 4430, 4431, 4432, 4433, 4434, 4435, 4436, 4437, 4438, 4439, 4440, 4441, 4442, 4443, 4444, 4445, 4446, 4447, 4448, 4449, 4450, 4451, 4452, 4453, 4454, 4455, 4456, 4457, 4458, 4459, 4460, 4461, 4462, 4463, 4464, 4465, 4466, 4467, 4468, 4469, 4470, 4471, 4472, 4473, 4474, 4475, 4476, 4477, 4478, 4479, 4480, 4481, 4482, 4483, 4484, 4485, 4486, 4487, 4488, 4489, 4490, 4491, 4492, 4493, 4494, 4495, 4496, 4497, 4498, 4499, 4500, 4501, 4502, 4503, 4504, 4505, 4506, 4507, 4508, 4509, 4510, 4511, 4512, 4513, 4514, 4515, 4516, 4517, 4518, 4519, 4520, 4521, 4522, 4523, 4524, 4525, 4526, 4527, 4528, 4529, 4530, 4531, 4532, 4533, 4534, 4535, 4536, 4537, 4538, 4539, 4540, 4541, 4542, 4543, 4544, 4545, 4546, 4547, 4548, 4549, 4550, 4551, 4552, 4553, 4554, 4555, 4556, 4557, 4558, 4559, 4560, 4561, 4562, 4563, 4564, 4565, 4566, 4567, 4568, 4569, 4570, 4571, 4572, 4573, 4574, 4575, 4576, 4577, 4578, 4579, 4580, 4581, 4582, 4583, 4584, 4585, 4586, 4587, 4588, 4589, 4590, 4591, 4592, 4593, 4594, 4595, 4596, 4597, 4598, 4599, 4600, 4601, 4602, 4603, 4604, 4605, 4606, 4607, 4608, 4609, 4610, 4611, 4612, 4613, 4614, 4615, 4616, 4617, 4618, 4619, 4620, 4621, 4622, 4623, 4624, 4625, 4626, 4627, 4628, 4629, 4630, 4631, 4632, 4633, 4634, 4635, 4636, 4637, 4638, 4639, 4640, 4641, 4642, 4643, 4644, 4645, 4646, 4647, 4648, 4649, 4650, 4651, 4652, 4653, 4654, 4655, 4656, 4657, 4658, 4659, 4660, 4661, 4662, 4663, 4664, 4665, 4666, 4667, 4668, 4669, 4670, 4671, 4672, 4673, 4674, 4675, 4676, 4677, 4678, 4679, 4680, 4681, 4682, 4683, 4684, 4685, 4686, 4687, 4688, 4689, 4690, 4691, 4692, 4693, 4694, 4695, 4696, 4697, 4698, 4699, 4700, 4701, 4702, 4703, 4704, 4705, 4706, 4707, 4708, 4709, 4710, 4711, 4712, 4713, 4714, 4715, 4716, 4717, 4718, 4719, 4720, 4721, 4722, 4723, 4724, 4725, 4726, 4727, 4728, 4729, 4730, 4731, 4732, 4733, 4734, 4735, 4736, 4737, 4738, 4739, 4740, 4741, 4742, 4743, 4744, 4745, 4746, 4747, 4748, 4749, 4750, 4751, 4752, 4753, 4754, 4755, 4756, 4757, 4758, 4759, 4760, 4761, 4762, 4763, 4764, 4765, 4766, 4767, 4768, 4769, 4770, 4771, 4772, 4773, 4774, 4775, 4776, 4777, 4778, 4779, 4780, 4781, 4782, 4783, 4784, 4785, 4786, 4787, 4788, 4789, 4790, 4791, 4792, 4793, 4794, 4795, 4796, 4797, 4798, 4799, 4800, 4801, 4802, 4803, 4804, 4805, 4806, 4807, 4808, 4809, 4810, 4811, 4812, 4813, 4814, 4815, 4816, 4817, 4818, 4819, 4820, 4821, 4822, 4823, 4824, 4825, 4826, 4827, 4828, 4829, 4830, 4831, 4832, 4833, 4834, 4835, 4836, 4837, 4838, 4839, 4840, 4841, 4842, 4843, 4844, 4845, 4846, 4847, 4848, 4849, 4850, 4851, 4852, 4853, 4854, 4855, 4856, 4857, 4858, 4859, 4860, 4861, 4862, 4863, 4864, 4865, 4866, 4867, 4868, 4869, 4870, 4871, 4872, 4873, 4874, 4875, 4876, 4877, 4878, 4879, 4880, 4881, 4882, 4883, 4884, 4885, 4886, 4887, 4888, 4889, 4890, 4891, 4892, 4893, 4894, 4895, 4896, 4897, 4898, 4899, 4900, 4901, 4902, 4903, 4904, 4905, 4906, 4907, 4908, 4909, 4910, 4911, 4912, 4913, 4914, 4915, 4916, 4917, 4918, 4919, 4920, 4921, 4922, 4923, 4924, 4925, 4926, 4927, 4928, 4929, 4930, 4931, 4932, 4933, 4934, 4935, 4936, 4937, 4938, 4939, 4940, 4941, 4942, 4943, 4944, 4945, 4946, 4947, 4948, 4949, 4950, 4951, 4952, 4953, 4954, 4955, 4956, 4957, 4958, 4959, 4960, 4961, 4962, 4963, 4964, 4965, 4966, 4967, 4968, 4969, 4970, 4971, 4972, 4973, 4974, 4975, 4976, 4977, 4978, 4979, 4980, 4981, 4982, 4983, 4984, 4985, 4986, 4987, 4988, 4989, 4990, 4991, 4992, 4993, 4994, 4995, 4996, 4997, 4998, 4999, 5000, or more genes.

In another embodiment, the one or more biomarkers is selected from a subset of genes. In another embodiment, the one or more biomarkers is selected from a subset of 10 genes. In another embodiment, the one or more biomarkers is selected from a subset of 100 genes. In another embodiment, the one or more biomarkers is selected from a subset of 500 genes. In another embodiment, the one or more biomarkers is selected from a subset of 1000 genes.

In another embodiment, the one or more biomarkers is selected from a subset of 5 or more genes. In some embodiments, the one or more biomarkers is selected from a subset of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 genes.

In some embodiments, said reagent is a monoclonal antibody or any antigen binding fragment thereof coupled to a detectable reagent. In some embodiments, the detectable reagent comprises an enzyme, a fluorescent label, a radiolabel, or a chemiluminescent label. In some embodiments, the detectable reagent includes, but is not limited to luciferase, green fluorescent protein (GFP), yellow fluorescent protein (YFP), blue fluorescent protein (BFP), cyane fluorescent protein (CFP), monomeric red fluorescent protein (mRFP), Discosoma striata (DsRed), mCherry, mOrange, tdTomato, mSTrawberry, mPlum, photoactivatable GFP (PA-GFP), Venus, Kaede, monomeric kusabira orange (mKO), Dronpa, enhanced CFP (ECFP), Emerald, Cyan fluorescent protein for energy transfer (CyPet), super CFP (SCFP), Cerulean, photoswitchable CFP (PS-CFP2), photoactivatable RFP1 (PA-RFP1), photoactivatable mCherry (PA-mCherry), monomeric teal fluorescent protein (mTFP1), Eos fluorescent protein (EosFP), Dendra, TagBFP, TagRFP, enhanced YFP (EYFP), Topaz, Citrine, yellow fluorescent protein for energy transfer (YPet), super YFP (SYFP), enhanced GFP (EGFP), Superfolder GFP, T-Sapphire, Fucci, mKO2, mOrange2, mApple, Sirius, Azurite, EBFP, EBFP2, Alexa Fluor 350, Alexa Fluor 405, Alexa Fluor 488, Alexa Fluor 532, Alexa Fluor 546, Alexa Fluor 555, Alexa Fluor 561, Alexa Fluor 568, Alexa Fluor 594, Alexa Fluor 647, Alexa Fluor 660, Alexa Fluor 680, Alexa Fluor 700, Alexa Fluor 750, BODIPY FL, Courmarin, Cy3, Cy5, Fluorescein (FITC), Oregon Green, Pacific Blue, Pacific Green, Pacific Orange, PE-Cyanine7, PerCP-Cyanine5.5, Tetramethylrhodamine (TRITC), Texas Red, DAPI, Propidium Iodide, SYTO 9, SYTOX Green, TO-PRO-3, Allophycocyanin (APC), R-Phycoerythrin (R-PE), Cyan Fluorescent Protein (CFP), Green Fluorescent Protein (GFP), Red Fluorescent Protein (RFP), and Blue Fluorescent Protein (BFP).

In other embodiments, said biomarkers are detected in a biological sample from the subject. In another embodiment, the biological sample is selected from the group consisting of blood, plasma, serum, cerebrospinal fluid, urine, saliva, stool, synovial fluid, a solid tissue sample, and a cell culture sample. In another embodiment, the subject is a human.

The treatments and diagnostics of any preceding aspect may be administered in such amounts, time, and route deemed necessary in order to achieve the desired result. The exact amount of the treatments and diagnostics of any preceding aspect will vary from subject to subject, depending on the species, age, and general condition of the subject, its mode of administration, its mode of activity, and the like. The treatments and diagnostics of any preceding aspect are preferably formulated in dosage unit form for ease of administration and uniformity of dosage. It will be understood, however, that the treatments and diagnostics of any preceding aspect will be decided by the attending physician within the scope of sound medical judgment. The specific therapeutically effective dose level for any particular subject will depend upon a variety of factors including the disease or pseudodisease being treated and its severity; the activity of the treatments and diagnostics of any preceding aspect employed; the specific treatments and diagnostics of any preceding aspect employed; the age, body weight, general health, sex and diet of the patient; the time of administration, route of administration, and rate of excretion of the specific treatments and diagnostics of any preceding aspect employed; the duration of the treatment; drugs used in combination or coincidental with the treatments and diagnostics of any preceding aspect employed; and like factors well known in the medical arts.

The treatments and diagnostics of any preceding aspect may be administered by any route. In some embodiments, the composition is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the composition (e.g., its stability in the environment of the gastrointestinal tract), the condition of the subject (e.g., whether the subject is able to tolerate oral administration), etc.

The exact amount of the treatments and diagnostics of any preceding aspect required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Machine Learning and Artificial Intelligence Workflows for Brain Cancer Management

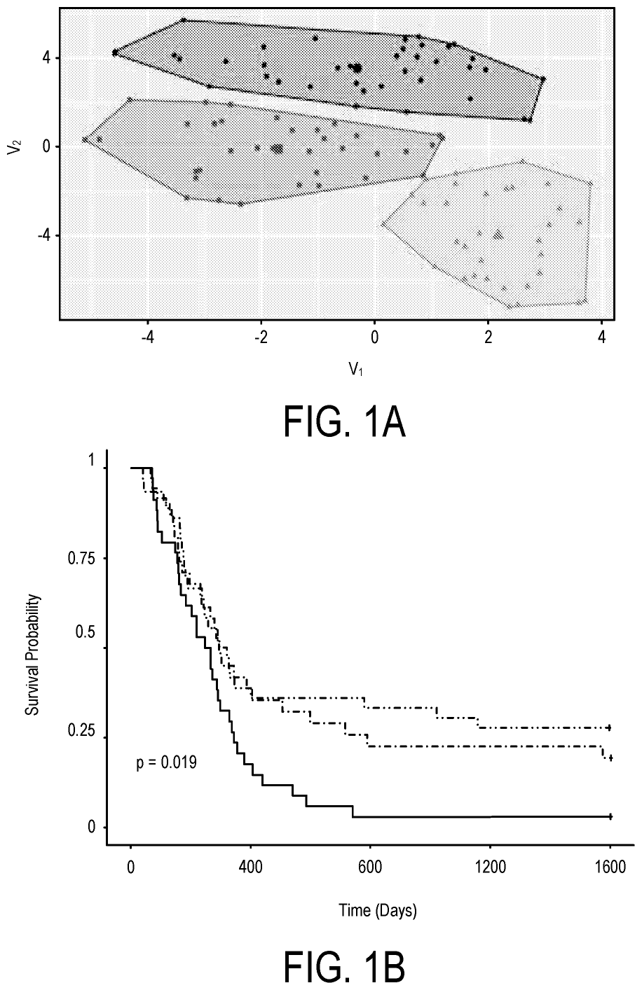

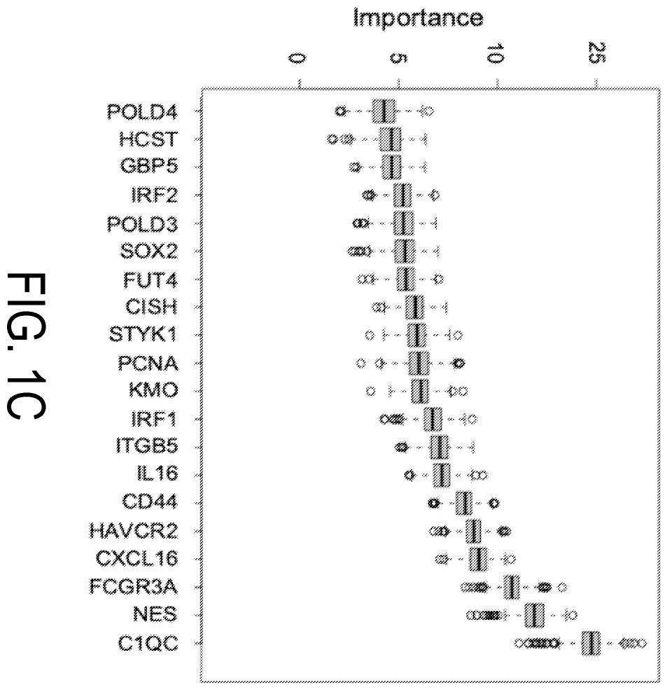

Disclosed herein is an exploration of the underlying transcriptomic responses in the two post-surgical responses of cancer progression (PD) and pseudo-progression (psPD) to (i) explore the underlying molecular drivers of psPD response and (ii) identify novel biomarkers that better stratify PD and psPD. Additionally, disclosed is integration with histologic imaging to identify histological markers which act as surrogate markers for our identified biomarkers. Based upon preliminary data using online recurrent GBM RNASeq data, novel clusters were identified utilizing a tSNE dimensionality reduction approach which evidenced differential survival of cancer patients. Furthermore, exploring the specific expression profiles which stratified these groups using a Random Forest-based learning model, leading factors included genes related to immune response and stem cell-like activity (FIG. 1).

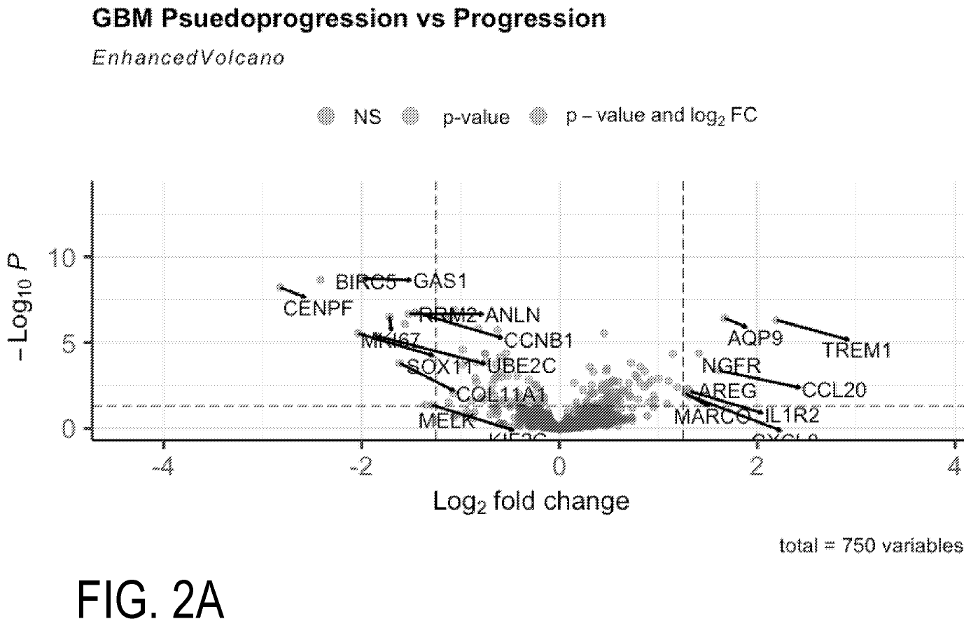

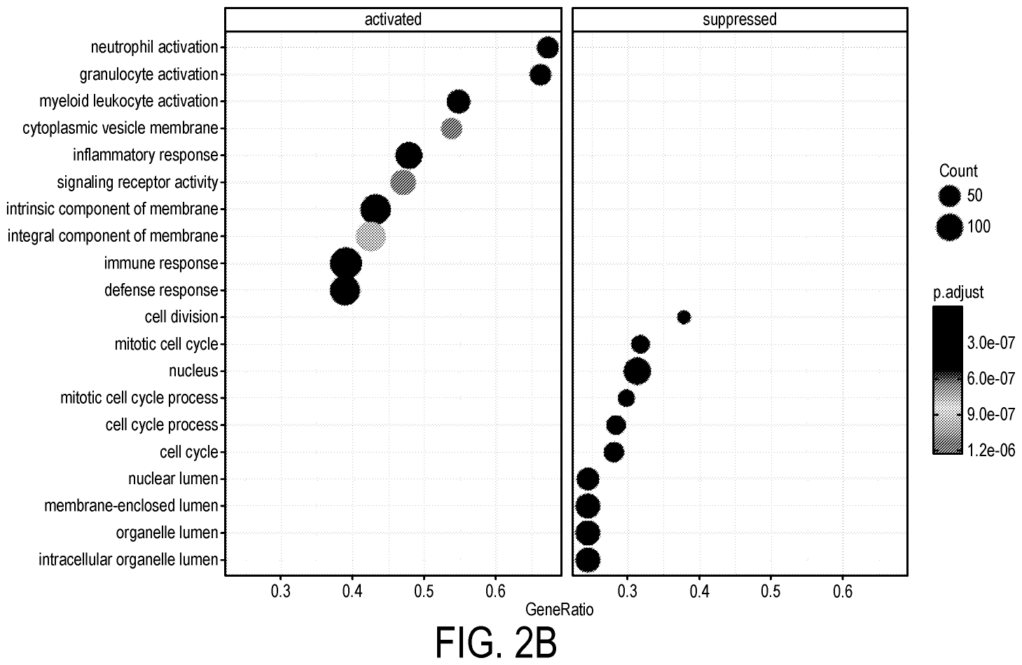

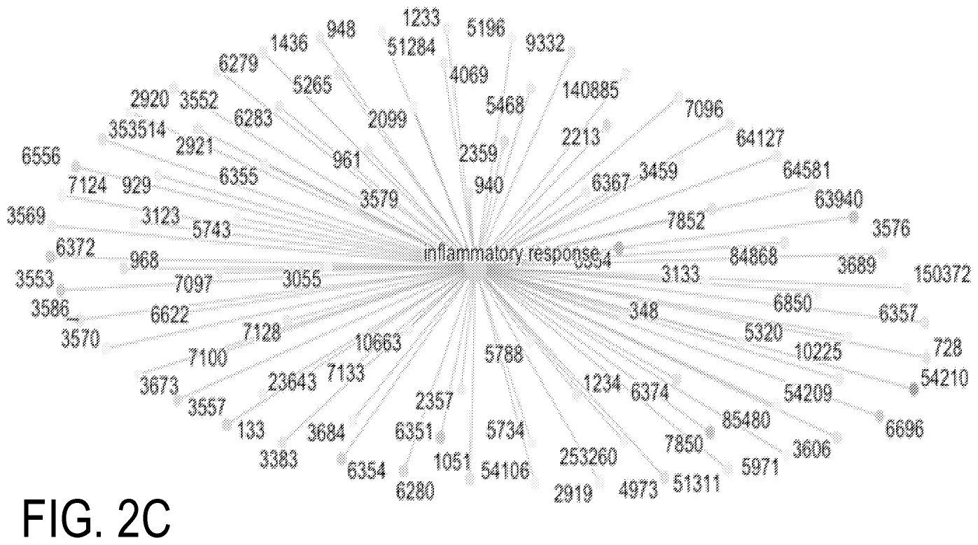

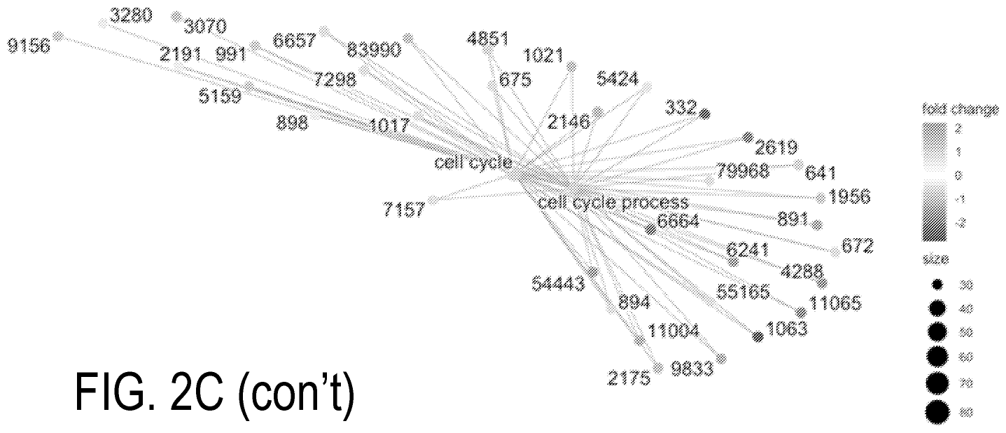

To further explore these responses, approximately 60 lesion samples taken from GBM patients were obtained from tissue archive services and confirmed as either PD or psPD lesions. Samples were extracted for RNA and validated for RNA quality using Nanodrop, Qubit and Tapestation. After quality control analysis, 48 samples were selected for Nanostring. Differential expression analysis identified prominent downregulated genes related to stem-cell like activity and proliferation (CENPF, MK167, SOX11, etc) and several upregulated genes related to immune cell recruitment and activation (TREM1, CCL20, AQP9, etc) in psPD samples when compared with PD samples (FIG. 2A). These results thus indicated that psPD lesions can be driven by prominent inflammatory responses, while PD lesions have active stem-cell like proliferation. To further explore this, gene enrichment analysis and network analysis was assessed to highlight prominent down regulation of cell proliferating response and upregulation of immune response in psPD lesions compared to PD lesions (FIG. 2B-2C).

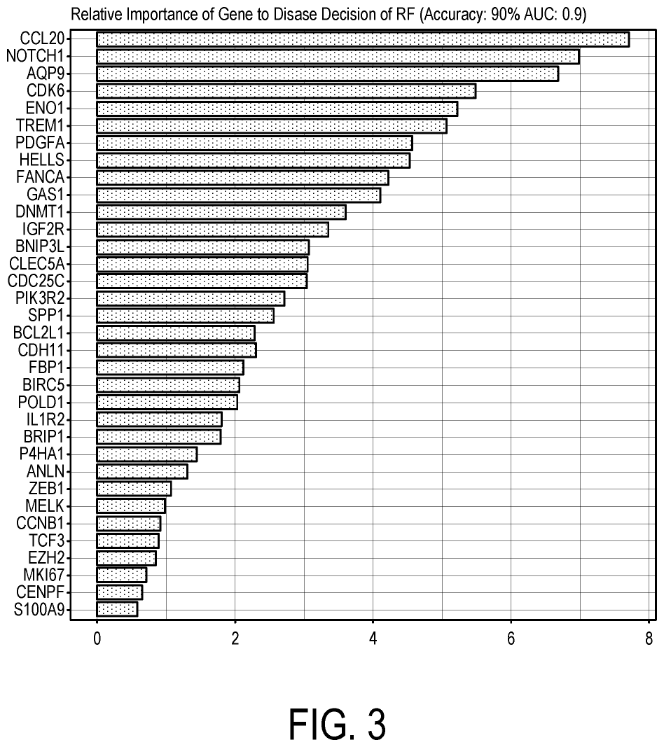

To further assess the predictive capacity of such genes as diagnostic biomarkers, a random forest model was trained utilizing 60% of the samples and tested with remaining 40% of samples to predict PD or psPD. This model was able to accurately predict disease with a confusion matrix accuracy of 90% and area under the curve (AUC) score of 0.9. Moreover, assaying the relevant genes that best drove model decisions, several genes related to immune cell recruitment, response and cellular proliferation were utilized (FIG. 3).

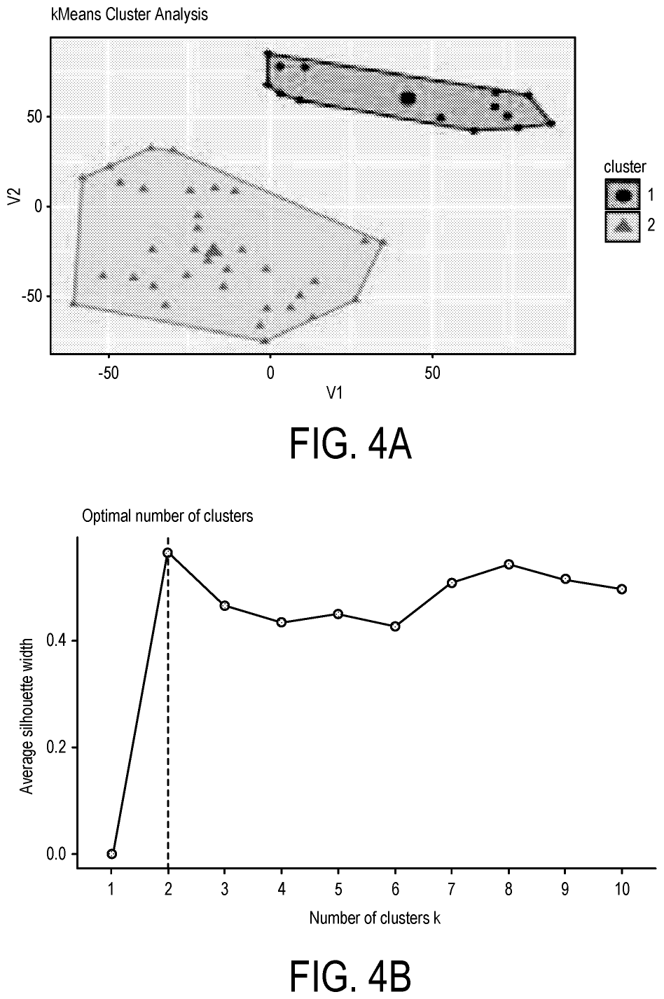

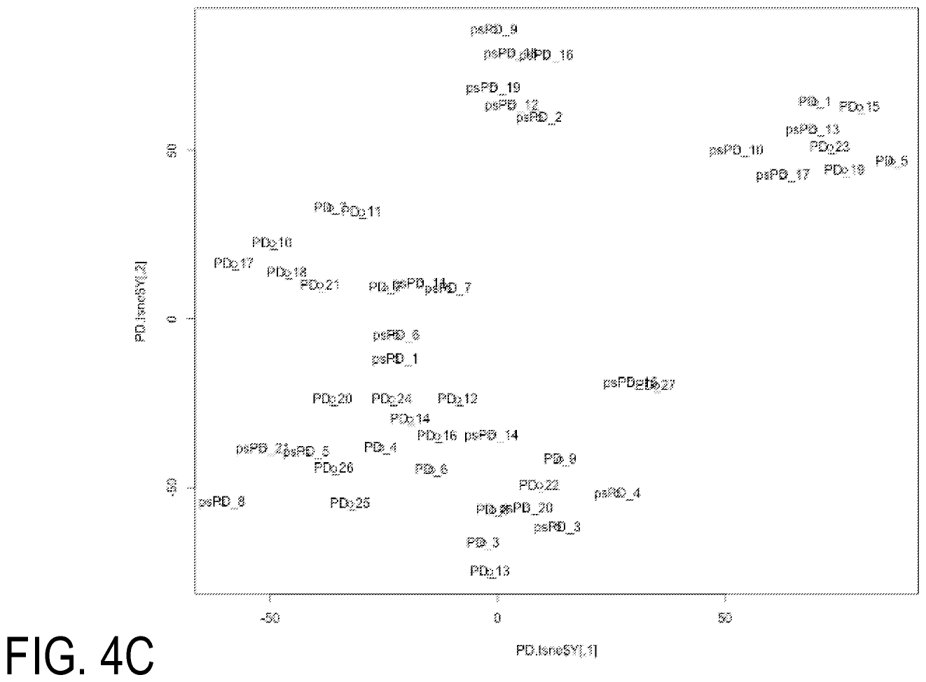

However, to assess whether specific samples grouped differently based on expression profiles, tSNE dimensionality reduction was performed using expression profiles independent of ground truth labeling. Subsequent kMeans analysis identified 2 statistically distinct clusters with mixed presentation of PD or psPD lesions within a single cluster (FIG. 4). Suggesting the possibility that existing histologic diagnostics inaccurately categorize disease based on transcriptional findings.

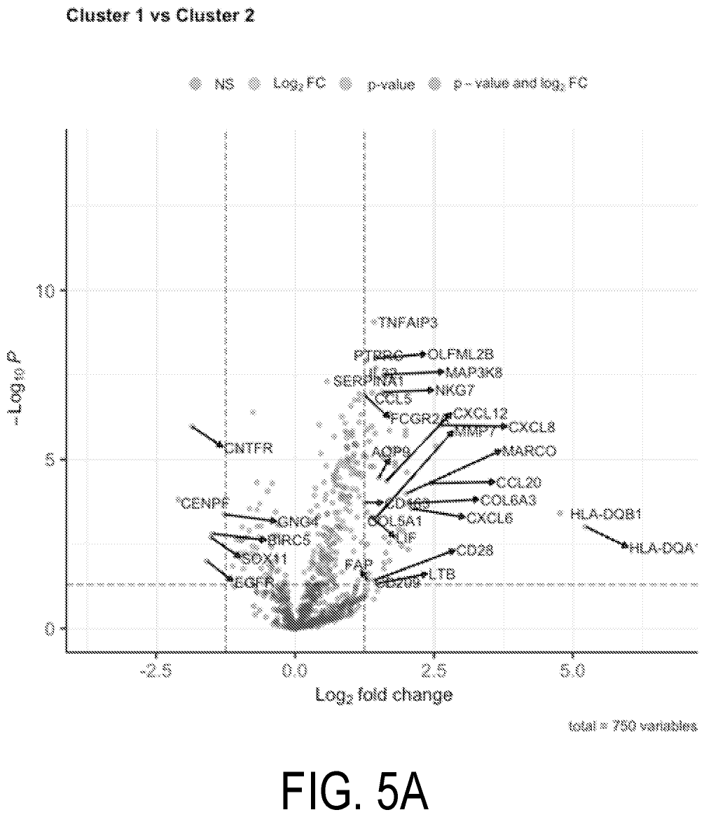

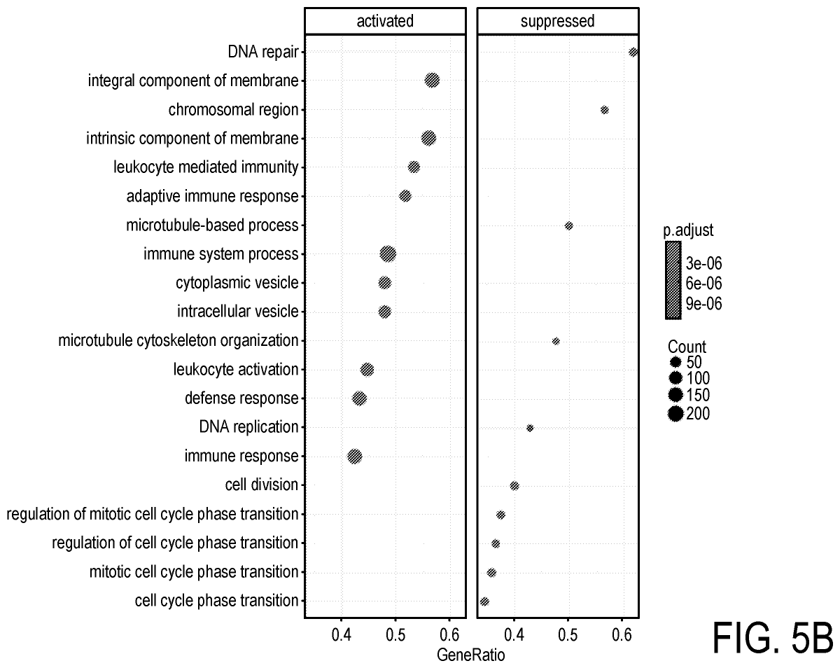

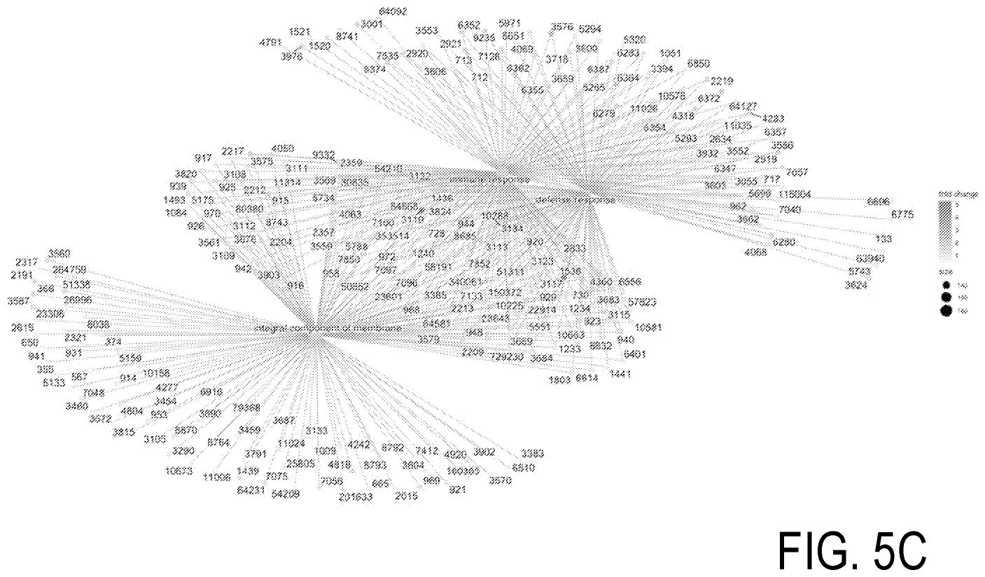

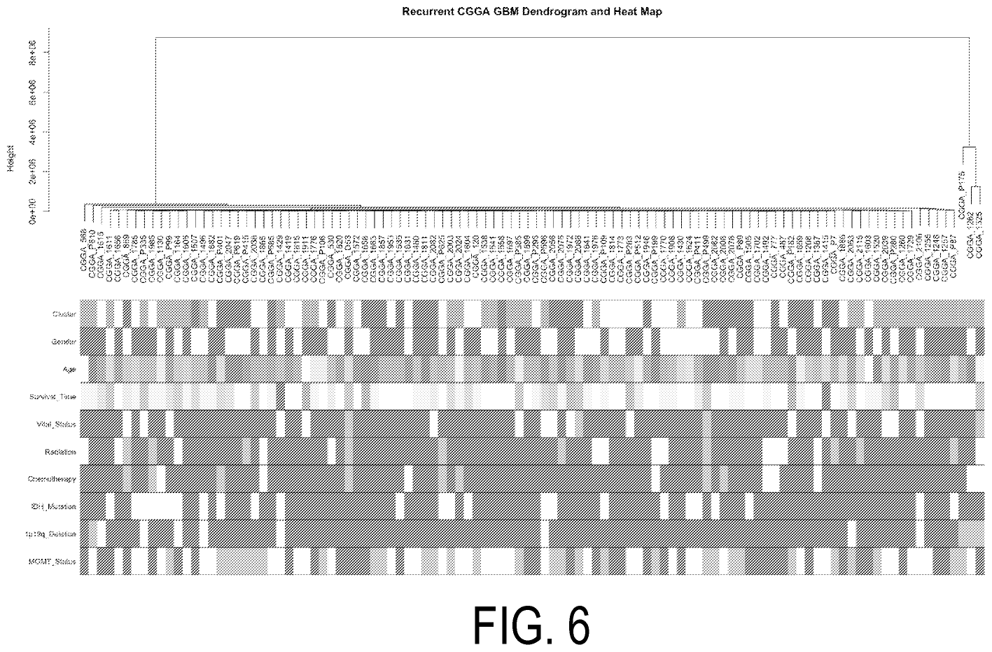

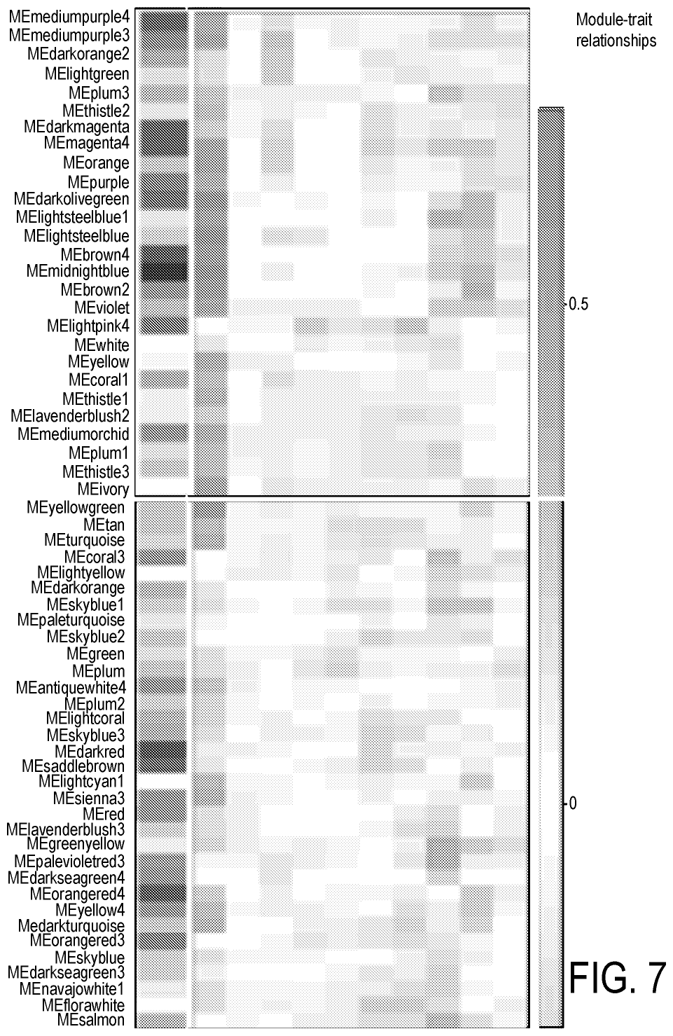

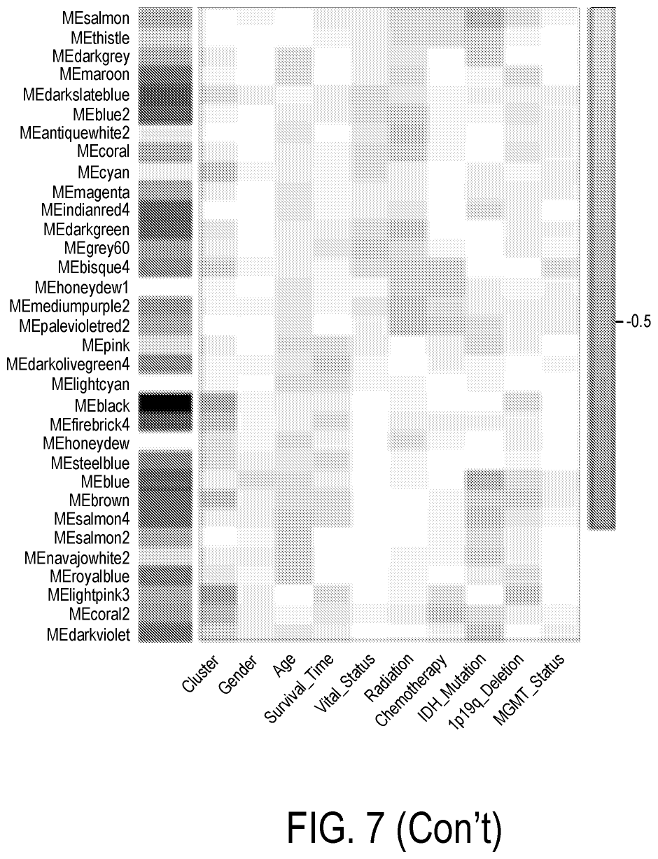

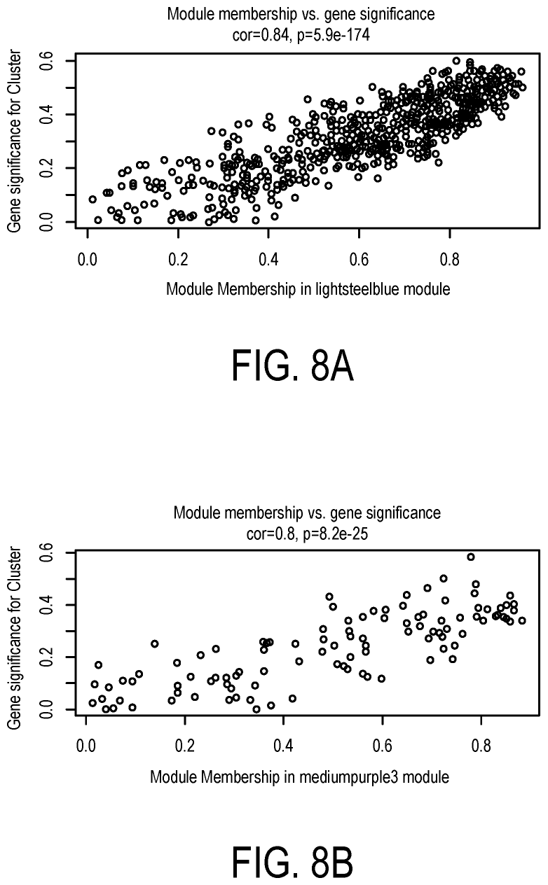

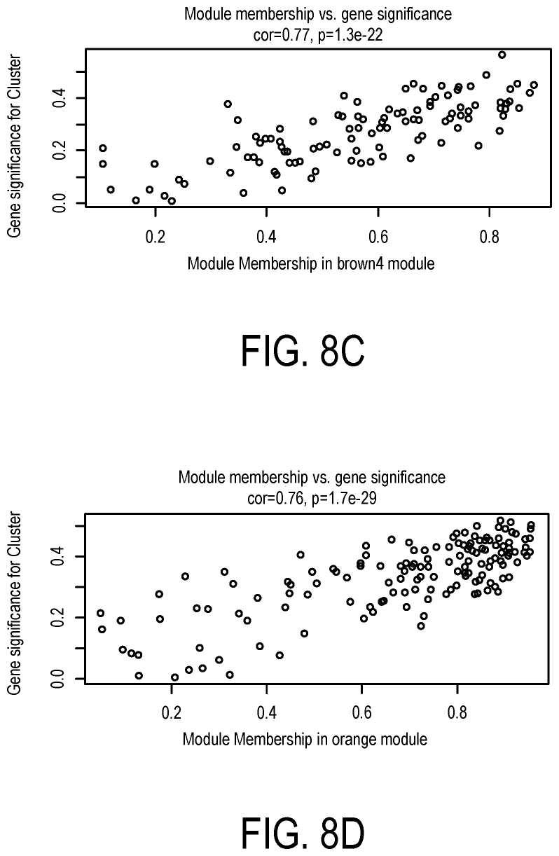

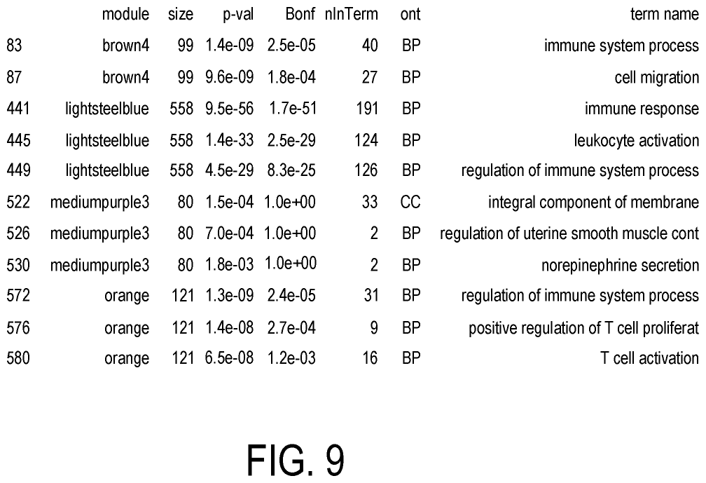

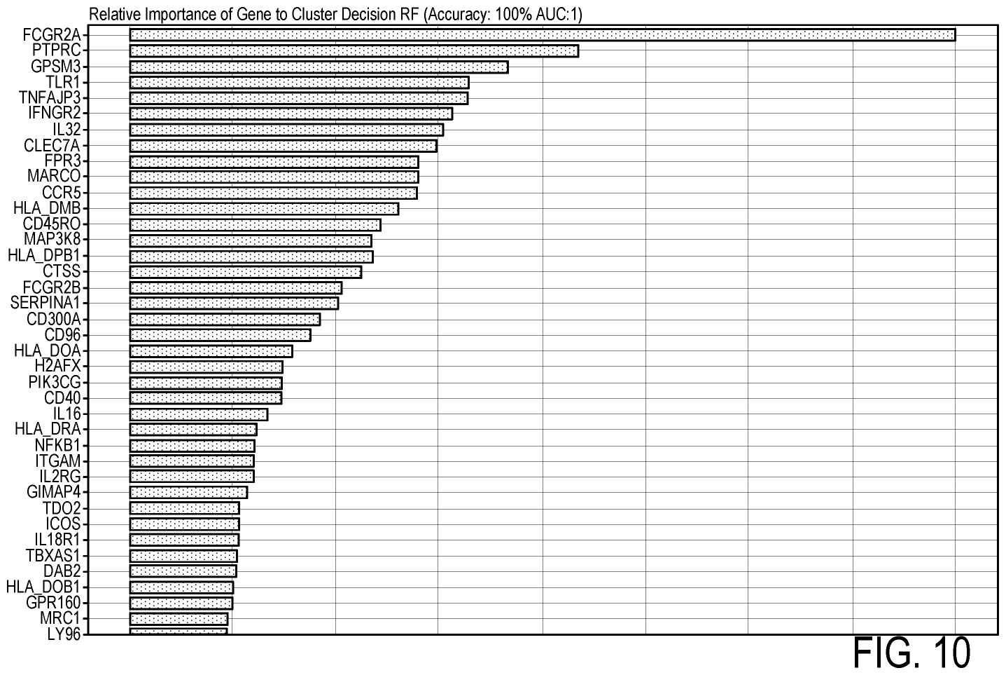

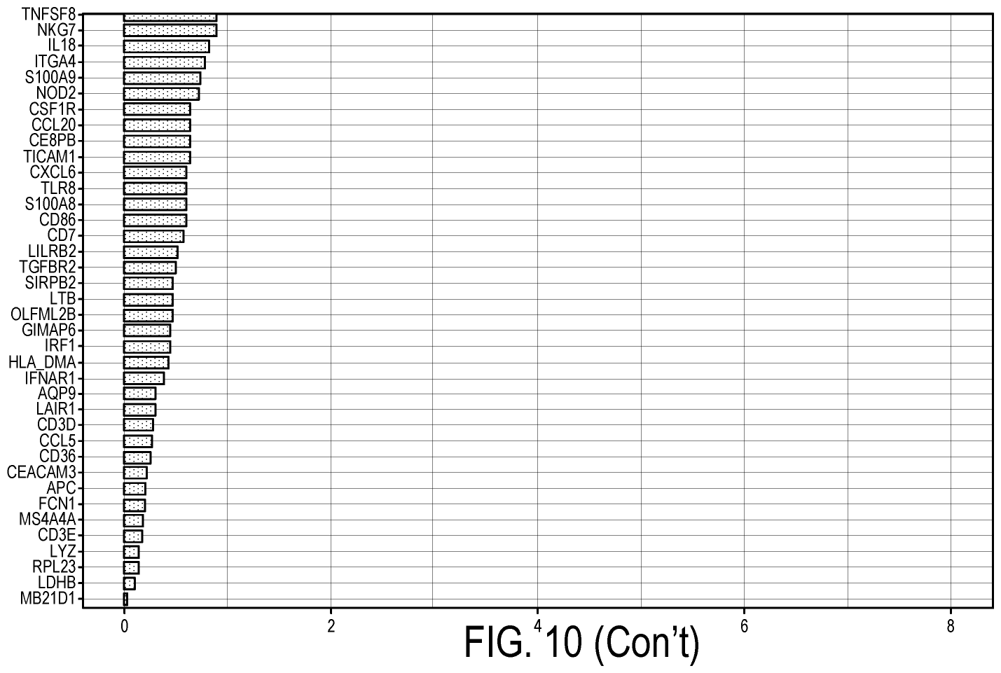

Differential expression, pathway, and network analyses were performed comparing cluster 1 to cluster 2 (FIG. 5). Notably, immune cell response pathways were increased in expression in cluster 1 compared to cluster 2, highlighting a use of immune cell response to differentiate the two pathways. Furthermore, when assaying the capacity of a random forest algorithm in predicting a cluster, confusion matrix accuracy scores were 100% with an AUC of a (FIG. 10). Specifically, the decision modeling was heavily driven by expression of genes related to immune response.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

1. A method for identifying a subject with progressing glioblastoma versus pseudo-progressing glioblastoma, the method comprising:
a. determining an expression status of one or more genes selected from 1000 genes or more isolated from a biological sample from the subject;
b. using the expression status to determine an expression score relative to a normal subject;
c. identifying a subject with an expression score greater than 25% as being at risk of having progressing glioblastoma relative to a normal subject;
d. identifying a subject with an expression score less than 25% as being at risk of having pseudo-progressing glioblastoma relative to a normal subject; and
e. treating the subject based on the expression score.

2. The method of claim 1, wherein the expression score is determined from the expression status of a subset of genes

3. The method of claim 1, wherein the expression score is determined from the expression status of a subset of at least 10 genes.

4. (canceled)

5. (canceled)

6. (canceled)

7. The method of claim 1, wherein the subject identified with progressing glioblastoma is treated with surgery, chemotherapy, radiation, immunotherapy, gene therapy, or a combination thereof.

8. The method of claim 1, wherein the subject identified with pseudo-progressing glioblastoma is treated with monitoring and/or steroids.

9. The method of claim 1, wherein the biological sample is selected from the group consisting of blood, plasma, serum, cerebrospinal fluid, urine, saliva, stool, synovial fluid, a solid tissue sample, and a cell culture sample.

10. A method for identifying a subject with progressing glioblastoma and further diagnosing them to confirm that the subject has progressing glioblastoma, the method comprising:
a. determining an expression status of one or more genes selected from 1000 genes or more isolated from a biological sample from the subject;
b. using the expression status to determine an expression score relative to a normal subject;
c. identifying a subject with an expression score greater than 25% as being at risk of having progressing glioblastoma relative to a normal subject; and
d. subjecting the subject to a further diagnosis.

11. The method of claim 10, wherein the expression score is determined from the expression status of a subset of genes

12. The method of claim 10, wherein the expression score is determined from the expression status of a subset of at least 10 genes.

13. (canceled)

14. (canceled)

15. (canceled)

16. The method of claim 10, wherein said further diagnosis comprises an MRI.

17. The method of claim 10, wherein after the further diagnosis, the subject is treated with surgery, chemotherapy, radiation, immunotherapy, gene therapy, or a combination thereof.

18. The method of claim 10, wherein the biological sample is selected from the group consisting of blood, plasma, serum, cerebrospinal fluid, urine, saliva, stool, synovial fluid, a solid tissue sample, and a cell culture sample.

19. A method for monitoring progression in a subject undergoing a treatment for a glioblastoma and adjusting said treatment, the method comprising:
a. determining an expression status of one or more genes selected from 1000 genes or more isolated from a biological sample from the subject;
b. using the expression status to determine an expression score relative to a normal subject;
c. identifying a subject with an expression score greater than 25% as being at risk of having progressing glioblastoma relative to a normal subject;
d. giving the subject a first treatment based on the expression score;
e. repeating steps, a)-d) to monitor a change in the expression score; and
f. giving the subject a second treatment if the expression score remains or increases above 25%.

20. The method of claim 19, wherein the expression score is determined from the expression status of a subset of genes

21. The method of claim 19, wherein the expression score is determined from the expression status of a subset of at least 10 genes.

22. (canceled)

23. (canceled)

24. (canceled)

25. The method of claim 19, wherein the second treatment is given alone or in combination with the first treatment.

26. (canceled)

27. The method of claim 19, wherein the first treatment and the second treatment is surgery, chemotherapy, radiation, immunotherapy, gene therapy, or a combination thereof.

28. The method of claim 19, wherein the biological sample is selected from the group consisting of blood, plasma, serum, cerebrospinal fluid, urine, saliva, stool, synovial fluid, a solid tissue sample, and a cell culture sample.

29. The method of claim 1, wherein the subject is a human.

30. A diagnostic kit or assay for identifying a subject with progressing glioblastoma, wherein the kit or assay comprises components comprising one or more detection means for detecting one or more biomarkers selected from the group comprising CENPF, BIRC5, GAS1, ANLN, RRM2, MKI67, SOX11, CCNB1, UBE2C, CQL11A, MELK, AQP9, NGFR, TREM1, AREG, MARCO, IL1R2, CCL20, NOTCH1, CDK6, ENO1, TREM, PDGFA, HELLS, FANCA, DNMT1, IGF2R, BNIP3L, CLEC5A, CDC25C, PIK3R2, SPP1, BCL2L1, CDH11, FBP1, BIRC5, POLD1, IL1R2, BRIP1, P4HA1, ZEB1, TCF3, EZH2, S100A9, or combinations thereof.

31-42. (canceled)
